(19) United States
(12) Patent Application Publication
(10) Pub. No.: US 2024/0219393 A1
(43) Pub. Date: Jul. 4, 2024

(54) ALUMINOSILICATE NANOPARTICLE SENSORS AND USES THEREOF

(71) Applicant: CORNELL UNIVERSITY, Ithaca, NY (US)

(72) Inventors: Rachel Lee, Ithaca, NY (US); Jacob Erstling, Ithaca, NY (US); Joshua Hinckley, Framingham, MA (US); Ulrich B. Wiesner, Ithaca, NY (US)

(21) Appl. No.: 18/555,647

(22) PCT Filed: Apr. 15, 2022
(86) PCT No.: PCT/US22/25072

§ 371 Date: Oct. 16, 2023

Related U.S. Application Data
(60) Provisional application No. 63/175,392, filed on Apr. 15, 2021.

(51) Int. Cl. G01N 33/58; C09K 11/02; C09K 11/06; G01N 15/01; G01N 15/075

(52) U.S. Cl. CPC G01N 33/587; C09K 11/025; C09K 11/06; G01N 15/01; G01N 15/075; G01N 33/582

(57) ABSTRACT

Methods of determining the presence or absence or local concentration of an analyte in a sample or an individual or a portion thereof using ratiometric sensing and optical super-resolution microscopy (OSRM). The methods use aluminosilicate nanoparticles that can be used in OSRM. The analytes can be biologically relevant analytes, such as, for example, biologically relevant hydrogen ions, oxygen, reactive oxygen species, anions, nitric oxide, metal ions, anions, etc. The methods utilize averaging to address aluminosilicate nanoparticle homogeneity. The methods can be used in methods of treatment.

### CROSS-REFERENCE TO RELATED APPLICATIONS

This application claims the benefit of U.S. Provisional Patent Application No. 63/175,392, filed Apr. 15, 2021, the contents of the above-identified application are hereby fully incorporated herein by reference in their entirety.

### STATEMENT REGARDING FEDERALLY SPONSORED RESEARCH

This invention was made with government support under grant numbers CA199081 and RR025502 awarded by the National Institutes of Health. The government has certain rights in the invention.

### BACKGROUND OF THE DISCLOSURE

Developing nanoscale optical probes to quantitatively interrogate local biological environments is an important area of nanoscale science and engineering. Furthermore, understanding cellular trafficking and processing of nanoparticles is important to elucidating the fundamental mechanisms of how nanoparticles execute their designed function in a living organism. Certain classes of nanoparticles are known to be taken up by cells in membrane-enclosed compartments, and these compartments have been reported to have distinct chemical microenvironments as they go through the stages of cellular processing. One of the most studied chemical indicators of these cellular compartments is their pH, and a direct readout of the pH experienced by a nanoparticle can be used to provide information about its local environment. Developing pH sensing probes ideally requires independence from probe concentration and nonuniform excitation so that there are no limits to the target area detected. Ratiometric sensors have been constructed for this purpose, which typically comprise two mutually non-interfering sources of signals: A reference signal independent of the sensing target, and a sensor signal strongly dependent on changes in the sensor target chemical concentration. Among the most widely used techniques for investigating cellular processes, fluorescence microscopy provides spatial information, can be performed in real-time and on often readily available instrumentation, making it user friendly and generalizable. However, challenges in ratiometric fluorescent sensing for tracking microenvironments remain, including spatial resolution, signal-to-noise ratio, general stability and biocompatibility of the sensing probe, as well as accounting for inaccuracies in sensing results as a function of compositional heterogeneities of the ratiometric sensor probes, in particular for solution-synthesized particle-based multi-dye systems.

Relative to other microscopies including electron and atomic force microscopies, spatial resolution has been an obstacle in conventional optical fluorescence imaging, including ratiometric sensing. Processes and structures below the diffraction limit cannot directly be distinguished, rendering understanding of underlying molecular processes difficult. Optical microscopy techniques that increase the resolving power beyond the diffraction limit are referred to as “super-resolution microscopy” (SRM). While other nanoparticle-based techniques exist, allowing for spatial-resolution-enhancement in cell imaging, such as expansion microscopy (ExM), super-resolution optical fluctuation imaging (SOFI), and photoacoustic imaging (PA), two of the most prominent techniques are stimulated emission depletion (STED) microscopy and the single molecule localization microscopy (SMLM) referred to as stochastic optical reconstruction microscopy (STORM). Super-resolution pH sensing has been achieved before in live-cell STED microscopy; however, STED requires a specialized optical setup not readily available except to experts, thereby limiting access. STORM microscopy, on the other hand, can be performed on standard fluorescence microscopy instrumentation, but has special requirements for imaging probes that can be photoswitched reversibly between “on” and “off” states. Reversible and stochastic photoswitching, or blinking, allows for identification and localization of individual emitters on separate camera/detector frames, leading to super-resolution images upon summing up of all individually processed frames taken over a given time period. Despite STORM's relatively straight forward and common instrumentation requirements, typical current photoswitching-inducing setups involve cytotoxic imaging conditions that disable live-cell imaging. Representative components of such setups consist of an excitation laser for the fluorophore, a UV activation laser, a thiol source (commonly beta-mercaptoethanol), and an oxygen scavenging system. The UV activation laser may cause cell damage, the thiol compound is often cytotoxic, and the oxygen scavenging system may alter the pH level in the imaging solution, the first two of which, in particular, render these types of setups incompatible with live-cell imaging.

### SUMMARY OF THE DISCLOSURE

In an aspect, the present disclosure provides methods of determining a presence or an absence of an analyte or a concentration of an analyte in a sample or a portion thereof or an individual or a portion thereof. In various examples, a method of determining a presence or an absence of an analyte or a concentration of an analyte in a sample or a portion thereof or an individual or a portion thereof using one or more aluminosilicate nanoparticle(s) comprises: contacting the sample or individual with a plurality of aluminosilicate nanoparticles, each aluminosilicate nanoparticle comprising: one or more reference dye groups(s), where each reference dye group is covalently bound to and encapsulated in the network of the aluminosilicate nanoparticle, one or more sensing dye groups(s), where the sensing groups are capable of interacting with the sample or the portion thereof or the individual or the portion thereof, and a plurality of polyethylene glycol (PEG) groups disposed on at least a portion of an surface or all of the surfaces of the aluminosilicate nanoparticle; optionally, incubating the aluminosilicate nanoparticles with the sample or in the individual; determining a presence or an absence or a concentration of the analyte in an individual region of a first object plane using ratiometric sensing; localizing with resolution below Abbe's diffraction limit at least a portion of or all of the individual aluminosilicate nanoparticles in a second object plane, where the second object plane corresponds to at least a portion or all of the first object plane, using optical super-resolution microscopy (OSRM) imaging; determining a presence or an absence of the analyte or the concentration of the analyte substantially at or at the position of one or more of the aluminosilicate nanoparticles using the presence or the absence or the concentration of the analyte obtained using the ratiometric sensing and the localization of the aluminosilicate nanoparticles obtained using OSRM; and averaging the fluorescence intensity ratio of a desired number of individual aluminosilicate nanoparticles in proximity to an individual aluminosilicate nanoparticle to assign an average fluorescence intensity ratio to the individual aluminosilicate nanoparticle, where the average fluorescence intensity ratio assigned to the individual aluminosilicate nanoparticle corresponds to the presence or the absence of the analyte or the concentration of the analyte in the sample or the portion thereof or the individual or the portion thereof. In various examples, the determining the presence or the absence or the local concentration of the analyte in the individual region of a detecting plane using ratiometric sensing and the localizing with resolution below Abbe's diffraction limit at least a portion of or all of the individual aluminosilicate nanoparticles in the second object plane are each carried out using OSRM imaging. In various examples, the presence or the absence of the analyte or the concentration of the analyte in the sample or the portion thereof or the individual or the portion thereof is determined substantially at one or more of the aluminosilicate nanoparticle(s). In various examples, the method comprises an OSRM method chosen from ground state depletion (GSD) microscopy, stochastic optical reconstruction microscopy (STORM), direct stochastic optical reconstruction microscopy (dSTORM), stimulated emission and depletion (STED), and photoactivated localization microscopy (PALM). In various examples, the contacting is administering the composition to the individual.

In various examples, the aluminosilicate nanoparticles are chosen from: aluminosilicate core-organic ligand shell nanoparticles, each of the aluminosilicate core-organic ligand shell nanoparticles comprising: an aluminosilicate core, one or more reference dye group(s) covalently bound to and encapsulated in the aluminosilicate network of the aluminosilicate core-organic ligand shell nanoparticle, one or more sensing dye group(s) capable of analyte sensing covalently bound to the aluminosilicate core network, where the one or more reference dye group(s) and the one or more sensing dye group(s) do not interfere with each other and/or one or more sensing dye group(s) capable of analyte sensing is/are disposed on at least a portion of or all of a surface or at least a portion of or all of the surfaces of the aluminosilicate core, and a plurality of PEG groups disposed on at least a portion of a surface or all of the surfaces of the aluminosilicate core; aluminosilicate core-aluminosilicate shell-organic shell nanoparticles, each of the aluminosilicate core-aluminosilicate shell-organic shell nanoparticles comprising: an aluminosilicate core, one or more reference dye group(s) covalently bound to and encapsulated in the aluminosilicate network of the aluminosilicate core, an aluminosilicate shell disposed on at least a portion of or all of a surface or at least a portion of or all of the surfaces of the aluminosilicate core, one or more sensing dye group(s) capable of analyte sensing covalently bound to and encapsulated in the aluminosilicate network of the aluminosilicate shell, optionally, one or more sensing dye group(s) capable of analyte sensing disposed on at least a portion of or all of a surface or a portion of or all of the surfaces of the aluminosilicate shell, and a plurality of PEG groups disposed on at least a portion of a surface or all of the surfaces of the aluminosilicate shell; and any combination thereof. In various examples, the aluminosilicate nanoparticles individually have at least one dimension of about 2 nm to about 10 nm. In various examples, the aluminosilicate nanoparticles individually further comprise one or more targeting group(s), one or more therapeutic group(s), one or more diagnostic group(s), or any combination thereof.

In various examples, the analyte is chosen from hydrogen ions, oxidants, antioxidants, oxygen, reactive oxygen species (ROS), nitric oxide, chloride ions, metals, and metal ions. In various examples, the analyte is hydrogen ions and the local pH substantially at or at the position of at least a portion or all of the aluminosilicate nanoparticles in the sample or the portion thereof or the individual or the portion thereof is determined. In various examples, the individual sensing dye group(s) is/are capable of sensing pH, sensing redox status, sensing the presence or absence of oxygen, sensing the presence or absence of reactive oxygen species (ROS), sensing the presence or absence of chloride ions, sensing the presence or absence of nitric oxide, or sensing the presence or absence of one or more metal(s) and/or metal ion(s).

In various examples, a method of targeting, diagnosing, treating, preventing, or any combination thereof, a current or potential disease, disease state, condition, disorder, side effect, or any combination thereof, in an individual comprises a method of determining a presence or an absence of an analyte or a concentration of an analyte in a sample or a portion thereof or an individual or a portion thereof of the present disclosure. In various examples, the sample is a biopsy sample or a resected tissue sample. In various examples, the current or potential disease, disease state, condition, disorder, side effect, or any combination thereof, is chosen from infections, cancers, neurological conditions/diseases, neurodegenerative diseases, psychological conditions/diseases, inflammatory conditions/diseases, cardio-vascular diseases, and any combination thereof. In various examples, the current or potential disease is cancer, and the method further comprises one or more chemotherapy treatment(s), one or more radiation treatment(s), one or more photodynamic therapy treatment(s), one or more surgical intervention(s), or the like, or any combination thereof. In various examples, the method further comprises visualization of abnormal cells after administration of the aluminosilicate nanoparticles. In various examples, the visualization is carried out using fluorescence imaging.

In various examples, a kit comprises one or more (e.g., a plurality of) aluminosilicate nanoparticles and/or a composition comprising the aluminosilicate nanoparticle(s), and instructions for use of the aluminosilicate nanoparticles and/or the composition(s) for carrying out a method of the present disclosure (e.g., a method of determining a presence or an absence of an analyte or a concentration of an analyte in a sample or a portion thereof or an individual or a portion thereof of the present disclosure or a method of targeting, diagnosing, treating, preventing, or any combination thereof, a current or potential disease, disease state, condition, disorder, side effect, or any combination thereof, in an individual). In various examples, the aluminosilicate nanoparticles are chosen from: aluminosilicate core-organic ligand shell nanoparticles, each of the aluminosilicate core—organic ligand shell nanoparticles comprising: an aluminosilicate core, one or more reference dye group(s) covalently bound to and encapsulated in the aluminosilicate network of the aluminosilicate core-organic ligand shell nanoparticle, one or more sensing dye group(s) capable of analyte sensing covalently bound to the aluminosilicate core network, where the one or more reference dye group(s) and the one or more sensing dye group(s) do not interfere with each other and/or one or more sensing dye group(s) capable of analyte sensing is/are disposed on at least a portion of or all of a surface or at least a portion of or all of the surfaces of the aluminosilicate core, and a plurality of PEG groups disposed on at least a portion of a surface or all of the surfaces of the aluminosilicate core; aluminosilicate core-aluminosilicate shell-organic shell nanoparticles, each of the aluminosilicate core-aluminosilicate shell-organic shell nanoparticles comprising: an aluminosilicate core, one or more reference dye group(s) covalently bound to and encapsulated in the aluminosilicate network of the aluminosilicate core, an aluminosilicate shell disposed on at least a portion of or all of a surface or at least a portion of or all of the surfaces of the aluminosilicate core, one or more sensing dye group(s) capable of analyte sensing covalently bound to and encapsulated in the aluminosilicate network of the aluminosilicate shell, optionally, one or more sensing dye group(s) capable of analyte sensing disposed on at least a portion of or all of a surface or a portion of or all of the surfaces of the aluminosilicate shell, and a plurality of PEG groups disposed on at least a portion of a surface or all of the surfaces of the aluminosilicate shell; and any combination thereof. In various examples, the aluminosilicate nanoparticles individually have at least one dimension of about 2 to about 10 nm.

### BRIEF DESCRIPTION OF THE FIGURES

For a fuller understanding of the nature and objects of the disclosure, reference should be made to the following detailed description taken in conjunction with the accompanying figures located in the Examples.

FIGS. 1A-1C show: (FIG. 1A) molecular rendering and (FIG. 1B) illustration of nanosensor particle architecture and (FIG. 1C) molecular structure of reagents used. The rendering only shows an aluminosilicate core (representative aluminum (Al) atoms indicated) with covalently encapsulated ATTO647N-maleimide (ATTO647N) reference dye (a red emitting fluorescent dye) and 6-Fluorescein-azide (FAM) sensor dye (a green emitting fluorescent dye) covalently attached to the core surface (FAM-ATTO647N aC′ dot) (for simplicity, a polyethylene glycol (PEG) particle shell is not shown).

FIGS. 2A-2M show gel-permeation chromatography (GPC) elution profiles of FAM-ATTO647N (FIG. 2A) aC′ dot and (FIG. 2B) C′ dot nanosensors; fluorescence correlation spectroscopy (FCS) correlation curves with fits of FAM-ATTO647N (FIG. 2C) aC′ dots (hydrodynamic size=4.8 nm) and (FIG. 2D) C′ dots (hydrodynamic size=6.7 nm); ultraviolet/visible (UV/Vis) spectra of FAM-ATTO647N (FIG. 2E) aC′ dots and (FIG. 2F) C′ dots; changes of fluorescence as a function of pH for FAM sensor (left) and ATTO647N reference (right) dyes in FAM-ATTO647N (FIG. 2G) aC′ dot and (FIG. 2H) C′ dot solutions (both FAM sensor and ATTO647N reference dye traces display superimposed individual spectra recorded in all pH conditions tested); ratiometric calibration curves derived therefrom for FAM-ATTO647N (FIG. 2I) aC′ dots and (FIG. 2J) C′ dots; comparison of solution-based pH calibration curves (obtained from fluorometer work; see FIG. 5), displaying FAMIATTO647N intensity ratios as a function of known solution pH, with FAMIATTO647N intensity ratios derived from single-particle based experiments showing results (FIG. 2K) for individual isolated particles, (FIG. 2L) for averages over regions containing 10 particles, or (FIG. 2M) averages over regions containing 25 particles.

FIGS. 3A-3B show transmission electron microscopy (TEM) images of (FIG. 3A) FAM-ATTO647N aC′ dots and (FIG. 3B) FAM-ATTO647N C′ dots.

FIG. 4 shows relative brightness per dye of aC′ dots compared to free ATTO647N and FAM in PBS solutions at various pH values.

FIGS. 5A-5F show: Calibration curves (FIGS. 5A, 5C, 5E) and Henderson-Hasselbalch analysis (FIGS. 5B, 5D, 5F), including separate data sets obtained from solution (fluorimeter) and microscopy work, of (FIGS. 5A-5B) FAM-ATTO647N C′ dots (additional data sets from confocal microscopy), (FIGS. 5C-5D) FAM-ATTO647N aC′ dot (additional data sets from confocal microscopy), and (FIGS. 5E-5F) FAM-ATTO647N aC′ dot (additional data sets from TTRF microscopy). Biologically relevant pH ranges were chosen for the final linear fits in (FIGS. 5B, 5D, 5F).

FIG. 6 shows two representative sets of cropped fluorescence image regions displaying FAM (sensor) and ATTO647N (reference) dye channel signals of FAM-ATTO647N aC′ dots immobilized on a glass surface under TIRF microscopy. White arrows (W) indicate local areas within these regions where FAM signal exists without corresponding ATTO647N signal, orange arrows (0) indicate local areas where ATTO647N signal exists without corresponding FAM signal, and yellow arrows (Y) indicate local areas where FAM and ATTO647N signals colocalize. Size bar is 1 micron (μm).

FIG. 7 shows an illustration of compositional/structural particle batch heterogeneities based on different reference (core ovals) and sensor (surface ovals) dye numbers per particle that may occur across particles of a single synthesis batch.

FIG. 8 shows isolated pixels of preselected spots of FAM-ATTO647N aC′dots immobilized on a glass surface under TTRF microscopy where FAM and ATTO647N signals colocalize, chosen from images taken at varying pH. From top to bottom imaging results are shown for three pH conditions as indicated on the upper left side for each pH. For each pH, pixelated FAM signal, ATTO647N signal, and FAMIATTO647N signal ratios are displayed for each isolated cluster of pixels (from an individual spot), interpreted as single particles. Scale bar is 500 nm.

FIGS. 9A-9B show example regions of FAM-ATTO647N aC′dots immobilized on a glass surface under TIRF microscopy with either 10 particles (FIG. 9A) or 25 particles (FIG. 9B) chosen for calculation of particle number based averages of FAMIATTO647N intensity ratios. Number of particles per area was estimated by number of spots visible in the ATTO647N reference dye channel.

FIG. 10 shows representative confocal images of live MDA-MB-231 cells co-labeled with Rab5-RFP and FAM-ATTO647N aC′ dots. Panels show excitation at 633 nm (ATTO647N), 561 nm (Rab5-RFP), overlay of ATTO647N and Rab5-RFP (Merge), and overlay of ATTO647N, Rab5-RFP, Hoechst nucleus stain (excitation at 405 nm), and corresponding brightfield image (Merge+BF). Scale bar is 10 μm.

FIGS. 11A-11C show: (FIG. 11A) Example of fluorescence imaging data set of MDA-MB-231 cells incubated with FAM-ATTO647N C′ dots under a confocal microscope at different incubation time points. Displayed are the FAM sensor dye channel, ATTO647N reference dye channel, resulting pH map from use of the sensor/reference signal ratios together with a calibration (FIGS. 5A-5B), and superposition of pH sensing results with associated bright field (BF) cell images. Cell nuclei are stained with Hoechst 33342. The right side shows results for average cellular pH at different incubation time points from (FIG. 111B) FAM-ATTO647N C′ dots, and (FIG. 11C) FAM-ATTO647N aC′ dots displaying very similar behavior.

FIGS. 12A-12F show total internal reflection fluorescence (TIRF) microscopy images of MDA-MB-231 cells incubated with FAM-ATTO647N aC′ dots for 60 minutes and excitation wavelengths of (FIG. 12A) 488 nm (FAM) and (FIG. 12B) 640 nm (ATTO647N) with 0.5 mW laser power. (FIG. 12C) STORM reconstruction (SR) of an image using 640 nm excitation (ATTO647N) acquired at 10 mW laser power. (FIG. 12D) Binary localization mask (Binary SR) generated from converting pixel height profiles of FIG. 12C into binary intensities (see FIG. 18). (FIG. 12E) Cellular pH map generated by taking the ratios of fluorescence intensities in FIGS. 12A-12B and comparing them to a TIRF microscopy-based calibration curve (see FIGS. 5E-5F). (FIG. 12F) Overlay of FIG. 12E and bright field (BF) image of cell. Scale bars are 10 μm.

FIGS. 13A-13B show three representative time traces (shown in Black, Red, and Blue, respectively) of FAM-ATTO647N aC′ dots exposed to 10 mW of laser power collected with 50 ms integration time at excitation wavelengths of (FIG. 13A) 640 nm (ATTO647N) and (FIG. 13B) 488 nm (FAM). Inset in (FIG. 13B) shows a zoom-in of the first 5 seconds of laser power exposure reflecting the rapid photobleaching that takes place. Resulting duty cycles are shown (DCs)

FIGS. 14A-14D show two frames each of isolated objects found in (FIG. 14A) single particle (C1) TIRF frames of immobilized aC′ dots on a glass surface and (FIG. 14C) live-cell (C2) TIRF frames of aC′ dots internalized into MDA-MB-231 cells, and corresponding intensity line profiles (FIGS. 14B, 14D) taken across their x- and y-axes.

FIGS. 15A-15B show a comparison of absolute (FIG. 15A) and normalized (FIG. 15B) averaged line profiles from live-cell images (upper curve) and immobilized single particle images (lower curve).

FIG. 16 shows localizations within individual clusters associated with aC′ dot sensors in individual vesicles and reconstructed from different collection timeframes (as indicated) throughout the collection time window (50-150 s) together with resulting diffusion analysis based on these localization data sets. Arrows show the uncorrelated directions of motion suggesting that drifts or whole cellular motion is not responsible for these observations.

FIG. 17 shows enlarged panels of FIGS. 12E (left) and 12F (right). (Left) Regions of interest (ROIs) ROI1-1 and ROI1-2 selected from cellular TIRF microscopy-derived pH map are indicated by arrows. (Right) Corresponding overlay with brightfield image.

FIGS. 18A-18L show illustrated representations of (FIG. 18A) pixel intensity profiles from STORM reconstruction, (FIG. 18B) STORM pixel intensity profiles with peak intensities normalized, and (FIG. 18C) corresponding binary STORM intensity profiles after binarizing pixel height profiles. Corresponding STORM image processing examples showing (FIG. 18D) STORM reconstruction-based image, (FIG. 18E) STORM image after maximum intensity normalization, and (FIG. 18F) resulting binary STORM localization mask. Illustration of (FIG. 18G) regular pixelated pH map from intensity ratios of green and red channels in TIRF microscopy. (FIG. 18J) Central pixels of associated STORM-based binary emitter localization mask after intensity normalization and pixel height binarization (see FIGS. 18A-18F and FIG. 5). (FIG. 18H) Overlay of emitter localization central pixels onto pixelated TIRF pH map (without interpolation). (FIG. 18I) Mathematical operation to obtain NN-interpolated emitter pixel values. (FIG. 18K) STORM-enhanced pH map without interpolation resulting from FIG. 18H by multiplication. (FIG. 18L) STORM-enhanced pH map with interpolation resulting from FIG. 18I (displaying only example interpolated pixel Pi, final map will consist of all interpolated pixels resulting from their corresponding NNs where this process is repeated). Illustrations and grid sizes not drawn to scale.

FIGS. 19A-19N show ROI1-1 (FIGS. 19A-19G) from TTRF microscopy image in FIGS. 12E-12F displaying pixelated images in the FAM sensor dye and ATTO647N reference dye channels with laser excitations at (FIG. 19A) 488 nm and (FIG. 19B) 640 nm, respectively, taken with 0.5 mW laser power. (FIG. 19C) Corresponding ratios per pixel (FIGS. 19A-19B) mapped onto a pH scale via calibration (FIGS. 5E-5F). (FIG. 19D) Regular pixelated TIRF image in the ATTO647N reference dye channel taken with 10 mW laser power. (FIG. 19E) Binary localization mask of FIG. 19D after STORM-based emitter localization (in ThunderSTORM), intensity normalization, and pixel height binarization (see FIGS. 18A-18F). (FIG. 19F) STORM-based super-resolution-enhanced pH map without interpolation combining information via multiplication about emitter localizations from (FIG. 19E) with information from pixelated TTRF microcopy-based pH map in FIG. 19C (see FIGS. 18G-18K). (FIG. 19G) STORM-based super-resolution-enhanced NN-interpolated pH map transformed from FIG. 19F (see FIGS. 18G-18L). ROI2-1 (FIGS. 19H-19N) identified in a separate MDA-MB-231 cell (data not shown) incubated with aC′ dot sensor particles for 60 min. Pixelated images in the FAM sensor dye and ATTO647N reference dye channels with laser excitations at (FIG. 19H) 488 nm and (FIG. 19I) 640 nm, respectively, taken with 0.5 mW laser power. (FIG. 19J) Corresponding ratios per pixel (FIGS. 19H-19I) mapped onto a pH scale via calibration (FIGS. 5E-5F). (FIG. 19K) Regular pixelated TIRF image in the ATTO647N reference dye channel taken with 10 mW laser power. (FIG. 19L) Binary localization mask of FIG. 19K after STORM-based emitter localization (in ThunderSTORM), intensity normalization, and pixel height binarization (see FIGS. 18A-18F). (FIG. 19M) STORM-based super-resolution-enhanced pH map without interpolation combining information via multiplication about emitter localizations from FIG. 19L with information from pixelated TIRF microcopy-based pH map in FIG. 19J (see FIGS. 18G-18K). (FIG. 19N) STORM-based super-resolution-enhanced NN-interpolated pH map transformed from FIG. 19M (see FIGS. 18G-18L).

FIGS. 20A-20B show ROI2-1 identified in a separate MDA-MB-231 TNBC cell (data not shown) incubated with aC′ dot sensor particles for 60 min. (FIG. 20A) Upper region chosen within ROI2-1 and (FIG. 20B) lower region chosen within ROI2-1 and reported pH values of 5.5 (FIG. 20A) and 5.8. (FIG. 20B) were averaged across localization areas highlighted within lines.

FIG. 21 shows an illustration of central pixel (black) in each 9×9 pixel emitter PSF area (gray).

FIGS. 22A-22B show (FIG. 22A) Histogram of number of vesicles identified with a particular average vesicle pH displayed in 5 increments of half a pH unit. This data set is based on 90 clusters identified in 11 MDA-MB-231 cells incubated with aC′ dot sensor particles for 60 min. (FIG. 22B) Scatter plot of estimates of the number of particles per vesicle as a function of vesicle diameter, and associated pH averaged across each vesicle, in 5 increments of half a pH unit. Vesicles with fewer than 25 particles, for which accurate pH values could not be determined, are displayed as open circles.

FIGS. 23A-23G show ROI1-2 from TTRF microscopy image in FIGS. 12E-12F displaying pixelated images in the FAM and ATTO647N channels with laser excitations at (FIG. 23A) 488 nm and (FIG. 23B) 640 nm, respectively, taken with 0.5 mW laser power. (FIG. 23C) Corresponding ratios per pixel (FIGS. 23A-23B) mapped onto a pH scale via calibration (FIGS. 5E-5F). (FIG. 23D) Regular pixelated TTRF image in the red reference channel taken with 10 mW laser power. (FIG. 23E) Binary localization mask of FIG. 23D after STORM-based emitter localization (in ThunderSTORM), intensity normalization, and pixel height binarization (see FIGS. 18A-18L). (FIG. 23F) STORM-based super-resolution-enhanced pH map without interpolation combining information via multiplication about emitter localizations from FIG. 23E with information from pixelated TIRF microcopy-based pH map in FIG. 23C (see FIG. 21). (FIG. 23G) STORM-based super-resolution-enhanced NN-interpolated pH map transformed from FIG. 23F (see FIG. 21).

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

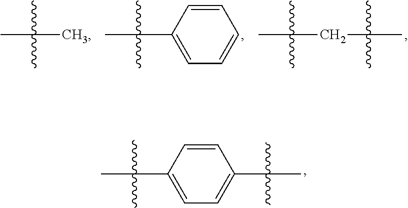

and the like. A group may also be referred to as a moiety.

The present disclosure provides methods of determining a presence or an absence of an analyte or a concentration of an analyte. The present disclosure also provides methods of treatment.

The present disclosure describes, inter alia, the use of fluorescent aluminosilicate nanoparticles, which may be ultrasmall aluminosilicate nanoparticles, comprising a reference dye encapsulated (e.g., covalently encapsulated) in the aluminosilicate core and a sensor dye attached to the aluminosilicate nanoparticle to perform ratiometric sensing enhanced by optical super-resolution microscopy (OSRM). OSRM is enabled by the particular aluminosilicate core encapsulating a fluorescent (reference) dye leading to low duty cycle blinking, which in turn enables single-molecule localization microscopy (SMLM), such as, for example, stochastic optical reconstruction microscopy (STORM). The disclosure further describes accounting for heterogeneities in the aluminosilicate nanoparticle composition encountered in typical particle synthesis efforts in order to assure that the ratiometric sensing action provides reliable results.

In an aspect, the present disclosure provides methods of determining a presence or an absence of an analyte or a concentration of an analyte in a sample or a portion thereof or an individual or a portion thereof. The presence or absence of an analyte or the concentration of an analyte are determined using one or more aluminosilicate nanoparticle(s) (which are also referred to herein as particles) in a sample or a portion thereof or an individual or a portion thereof. In any case described herein, the presence or the absence of the analyte or the concentration of the analyte may be determined locally (e.g., substantially at or at the position of one or more of the aluminosilicate nanoparticle(s) or the like) in the sample or the portion thereof or the individual or the portion thereof. In various examples, a method comprises ratiometric sensing and OSRM imaging. In. various examples, a method is an in situ method or an ex situ method. Non-limiting examples of the methods are disclosed herein.

A method can determine the presence or absence of or the concentration (which may be a local concentration) of various analytes in a sample or a portion thereof or an individual or a portion thereof using one or more aluminosilicate nanoparticle(s). In various examples, the presence or absence of or the concentration of two or more analytes in a sample or a portion thereof or an individual or a portion thereof is determined by repeating the method a desired number of times. In various examples, an analyte is a biologically relevant analyte. Non-limiting examples of biologically relevant analytes include pH, reactive oxygen species (ROS), oxidative species and/or antioxidative species, nitric oxide, metals, metal oxides, and the like. In various examples, a method determines a biologically relevant pH, a biologically relevant concentration of oxygen, a reactive oxygen species (ROS), an oxidative species and/or an antioxidative species, nitric oxide, a metal, or a metal oxide.

In various examples, the presence or absence of or the concentration of an analyte is determined using one or more aluminosilicate nanoparticle(s). In various examples, the presence or absence of or the concentration of an analyte is determined substantially at (e.g., an area in which one or more of the aluminosilicate nanoparticles interacts with the sample or a portion thereof or the individual or the portion thereof) or at the position of one or more of the aluminosilicate nanoparticle(s).

In various examples, a method comprises (or is) an OSRM enhanced ratiometric sensing method. In various examples, a method comprises (or is) an OSRM enhanced ratiometric sensing method that provides enhanced spatial resolution of one or more of the aluminosilicate nanoparticle(s). In various examples, an OSRM enhanced ratiometric sensing method provides information (e.g., the presence or absence of or the concentration of an analyte in a sample or portion thereof or an individual or portion thereof) with resolution below Abbe's diffraction limit.

In various examples, a method of determining a presence or an absence of an analyte (which may be determined locally) or a concentration (which may be a local concentration) of an analyte in a sample or a portion thereof or an individual or a portion thereof using one or more aluminosilicate nanoparticle(s) comprises contacting the sample or individual with a plurality of aluminosilicate nanoparticles; optionally, incubating the aluminosilicate nanoparticles with the sample or in the individual; determining a presence or an absence or the concentration of the analyte using ratiometric sensing at a resolution at or greater than Abbe's diffraction limit; localizing with resolution below Abbe's diffraction limit at least a portion of or all of the individual aluminosilicate nanoparticles using OSRM (or OSRM imaging); determining a presence or an absence of the analyte or the concentration of the analyte (which may be substantially at or at the position of one or more of the aluminosilicate nanoparticles) using the presence or the absence or the concentration of the analyte obtained using the ratiometric sensing and the localization of the aluminosilicate nanoparticles obtained using OSRM; and averaging the fluorescence intensity ratio of a desired number of individual aluminosilicate nanoparticles substantially at the position or at the position of an individual aluminosilicate nanoparticle to assign an average fluorescence intensity ratio to the individual aluminosilicate nanoparticle, where the average fluorescence intensity ratio assigned to the individual aluminosilicate nanoparticle(s) corresponds to the presence or the absence of the analyte or the concentration of the analyte in the sample or the portion thereof or the individual or the portion thereof.

In various examples, the determining the presence or the absence of the analyte or the concentration of the analyte is carried out using ratiometric sensing. In various examples, the fluorescence intensity ratio (which may be of one or more region(s), such as, for example, individual region(s) of an object plane) is determined using fluorescence imaging (which may be at or greater than Abbe's diffraction limit). In various examples, the fluorescence intensities are determined using a fluorescence microscope.

In various examples, determining the presence or the absence or the concentration of the analyte in an individual region of an object plane (which may be a first object plane) using ratiometric sensing is carried out by irradiating the sample or the portion thereof or the individual or the portion thereof, with a first electromagnetic energy thereby exciting at least one or all of the one or more reference dye group(s) such that the reference dye group(s) produce a first reference dye group fluorescence signal; irradiating the sample or the portion thereof or the individual or the portion thereof with a second electromagnetic energy, thereby exciting at least one or all of the one or more sensing dye group(s) such that the sensing dye group(s) produce a first sensing dye fluorescence signal; and determining a fluorescence intensity ratio of the first sensing dye fluorescence signal to the first reference dye group fluorescence signal of at least a portion of one or more or all region(s) of a detecting plane, where the fluorescence intensity ratio corresponds to a presence or an absence or a concentration of an analyte in an individual region of the object plane (which may be the first object plane).

In various examples, the determining is carried out by obtaining a fluorescence image using fluorescence emission from the sensor groups and obtaining a fluorescence image using fluorescence emission from the reference groups and determining the determining the ratio of the fluorescence intensity ratio of the sensor group (e.g., sensing dye or the like) fluorescence signal to the first reference group (e.g., reference dye group or the like) fluorescence signal. In various examples, the ratio of the fluorescence intensity ratio of the sensor group (e.g., sensing dye or the like) fluorescence signal to the first reference group (e.g., reference dye group or the like) fluorescence signal is determined for one or more portions (which may be a pixel of an optical detector) (which may be common portions) of an object plane or detector plane (e.g., pixel by pixel for an optical detector) to determine the analyte concentration via an external calibration of that fluorescence intensity signal ratio. In various examples, the fluorescence images are obtained using a fluorescence microscope.

In various examples, determining the presence or the absence or the concentration of the analyte in an individual region of an object plane (which may be a first object plane) using ratiometric sensing is carried out by determining the fluorescence intensity ratio of a fluorescence signal of the reference dye group(s) to a fluorescence signal of the sensing dye group(s), where the fluorescence intensity ratio corresponds to a presence or an absence or a concentration of an analyte in portion of the object plane.

In various examples, a method of the present disclosure provides and/or uses sub-diffraction limit resolution. Imaging methods can be referred to as super-resolution (SR) imaging methods. In various examples, an imaging method provides (e.g., exhibits) sub-diffraction limit resolution, where the diffraction limit is V/2 and X is the wavelength of the excitation light. In various examples, an imaging method provides (e.g., exhibits) a resolution 10% or less, 20% or less, or 50% or less than the diffraction limit.

Use of the one or more aluminosilicate nanoparticle(s) and/or composition(s) of the present disclosure do/does not require reducing agents as additives to an imaging buffer to provide sub-diffraction limit resolution. Accordingly, in an example, a composition used in an imaging method does not comprise an imaging buffer. Examples of imaging buffers are known in the art. Non-limiting examples of imaging buffers comprise a mixture of 2-mercaptoethanol and an enzymatic oxygen scavenger system (e.g., glucose oxidase/catalase system or the like) in phosphate-buffered saline (PBS). In an example, a composition used in an imaging method of the present disclosure does not comprise 2-mercaptoethanol or the like.

Localization with resolution below Abbe's diffraction limit at least a portion of or all of the individual nanoparticles can be carried out using OSRM (e.g., OSRM imaging methods). In various examples, the localizing with resolution below Abbe's diffraction limit at least a portion of or all of the individual nanoparticles is carried out using OSRM imaging. In various examples, the localization is in a second object plane, where the second object plane corresponds to at least a portion or all of a first object plane, which is the object plane in which the presence or the absence of the analyte or the concentration of the analyte using one or more of the aluminosilicate nanoparticles is carried out using ratiometric sensing.

Various OSRM imaging methods can be carried out using or in methods of the present disclosure. Examples of suitable OSRM methods are known in the art. Non-limiting examples of OSRM methods include ground state depletion (GSD) microscopy, stochastic optical reconstruction microscopy (STORM), direct stochastic optical reconstruction microscopy (dSTORM), stimulated emission and depletion (STED), photoactivated localization microscopy (PALM), and the like. In various examples, the localization is carried out using an OSRM imaging method described herein.

In various examples, localizing with resolution below Abbe's diffraction limit at least a portion of or all of the individual nanoparticles in a second object plane, where the second object plane corresponds to at least a portion or all of a first object plane, using OSRM imaging is carried out by irradiating the sample or the portion thereof or the individual or the portion thereof with a third electromagnetic radiation thereby exciting at least one or all of the one or more reference dye group(s) such that at least a portion of the aluminosilicate nanoparticles exhibit blinking behavior and reference dye groups produce a second reference dye group fluorescence signal; and generating an OSRM fluorescence image of at least a portion or all of the individual aluminosilicate nanoparticles in a second object plane, where the second object plane corresponds to at least a portion or all of the first object plane, using the second reference dye group fluorescence signal, where the OSRM image localizes one or more or all of the individual aluminosilicate nanoparticles with resolution below Abbe's diffraction limit.

The intensity of the electromagnetic radiation used to produce fluorescence emission for OSRM imaging may be greater than the electromagnetic radiation used to produce fluorescence emission used in the ratiometric sensing. In various examples, intensity of the electromagnetic radiation used to produce fluorescence emission for OSRM imaging is about 5 to about 15 times greater than the electromagnetic radiation used to produce fluorescence emission used in the ratiometric sensing the for OSRM.

In various examples, the determining the presence or the absence or the local concentration of the analyte in the individual region of a detecting plane using ratiometric sensing and the localizing with resolution below Abbe's diffraction limit at least a portion of or all of the individual aluminosilicate nanoparticles in the second object plane are each carried out using OSRM imaging. In these cases, at least a portion of the sensor dye(s) are covalently bonded to and encapsulated in the aluminosilicate nanoparticles (e.g., in exterior aluminosilicate shell of the aluminosilicate nanoparticles).

Determining the presence or the absence of the analyte or the concentration of the analyte using the fluorescence intensity ratio obtained using ratiometric sensing and the localization of the aluminosilicate nanoparticle(s) using OSRM imaging can be carried out in various ways. In various examples, the presence or the absence of the analyte or the concentration of the analyte is determined using the fluorescence intensity of one or more individual aluminosilicate nanoparticles.

In various examples, the determining is carried out by normalizing the OSRM image such that a particle localization is indicated by 1's while all other areas are represented by 0's, and multiplying this localization matrix with the pixel-by-pixel (e.g., at an individual pixel size of about 100 nm×about 100 nm to about 500 nm×about 500 nm or the like) of derived ratiometric analyte sensing information in order to obtain OSRM enhanced ratiometric sensing information of the sample or portion thereof or the individual or the portion thereof.

Without intending to be bound by any particular theory, it is considered that in certain cases, such as, for example, where the aluminosilicate nanoparticles exhibit inhomogeneity (e.g., inhomogeneity with regard to the number of sensor groups, reference groups, or the like, or any combination thereof), it may be desirable to correct, to account for the inhomogeneity, the concentration of the analyte assigned using the presence or the absence or the concentration of the analyte provided by the determining (e.g., determining the presence or the absence of the analyte or the local concentration of the one or more analyte substantially at or at the position of one or more of the aluminosilicate nanoparticles using the presence or the absence or the concentration of the analyte in the individual region of the first object plane and the localization of the aluminosilicate nanoparticle(s) in the second object plane). This correction can be carried out in various ways.

In various examples, the correction is carried out by averaging the fluorescence intensity ratio of a desired number of individual aluminosilicate nanoparticles substantially at or at the position of an individual aluminosilicate nanoparticle (which may be carried out using an averaging function, such as, for example, an interpolation algorithm or the like, or the like). In various examples, a method comprises averaging the fluorescence intensity ratio of a desired number of individual aluminosilicate nanoparticles substantially at or at the position of an individual aluminosilicate nanoparticle to assign an average fluorescence intensity ratio to the individual aluminosilicate nanoparticle, where the average fluorescence intensity ratio assigned to the individual aluminosilicate nanoparticle corresponds to the presence or the absence of the analyte or the local concentration of the analyte in the sample or the portion thereof or the individual or the portion thereof. In various examples, the correction is carried out using a nearest-neighbor (NN) interpolation algorithm. The number of individual aluminosilicate nanoparticles that need to be averaged (or included in the NN interpolation algorithm) can be determined by one skilled in the art. In various examples, the number (which may be the desired number) of individual aluminosilicate nanoparticles that need to be averaged (e.g., included in the NN interpolation algorithm or the like) is the number that provides a fluorescence intensity ratio substantially equal to or equal to the ensemble fluorescence intensity ratio (which may be determined by obtaining solution bulk sensor group fluorescence intensities and bulk reference groups fluorescence intensities).

An object plane, unless indicated otherwise, is a plane containing the real or virtual object in an optical system (which is typically perpendicular to the axis of the system). A detecting plane, which may be a detector (such as, for example, a detector of a fluorescence imaging system (e.g., a fluorescence microscope or the like)).

In various examples, a method comprises pH imaging of biological materials, such as, for example, cells (e.g., living cells, fixed cells, and the like), extracellular components, or tissues comprising contacting the biological material with aluminosilicate nanoparticles of the present disclosure and/or compositions comprising the aluminosilicate nanoparticles. Directing excitation electromagnetic (e/m) radiation, such as light, on to the tissues or cells thereby exciting the dye molecules; detecting e/m radiation emitted by the excited dye molecules; and capturing and processing the detected e/m radiation to provide one or more image (which may be optical super-resolution images) of the biological material. One or more steps of the method can be carried out in vitro or in vivo. For example, the cells or tissues can be present in an individual or can be present in culture. Exposure of cells or tissues to electromagnetic radiation may be affected in vitro (e.g., under culture conditions) or may be affected in vivo. For directing electromagnetic radiation at cells, extracellular materials, tissues, organs and the like within an individual or any portion of an individual's body that are not easily accessible, fiber optical instruments can be used.

In an example, a method of obtaining an image of a sample comprising a biological material comprises: contacting the sample (e.g., the individual) with one or more aluminosilicate nanoparticle(s) and/or one or more composition(s) of the present disclosure; irradiating the sample (e.g., individual or a portion thereof); optionally, an incubation period (e.g., 1 minute to 2 hours, including all 0.1 minute values and ranges therebetween); and obtaining one or more (e.g., a plurality of) fluorescence image(s) of the sample (e.g., the individual or a portion thereof). The fluorescent image(s) may be used to generate an optical super-resolution image. In another example, a method for imaging of a region within an individual comprises (a) administering to the individual one or more aluminosilicate nanoparticle(s) and/or one or more composition(s) of the present disclosure, and optionally, waiting for a selected period of time (which may be referred to as an incubation period) (e.g., 1 minute to 2 hours, including all 0.1 minute values and ranges therebetween); (b) irradiating the individual or a portion thereof with electromagnetic radiation (e.g., directing electromagnetic radiation, which may be referred to as, excitation light into the individual), thereby exciting at least one of the one or more dye molecules of the aluminosilicate nanoparticles; and obtaining one or more fluorescent image(s) of the region within the individual; (c) detecting excited light, the detected light having been emitted by the one or more pH sensing dye group(s) and/or reference dye group(s) in the individual as a result of excitation by the excitation light; and (d) processing signals corresponding to the detected light to provide one or more images (e.g., a real-time video stream), which may be one or more optical super-resolution image(s), of the region within the individual). The image may be a pH map (e.g., a ratiometric pH sensor map, a STORM imaging-based pH map, or the like). Specific pH value of one or more regions of the images may be determined.

A fluorescent image can be obtained in various ways. For example, obtaining a fluorescence image comprises: detecting excited electromagnetic radiation, the detected electromagnetic radiation having been emitted by the pH sensing dye group(s) and/or reference dye group(s) (e.g., in the sample or individual) as a result of excitation by the excitation electromagnetic radiation; and processing signals corresponding to the detected electromagnetic radiation to provide one or more fluorescent image(s) (e.g., of the sample or region within the individual), which may be used to generate one or more optical super-resolution image(s).

In an example, a method comprises obtaining a plurality of fluorescence images (e.g., about 1000 images, which may be referred to individually as frames or in the aggregate as a set), analyzing each individual frame (e.g., by localizing individual blinking events applying the point-spread function (PSF), or the like, and/or summing up over all frames and localization events to generate a super-resolution image.

The source of the electromagnetic radiation may be a laser. In various examples, the laser is a single laser.

Various aluminosilicate nanoparticles can be used in a method of the present disclosure. An aluminosilicate nanoparticle may be referred to as a nanosensor. It may be desirable that at least a portion of or all of the aluminosilicate nanoparticles exhibit blinking behavior such that the nanoparticles can be imaged using OSRM imaging. In various examples, the aluminosilicate nanoparticles are pH imaging agents or the like.

In various examples, at least a portion or all of the aluminosilicate nanoparticles are porous. In various examples, at least a portion or all of the aluminosilicate nanoparticles are microporous and/or mesoporous (according the IUPAC definition of each of these terms).

An aluminosilicate nanoparticle may be a core aluminosilicate nanoparticle (e.g., an aluminosilicate nanoparticle comprising an inner (“core”) region comprising a core composition and/or structure) or a core-shell aluminosilicate nanoparticle (e.g., a nanoparticle comprising: an inner (“core”) region comprising a core composition and/or structure and one or more outer (“shell”) region(s) disposed on at least a portion or all of the outer surface(s) (e.g., an exterior surface, a pore surface, or the like, or any combination thereof) of the inner (“core”) region, including one or more same or different shell composition(s) and/or structure(s). In various examples, an aluminosilicate nanoparticle is a PEGylated nanoparticle (e.g., a core-shell nanoparticle comprising: an inner (“core”) region comprising a core composition and/or structure and one or more outer (“PEGylated shell(s)”) comprising a shell composition and/or structure comprising polyethylene glycol groups). An aluminosilicate nanoparticle may be porous. In various examples, the core and/or aluminosilicate shell(s) are porous.

The aluminosilicate nanoparticles may each independently have an aluminosilicate core (e.g., an aluminosilicate core having an Al/Si atomic ratio of about 0.01 to about 30%, including all 0.01 at. % values and ranges therebetween) having a longest linear dimension (which may be a diameter or the like) of about 20 nm (nm=nanometer(s)) or less (e.g., less than about 20 nm, less than about 15 nm, or less than about 10 nm, or less than about 5 nm) and each aluminosilicate nanoparticle comprises a plurality of polyethylene glycol (PEG) groups disposed on (e.g., covalently bound to) at least a portion of or all of a surface (e.g., an exterior surface, a pore surface, or the like, or any combination thereof) or a portion of or all of the surfaces (e.g., exterior surface(s), pore surface(s), or the like, or any combination thereof) of the aluminosilicate nanoparticle.

An aluminosilicate nanoparticle comprises a plurality of polyethylene glycol (PEG) groups. These PEG groups may be referred to, in the alternative, as an outer (“PEGylated” shell). In various examples, aluminosilicate nanoparticles comprising a plurality of PEG groups, independently and/or on average, comprise a size (e.g., a longest linear dimension (which may be a diameter, such as, for example, a hydrodynamic diameter, a TEM diameter, or the like)) of about 20 nm or less (e.g., less than about 20 nm, less than about 15 nm, or less than about 10 nm, or less than about 5 nm).

In various examples, an aluminosilicate nanoparticle has a PEG layer that can be of various dimensions. In various examples, the chain length of the PEG groups (i.e., the molecular weight of the PEG group), individually or on average, is from about 2 EO groups to about 20 EO groups, including all integer number of EO groups and ranges therebetween (e.g., from about 5 EO groups to about 10 EO groups, or from about 6 EO groups to about 9 EO groups, including all integer number of EO groups and ranges therebetween) (e.g., about 2, about 3, about 4, about 5, about 6, about 7, about 8, about 9, about 10, about 11, about 12, about 13, about 14, about 15, about 16, about 17, about 18, about 19, or about 20 EO group(s)). The PEG chain length may be selected to tune the thickness of the PEG layer surrounding the aluminosilicate nanoparticle and the pharmaceutical kinetics profiles of the PEGylated aluminosilicate nanoparticles. The PEG chain length may be used to tune the accessibility of the ligand groups on the surface of the PEG layer of the particles resulting in varying binding and targeting performance. In various examples, PEG chain length is selected to tune the thickness of a PEG layer surrounding the aluminosilicate nanoparticle and the pharmaceutical kinetics profiles of PEGylated nanoparticles. In various examples, a PEG layer comprises various PEG group surface densities on a nanoparticle. In various examples, an aluminosilicate nanoparticle comprises a PEG group surface density of from about 1.2 PEG groups/nm² to about 2.2 PEG groups/nm², including all 0.01 PEG groups/nm² and ranges therebetween. In various examples, a PEG group is a portion of a larger/more complex group disposed on (e.g., covalently bound to or the like) a surface or a portion of or all of the surfaces of an aluminosilicate nanoparticle (e.g., via a linking group). In various examples, at least a portion of or all of the PEG groups comprise one or more ligand(s) (such PEG groups are also referred to as functionalized PEG groups). In various examples, at least a portion or all of the PEG groups comprise one or more targeting group(s), one or more diagnosing group(s), one or more therapeutic group(s), or any combination thereof.

In various examples, an aluminosilicate nanoparticle comprises (or is) an aluminosilicate core-organic ligand shell (core-organic shell) nanoparticle comprising: an aluminosilicate core, one or more reference dye group(s) covalently bound to and encapsulated in the aluminosilicate network (or matrix) of the aluminosilicate core-organic ligand shell nanoparticle, one or more sensing dye group(s) capable of analyte sensing covalently bound to the aluminosilicate core network (or matrix), where the one or more reference dye group(s) and the one or more sensing dye group(s) do not interfere with each other and/or one or more sensing dye group(s) capable of analyte sensing is/are disposed on at least a portion of or all of an surface (e.g., an exterior surface, a pore surface, or the like, or any combination thereof) or a portion of or all of the surfaces (e.g., exterior surface(s), pore surface(s), or the like, or any combination thereof) of the aluminosilicate core, and a plurality of PEG groups (which may be referred to as a shell (or organic shell) or layer) disposed on at least a portion of an surface (e.g., an exterior surface, a pore surface, or the like, or any combination thereof) or all of the surfaces (e.g., exterior surface(s), pore surface(s), or the like, or any combination thereof) of the aluminosilicate core; an aluminosilicate core-aluminosilicate shell-organic shell (core-inorganic shell-organic shell) nanoparticle comprising: an aluminosilicate core, one or more reference dye group(s) covalently bound to and encapsulated in the aluminosilicate network (or matrix) of the aluminosilicate core, an aluminosilicate shell disposed on at least a portion of a surface (e.g., an exterior surface, a pore surface, or the like, or any combination thereof) or all of the surfaces (e.g., exterior surface(s), pore surface(s), or the like, or any combination thereof) of the aluminosilicate core, one or more sensing dye group(s) capable of analyte sensing covalently bound to and encapsulated in the aluminosilicate network (or matrix) of the aluminosilicate shell, optionally, one or more sensing dye group(s) capable of analyte sensing disposed on at least a portion of or all of a surface (e.g., an exterior surface, a pore surface, or the like, or any combination thereof) or a portion of or all of the surfaces (e.g., exterior surface(s), pore surface(s), or the like, or any combination thereof) of the aluminosilicate shell, and a plurality of PEG groups (which may be referred to as a shell (or organic shell) or layer) disposed on at least a portion of an surface (e.g., an exterior surface, a pore surface, or the like, or any combination thereof) or all of the surfaces (e.g., exterior surface(s), pore surface(s), or the like, or any combination thereof) of the aluminosilicate core. In various examples, a combination of aluminosilicate core-organic ligand shell (core-organic shell) nanoparticles and aluminosilicate core-aluminosilicate shell-organic shell (core-inorganic shell-organic shell) nanoparticles are used.

Without intending to be bound by any particular theory, it is considered that the aluminosilicate nanoparticles exhibit blinking such that OSRM images can be obtained from a sample or individual. In various examples, an aluminosilicate nanoparticle comprising one or more reference dye group(s) covalently bound to and encapsulated in the aluminosilicate network (or matrix) (e.g., in an aluminosilicate core, in an aluminosilicate shell, or both) of the aluminosilicate core-organic ligand shell nanoparticle exhibit blinking that OSRM images can be obtained from a sample or individual.

It may be desirable that at least a portion of or all of the aluminosilicate nanoparticles exhibit a low duty cycle (time emitter is on/data acquisition time). In various examples, at least a portion of or all of the aluminosilicate nanoparticles exhibit a duty cycle of less than about 0.001 or less than about 0.0001.

An aluminosilicate nanoparticle (e.g., a core and aluminosilicate shell(s), if present) can be of various sizes. In various examples, the diameter of the core is about 1 nm to 50 nm, including all 0.1 nm values and ranges therebetween. In various examples, the diameter of the core is about 2 nm to about 50 nm, about 3 nm to about 50 nm, about 2 nm to about 10 nm, or about 3 nm to about 10 nm. In various examples, the thickness of the first and subsequent aluminosilicate shells, if present, are, independently, is about 1 nm to about 15 nm, in including all 0.1 nm values and ranges therebetween. In various examples, the first and subsequent shells, if present, independently are disposed on at least a portion of the surface area (e.g., about 10 percent to 100 percent), including all 0.1 percent values and ranges therebetween, of the surface area of the aluminosilicate core or subsequent shell. In various examples, the size (e.g., a longest linear dimension, such as, for example, a diameter) of an aluminosilicate nanoparticle, is from about 1 nm to 50 nm (e.g., 2 nm to about 50 nm, 3 nm to about 50 nm, , about 1 nm to about 10 nm, about 2 nm to about 10 nm, or about 3 nm to about 10 nm), including all 0.1 nm values and ranges therebetween. In various examples, the aluminosilicate nanoparticles individually have at least one dimension (which may be a longest linear dimension, such as, for example, a diameter) of 1 to 50 nm (1 nm to 50 nm (e.g., 2 nm to about 50 nm, 3 nm to about 50 nm, about 1 nm to about 10 nm, about 2 nm to about 10 nm, or about 3 nm to about 10 nm), including all 0.1 nm values and ranges therebetween.

In various examples, an aluminosilicate nanoparticle is an imaging agent (e.g., an aluminosilicate nanoparticle pH imaging agent, or the like). An imaging agent comprises an aluminosilicate nanoparticle and one or more dye group(s) capable of pH sensing disposed on (e.g., covalently bound directly or indirectly to) at least a portion of or all of a surface or a portion of or all of the surfaces of the aluminosilicate nanoparticle and one or more reference dye group(s) encapsulated by (e.g., covalently bound to) the aluminosilicate network (or matrix) of the aluminosilicate nanoparticle that may exhibit optical blinking enabling stochastic optical reconstruction microscopy (STORM). A nanoparticle may be surface functionalized with polyethylene glycol groups (e.g., PEGylated nanoparticles). A nanoparticle may be referred to as a core-shell nanoparticle or a PEGylated nanoparticle.

An aluminosilicate nanoparticle can comprise various sensor groups and reference groups. A sensor group may be referred to a sensing group. A sensor group, a reference group, or an other group, may be a dye group (e.g., derived from a dye).

In various examples, the individual reference group(s) is/are encapsulated by and optionally, covalently bound to) the aluminosilicate network (or matrix) of one or more or all of the aluminosilicate nanoparticle(s). In various examples, the individual sensor group(s) or other group(s) is/are disposed on (e.g., covalently bound to, or the like) one or more surfaces (e.g., an exterior surface, a pore surface, or the like, or any combination thereof) or encapsulated by (e.g., covalently bound to) the aluminosilicate network (or matrix) of one or more or all of the aluminosilicate nanoparticle(s). In various examples, a reference group, sensor group, or other group is covalently bound to a nanoparticle via a linking group (e.g., a fluorescent dye linked to a silane and covalently bonded to the aluminosilicate network (or matrix) via the silane). In various examples, a targeting group, a therapeutic group (e.g., a drug or the like), a diagnostic group, or the like, is a portion of a larger/more complex group (e.g., a functionalized PEG group) covalently bound to a surface or a portion of or all of the surfaces of a nanoparticle or nanoparticles (e.g., via a linking group).

A group (e.g., sensor group, reference group, or other group) can be conjugated to an aluminosilicate nanoparticle via various groups. The group(s) conjugating a dye to a nanoparticle may be part of (e.g., a group of) a group precursor used in the synthesis of the aluminosilicate nanoparticle. In various examples, a group precursor (e.g., a dye or the like) are conjugated via amino-silanes and active ester groups on the group precursor (e.g., a dye or the like). In various examples, the group precursor (e.g., a dye or the like) are not conjugated via mercapto-silanes and maleimido groups on the group precursor (e.g., a dye or the like). In various examples, a sensor group is indirectly conjugated to a surface (e.g., an external surface or a pore surface) of an aluminosilicate nanoparticle via a PEG group (which may be referred to as a PEG linking group).

Methods of forming covalently bonded groups are known in the art. In various, examples, one or more group(s) are formed by post-PEGylation surface modification by insertion (PPSMI).

In various examples, an individual aluminosilicate nanoparticle comprises from about 1 to about 5, including all integer values and ranges therebetween, (e.g., about 2, about 3, about 4, or about 5) types of different groups, where at least two of the groups are a sensor group and a reference group.

An aluminosilicate nanoparticle can comprise various sensor dyes (e.g., pH sensing dyes, or the like, or any combination thereof) and reference dyes. The dyes may be organic dyes. An aluminosilicate nanoparticle can comprise various pH sensing and reference dyes. The dyes may be organic dyes. In an example, a dye does not comprise a metal atom. A dye may be conjugated to an aluminosilicate nanoparticle. In various examples, a dye is conjugated to an aluminosilicate nanoparticle forming a dye group.

A dye group capable of pH sensing or a reference dye group can be derived from a dye molecule. A dye group may be covalently bound directly or indirectly (e.g., via a linking group) a surface of an aluminosilicate nanoparticle via a PEG group. A dye can be conjugated to an aluminosilicate nanoparticle via various groups. The group(s) conjugating a dye to a nanoparticle may be part of (e.g., a group of) a dye precursor used in the synthesis of the aluminosilicate nanoparticle. In various examples, the dyes are conjugated via amino-silanes and active ester groups on the dye. In various examples, the dyes are not conjugates via mercapto-silanes and maleimido groups on the dye. In various examples, a dye group capable of pH sensing is indirectly conjugated to a surface of an aluminosilicate nanoparticle via a PEG group (which may be referred to as a PEG linking group).

In various examples, an aluminosilicate nanoparticle comprises various dyes (e.g., groups formed from various dyes). In various examples, at least a portion of the aluminosilicate nanoparticles have a dye or combination of dyes (e.g., a NIR dye) encapsulated therein. In various examples, the dye groups (which may be formed from dye molecules) are covalently bound to the aluminosilicate nanoparticles (e.g., where the dye groups are at least partially or completely encapsulated by, covalently bound to the aluminosilicate network (or matrix), or the like and/or disposed on the aluminosilicate nanoparticle surface). In various examples, the dyes are organic dyes. In an example, a dye does not comprise a metal atom. Non-limiting examples of dyes include fluorescent dyes (e.g., near infrared (NIR) dyes and the like), phosphorescent dyes, non-fluorescent dyes (e.g., non-fluorescent dyes exhibiting less than 1% fluorescence quantum yield), fluorescent proteins (e.g., EBFP2 (variant of blue fluorescent protein), mCFP (Cyan fluorescent protein), GFP (green fluorescent protein), mCherry (variant of red fluorescent protein), iRFP720 (Near Infra-Red fluorescent protein)), and the like, and groups derived therefrom. In various examples, a dye absorbs in the UV-visible portion of the electromagnetic spectrum. In various examples, a dye has an excitation and/or emission in the near-infrared portion of the electromagnetic spectrum (e.g., 650-900 nm).

Non-limiting examples of organic dyes include cyanine dyes (e.g., Cy5®, Cy3®, Cy5.5®, Cy7®, and the like), carborhodamine dyes (e.g., ATTO 647N (available from ATTO-TEC and Sigma Aldrich®), BODIPY dyes (e.g., BODIPY 650/665 and the like), xanthene dyes (e.g., fluorescein dyes such as, for example, fluorescein isothiocyanate (FITC), Rose Bengal, and the like), eosins (e.g. Eosin Y and the like), and rhodamines (e.g. TAMRA, tetramethylrhodamine (TMR), TRITC, DyLight® 633, Alexa 633, HiLyte 594, and the like), Dyomics® DY800, Dyomics® DY782 and IRDye® 800CW, and the like, and groups derived therefrom.

A nanoparticle can comprise various sensor groups. A sensor group may be referred to as a ligand. The fluorescence of a sensor group is altered by the presence of an analyte (e.g., interaction of the sensor group with the analyte). Non-limiting examples of sensor groups include pH sensing groups, ion sensing groups, oxygen sensing groups, biomolecule sensing groups, temperature sensing groups, and the like, and any combination thereof.

In various examples, a sensor group or sensor groups is/are pH sensing dye group(s). In various examples, the pH sensing dye group(s) comprise(s) (or is/are derived from) fluorescein isothiocyanate, β-5-carboxyfluorescein-bis-(5-carboxymethoxy-2-nitrobenzyl) ether-alanine-carboxamide-succinimidyl ester, 5-carboxy-2′,7′-dichlorosulfonefluorescein, carboxy seminaphthofluorescein (SNAFL-1)—NHS ester, Oregon Green Carboxylic Acid—NHS ester, fluorescein, 2′,7′-bis-(2-carboxyethyl)-carboxyfluorescein, Seminapthorhodafluor-1 (SNARF-1), 4′,5′-dichloro-2′,7′ dimethoxyfluorescein, Resorufin ethers (benzyl, ethyl, methyl, etc.), or the like, or any combination thereof.

In various examples, a sensor group or sensor groups is/are redox status sensing dye group(s). In various examples, a redox sensing dye group senses ratio or balance of oxidant(s) and antioxidant(s). In various examples, the redox sensing dye group(s) comprise(s) (or is/are derived from) dihydrofluorescein derivatives (such as, for example, dichlorodihydrofluorescein (DCFH₂), OxyBURST (H₂HFF), and the like), dihydrorhodamine, and hydroethidiene (HE), folate-(BODIPY FL)-SS-rhodamine reporter (folate-FRET), BODIPY FL L-cysteine, di-(o-aminobenzyl) glutathione disulfide (diabz-GSSG), or TAMRA disulfide dimer (ssTAMBA), or the like, or any combination thereof.

In various examples, a sensor group or sensor groups is/are oxygen sensing dye group(s). In various examples, the oxygen sensing dye group(s) comprise(s) (or is/are derived from) Erythrosin B isothiocyanate, or the like, or any combination thereof.

In various examples, a sensor group or sensor groups is/are reactive oxygen species sensing dye group(s). In various examples, the reactive oxygen species sensing dye group(s) comprise(s) (or is/are derived from) boron dipyrromethene (BODIPY 665-676), or the like, and or combination thereof.

In various examples, a sensor group or sensor groups is/are chloride ion sensing dye group(s). In various examples, the chloride ion sensing dye group(s) comprise(s) (or is/are derived from) 6-methoxy-N-(3-sulfopropyl)quinolinium), or the like, or any combination thereof.

In various examples, a sensor group or sensor groups is/are nitric oxide sensing dye group(s). In various examples, the nitric oxide sensing dye group(s) comprise(s) (or is/are derived from) diaminonapthalene, 4,5-diaminofluorescein, 4-amino-5-methylamino 2′,7′-difluorofluorescein, dihydrorhodamine, or the like, and any combination thereof.

In various examples, a sensor group or sensor groups is/are metal or metal ion sensing dye group(s). In various examples, the metal or metal ion sensing dye group(s) comprise(s) (or is/are derived from) fluo-4 iodoacetamide, Fluo-4, Calcium Green, X-Rhod, PBFI (spectral Shift), Zinquin Ethyl Ester, Calcium Yellow, Magnesium Green, Calcium Orange, CoroNa Red, RhodZin-3, sulforhodamineamidoethyl mercaptan, or the like, or any combination thereof.

An aluminosilicate nanoparticle can comprise various reference groups (e.g., reference dye groups, or the like). A reference group may be referred to as a ligand.

In various examples, a reference group is/are reference dye group(s). In various examples, the reference dye group(s) comprise(s) (or is/are derived from) Molecular Probes—AlexaFluor 350, Molecular Probes—Pacific Blue, Molecular Probes—AlexaFluor 488, Molecular Probes—AlexaFluor 532, Rhodamine B Isothiocyanate, Tetramethylrhodamine—Isothiocyanate, Molecular Probes—AlexaFluor 568, Dyomics DY 610, Dyomics DY 615, Molecular Probes—AlexaFluor 647, Dyomics DY 675, Dyomics DY 700, Dyomics DY 731, Dyomics DY 776, Sigma Aldrich—NIR 797, Dyomics DY 485 XL, Dyomics DY 510 XL, or the like, or any combination thereof.

Non-limiting examples of dyes include fluorescent dyes, fluorescent proteins (e.g., EBFP2 (variant of blue fluorescent protein), mCFP (Cyan fluorescent protein), GFP (green fluorescent protein), mCherry (variant of red fluorescent protein), iRFP720 (Near Infra-Red fluorescent protein)), and the like. In various examples, a dye absorbs in the UV-visible portion of the electromagnetic spectrum. In various examples, a dye has an excitation and/or emission in the near-infrared portion of the electromagnetic spectrum (e.g., 650-900 nm).

Various examples of sensor groups and reference groups are known in the art. Non-limiting examples of sensor groups and reference groups are described in U.S. Pat. No. 8,084,001 (Photoluminescent silica-based sensors and methods of use, filed May 2, 2005), the disclosure of which with regard to sensor groups, reference groups, and methods of forming same are incorporated herein by reference. In various examples, at least a portion or all of the sensor group(s) and/or reference group(s) is that/are those described in and/or made by a method disclosed in this U.S. Patent.

One or more or all of the aluminosilicate nanoparticles may comprise one or more other group(s) (which are not or do not comprise sensor group(s) and reference group(s)). In various examples, the other (non-sensor and non-reference group(s)) are chosen from targeting groups, therapeutic groups (e.g., a drug group or the like), and diagnostic groups, and the like, and any combination thereof, any of which may be referred to as a functional group, a ligand, or the like. In various examples, an other group is disposed on (e.g., covalently bound to, or the like) one or more surfaces (e.g., an exterior surface, a pore surface, or the like, or any combination thereof) or encapsulated by (e.g., covalently bound to) the aluminosilicate network (or matrix) of one or more or all of nanoparticle(s). In various examples, a ligand, such as, for example, a dye group or the like, is at least partially or completely encapsulated by and/or covalently bound the aluminosilicate network (or matrix) of a nanoparticle. In various examples, a ligand is covalently bound to a nanoparticle via a linking group (e.g., a fluorescent dye linked to a silane and covalently bonded to aluminosilicate network (or matrix) via the silane). In various examples, a targeting group, a therapeutic group (e.g., a drug or the like), a diagnostic group, or the like, is a portion of a larger/more complex group (e.g., a functionalized PEG group) covalently bound to a surface or a portion of or all of the surfaces of a nanoparticle or nanoparticles (e.g., via a linking group).

An aluminosilicate nanoparticle may comprise one or more targeting group(s). A targeting group can allow (or facilitate) targeted delivery of an aluminosilicate nanoparticle or aluminosilicate nanoparticles. A targeting group may be formed from (derived from) a targeting molecule, biological material, or the like. In various examples, a targeting group derived from a targeting molecule, biological material, or the like has substantially the same properties (e.g., activity, which may be biological activity or the like) as the targeting molecule, biological material, or the like from which it is derived. In various examples, a targeting group, which is capable of binding to a cellular component (e.g., on the cell membrane or in the intracellular compartment) associated with a specific cell type, is conjugated to the aluminosilicate nanoparticle. The targeting group may be a tumor marker or a molecule in a signaling pathway. The targeting group may have specific binding affinity to certain cell types, such as, for example, tumor cells. In certain examples, the targeting group may be used for guiding the aluminosilicate nanoparticles to specific areas, such as, for example, liver, spleen, brain or the like. Imaging can be used to determine the location of the aluminosilicate nanoparticles in an individual. Non-limiting examples of targeting groups include groups with targeting ability (e.g., antibody fragments, aptamers, proteins/peptides (natural, truncated, or synthetic), nucleic acids such as, for example, DNA and RNA, and the like). Non-limiting examples of targeting groups include linear and cyclic peptides (e.g., integrin-targeting cyclic(arginine-glycine-aspartic acid-D-tyrosine-cysteine) peptides, c(RGDyC), and the like), antibody fragments, various DNA and RNA segments (e.g. siRNA). Other non-limiting examples of targeting groups include cancer-targeting peptides, and the like, and any combination thereof.

An aluminosilicate nanoparticle may comprise various targeting groups. Non-limiting examples of targeting groups include groups with targeting ability (e.g., antibody fragments, aptamers, proteins/peptides (natural, truncated, or synthetic), nucleic acids such as, for example, DNA and RNA, and the like). Non-limiting examples of targeting groups include linear and cyclic peptides (e.g., integrin-targeting cyclic(arginine-glycine-aspartic acid-D-tyrosine-cysteine) peptides, c(RGDyC), and the like), antibody fragments, various DNA and RNA segments (e.g. siRNA). Other non-limiting examples of targeting groups include cancer-targeting peptides, and the like, and any combination thereof.

An aluminosilicate nanoparticle may comprise various diagnostic groups. In various examples, a diagnostic group provides diagnostic information about an individual. A diagnostic group may be formed from (derived from) a molecule, an atom, a biological material, or the like. Non-limiting examples of diagnostic groups include groups having absorption/emission behavior such as, for example, fluorescence and phosphorescence, which in various examples is used for imaging, sensing functionality (e.g., pH sensing, ion sensing, oxygen sensing, biomolecules sensing, temperature sensing, and the like), or the like. In various examples, a diagnostic group is chosen from dye groups, sensor groups, radioisotopes, and the like, and any combination thereof. In various examples, imaging is used to determine the location of the aluminosilicate nanoparticles in an individual.

In various examples, an other group (such as, for example, a therapeutic group, a diagnostic group, or the like) comprises a radioisotope. In various examples, a radioisotope is a diagnostic agent and/or a therapeutic agent. For example, a radioisotope, such as for example, ¹²⁴I, is used for positron emission tomography (PET), and the like. Non-limiting examples of radioisotopes include ³H, ¹⁴C, ¹⁸F, ¹⁹F, ³²p, ³⁵S, ¹³⁵I, ¹²⁵I, ¹²⁴I, ¹²³I, ¹³¹I, ⁶⁴Cu, ⁶⁸Ga, ¹⁸⁷Re, ¹¹¹In, ⁹⁰Y, ^{99m}Tc, ¹⁷⁷Lu, ⁸⁹Zr, as well as radiotherapeutic isotopes, such as, for example, ²²⁵Ac, ¹⁷⁷Lu, and the like. A radioisotope may be chelated to a chelating group.

An aluminosilicate nanoparticle may comprise various chelator groups. Non-limiting examples of chelator groups include desferoxamine (DFO), 1,4,7,10-Tetraazacyclododecane-1,4,7,10-tetraacetic acid (DOTA), 1,4,7-triazacyclononane-1,4,7-triacetic acid (NOTA), ethylenediaminetetraacetic acid (EDTA), diethylenetriaminepentaacetic acid (DTPA), porphyrins, and the like, and groups derived therefrom. A chelator group may comprise a radioisotope. Non-limiting examples of radioisotopes are described herein and suitable examples of radioisotopes are known in the art.

An aluminosilicate nanoparticle may comprise various therapeutic groups. As referred to herein, unless otherwise stated, a therapeutic group is defined as any molecule, atom, or the like, or any combination thereof, with therapeutic ability (e.g., drugs (which may be small molecule drugs and the like), nucleic acids, biological materials, radioisotopes, and the like, and any combination thereof). In various examples, a therapeutic group is formed from (derived from) a molecule, atom, or the like with therapeutic ability. In various examples, a therapeutic group releases a therapeutic agent (which may be the native form or an active form of a drug, nucleic acid, or the like) from a nanoparticle having substantially all (e.g., at least 90%, at least 95%, or at least 99% of the parent drug's activity) or all of the native (e.g., unconjugated form of the drug, nucleic acid, or the like) drug's, nucleic acid's, or the like's activity. In various examples, a therapeutic group is formed from a drug (which may be a small molecule drug), a nucleic acid, or the like.

A group may have both imaging and therapeutic functionality. In various examples, a group having both imaging and therapeutic functionality is formed from a compound or radioisotope exhibiting imaging and therapeutic functionality by derivatization of the compound and/or radioisotope using conjugation chemistry and reactions known in the art.

Non-limiting examples of therapeutic agents include, but are not limited to, chemotherapeutic agents, small molecule inhibitors, cytotoxic drugs, antibiotics, antifungal agents, antiparasitic agents, antiviral agents, anti-inflammatory agents, neurological agents, psychotherapy agents, groups comprising one or more radiotherapeutic isotope(s) (such as, for example, ²²⁵Ac, ¹⁷⁷Lu, and the like), and the like, and any combination thereof. Any of these agents may be drugs (e.g., drugs, which may be small molecule drugs and the like, nucleic acids, biological materials, radioisotopes, and the like). In various examples, a therapeutic group is formed from (derived from) one of these therapeutic agents.

In various examples, a therapeutic group is a drug group. A variety of drugs (e.g., small molecule drugs and the like) can be used to form a drug group. In various examples, a drug disposed on is conjugated to a surface of a nanoparticle. Drugs can be conjugated to a surface of a nanoparticle by methods known in the art. A drug group may release a drug from a nanoparticle having substantially all (e.g., at least 90%, at least 95%, or at least 99% of the parent drug's activity) or all of the parent drug's activity.

In various examples, a therapeutic group (e.g., a drug group, such as, for example, a drug-linker conjugate group, where the linker group is capable of being specifically cleaved by enzyme or acid condition in tumor for drug release, is disposed (e.g., covalently bonded to) a surface of a nanoparticle (e.g., attached to a functional ligand on a surface of a nanoparticle) for drug delivery. In various examples, drug-linker-thiol conjugates are attached to maleimido-PEG-particles through thiol-maleimido conjugation reaction post the synthesis of maleimido-PEG-particles.

It may be desirable to form a drug group from a hydrophobic drug. In various examples, a drug group is a hydrophobic drug group. Therapeutic groups may be formed from (e.g., derived from) therapeutic agents (e.g., drugs, which may be small molecule drugs, such as, for example, small molecule inhibitors, cytotoxic drugs, and the like, and the like), nucleic acids, biological materials, radioisotopes, and the like), and the like, that are not considered amenable to oral administration.

Suitable aluminosilicate nanoparticles are known in the art. In various examples, the aluminosilicate nanoparticles are aluminosilicate Cornell dots (e.g., aC dots or aC′ dots), which are PEGylated. Non-limiting examples of aluminosilicate nanoparticles are described in U.S. Published Patent Application Nos. 20180133346 (Ultrasmall nanoparticles and methods of making and using same, filed May 4, 2016), 20190282712 (Inhibitor-functionalized ultrasmall nanoparticles and methods thereof, filed Nov. 29, 2017), 20200101180 (Ultrasmall nanoparticles labeled with zirconium-89 and methods thereof, filed May 27, 2018), 20200179538 (Functionalized nanoparticles and methods of making and using same, filed May 21, 2018), 20200316219 (Methods of treatment using ultrasmall nanoparticles to induce cell death of nutrient-deprived cancer cells via ferroptosis, filed Jun. 16, 2020), 20210048414 (Ultrasmall nanoparticles and methods of making, using and analyzing same, filed May 2, 2019), and International Publication No. WO 2017/189961 (Compositions and methods for targeted particle penetration, distribution, and response in malignant brain tumors, filed Apr. 28, 2017), U.S. Pat. No. 10,732,115 (Mesoporous oxide nanoparticles and methods of making and using same, filed Jun. 6, 2013), the disclosure of which with regard to aluminosilicate nanoparticles and methods of making same are incorporated herein by reference. In various examples, the aluminosilicate nanoparticle(s) is that/are those described in and/or made by a method disclosed in one or more of these U.S. Published Patent Applications and/or U.S. Patent. In various examples, a composition comprises one or more aluminosilicate nanoparticle(s) described in and/or made by a method disclosed in one or more of these U.S. Published Patent Application(s) and/or U.S. Patent(s).

The aluminosilicate nanoparticles may be contacted with the sample or the portion thereof or the individual thereof as a composition. A composition comprises a plurality of aluminosilicate nanoparticles. In various examples, all of the aluminosilicate nanoparticles are the same. In various examples, two or more of the aluminosilicate nanoparticles are different.

A composition can comprise two or more type(s) of aluminosilicate nanoparticles (e.g., having different average size and/or one or more different compositional feature(s)). In various examples, a composition comprises a plurality of aluminosilicate core nanoparticles, aluminosilicate core-shell nanoparticles, or the like, or any combination thereof. Any of the aluminosilicate nanoparticles may be surface functionalized with one or more type(s), size(s), surface densit(ies), or the like, of polyethylene glycol groups (e.g., polyethylene glycol groups, functionalized (e.g., functionalized with one or more ligand(s) and/or reactive group(s)) polyethylene glycol groups, or any combination thereof). In various examples, one or more of the aluminosilicate nanoparticle(s) has/have at least one structural and/or compositional feature (e.g., core structure (e.g., core, core-shell, or the like), core composition (e.g., Al/Si ratio or the like), encapsulated group concentration (e.g., dye or the like), surface group composition (e.g., type (e.g., PEG group, targeting group, therapeutic group, diagnostic group, or the like), size, surface density, or the like)) different that one or more or all of the other nanoparticles.

The aluminosilicate nanoparticles in a composition can have a variety of sizes. In an example, the aluminosilicate nanoparticles, independently, have a core size of 1 nm to 50 nm (e.g., 3 to 20 nm, 3 to 10 nm), including all 0.1 nm values and ranges therebetween. In various examples, the aluminosilicate nanoparticles, independently, have a size of 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 nm. In various examples, at least 90%, 95%, 96%, 97%, 98%, 99%, 99.5% 99.9%, or 100% of the aluminosilicate nanoparticles have a size (e.g., a longest linear dimension) of 1 nm to 50 nm (e.g., 2 to 20 nm, 3 to 20 nm, 3 to 10 nm). In various examples, at least 90%, 95%, 96%, 97%, 98%, 99%, 99.5% 99.9%, or 100% of the aluminosilicate nanoparticles have a core size (e.g., a longest linear dimension) of 1 nm to 50 nm (e.g., 2 to 20 nm, 3 to 20 nm, 3 to 10 nm).

In various examples, the aluminosilicate nanoparticles in a composition have a variety of sizes (e.g., a longest linear dimension, which may be a diameter, or the like (e.g., a hydrodynamic diameter, a TEM diameter, or the like)) and/or size distributions. In various examples, the aluminosilicate nanoparticles, independently, have a size (e.g., a core size, a core-shell size, a size including PEG groups, a size excluding PEG groups, a size for each nanoparticle independently, an average size for a plurality of nanoparticles, or the like, or any combination thereof) of about 50 nm or less (e.g., about 20 nm or less, about 15 nm or less, about 10 nm or less, or about 5 nm or less). In various examples, the aluminosilicate nanoparticles, independently, have a size (e.g., a core size, a core-shell size, a size including PEG groups, a size excluding PEG groups, a size for each nanoparticle independently, an average size for a plurality of nanoparticles, or the like, or any combination thereof) of from about 1 nm to about 50 nm (e.g., from about 1 nm to about 20 nm, about 2 nm to about 20 nm, about 3 nm to about 20 nm, about 1 nm to about 15 nm, about 2 nm to about 15 nm, about 3 nm to about 15 nm, about 1 nm to about 10 nm, about 2 nm to about 10 nm, about 10 nm), including all 0.1 nm values and ranges therebetween. In various examples, the aluminosilicate nanoparticles have a size (e.g., a core size, a core-shell size, a size including PEG groups, a size excluding PEG groups, a size for each nanoparticle independently, an average size for a plurality of nanoparticles, or the like, or any combination thereof) of about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, or about 20 nm. In various examples, at least about 90%, about 95%, about 96%, about 97%, about 98%, about 99%, about 99.5% about 99.9%, or about 100% of the aluminosilicate nanoparticles have a size (e.g., a core size, a core-shell size, a size including PEG groups, a size excluding PEG groups, a size for each nanoparticle independently, an average size for a plurality of nanoparticles, or the like, or any combination thereof) of from about from about 1 nm to about 50 nm (e.g., from about 1 nm to about 20 nm, about 2 nm to about 20 nm, about 3 nm to about 20 nm, about 1 nm to about 15 nm, about 2 nm to about 15 nm, about 3 nm to about 15 nm, about 1 nm to about 10 nm, about 2 nm to about 10 nm, about 10 nm), including all 0.1 nm values and ranges therebetween. In various examples, at least a portion of or all of the aluminosilicate nanoparticles) used in a method or in a composition have size and/or size distribution described in this paragraph.

For the exemplary particle size distributions of nanoparticles described herein, the composition may not be subjected to any particle-size discriminating (particle size selection/removal) processes (e.g., filtration, dialysis, chromatography (e.g., GPC), centrifugation, etc.). The aluminosilicate nanoparticles may be as synthesized and not have any post-synthesis processing/treatment. For the exemplary particle size distributions described herein, the composition may be subjected to one or more purification step(s) in which larger particle aggregates, smaller chemical reagents, or the like, or any combination thereof, are separated from nanoparticles.

The aluminosilicate nanoparticles may have a narrow particle size distribution. In various examples, the particle size distribution of the aluminosilicate nanoparticles, not including extraneous materials, such as, for example, aggregates, unreacted reagents, dust particles/aggregates, is +/−5, 10, 15, or 20% of the average particle size (e.g., a longest linear dimension).

Particle size and distribution (e.g., a core size/distribution, a core-shell size/distribution, a size/distribution including PEG groups, a size/distribution excluding PEG groups, a size for each nanoparticle independently, an average size/distribution for a plurality of nanoparticles, or the like, or any combination thereof) can be determined by methods known in the art. In various examples, a particle size is determined by chromatography (e.g., gel permeation chromatography or the like), spectroscopy (e.g., dynamic light scattering (DLS), fluorescence correlation spectroscopy (FCS), or the like), electron microscopy (e.g., transmission electron microscopy (TEM), scanning electron microscopy (SEM), or the like) or the like. DLS contains systematic deviation and, therefore, the DLS size distribution may not correlate with the particle size distribution determined by TEM or GPC.

A composition can comprise one or more types (e.g., having different average size and/or one or more different compositional feature) of aluminosilicate nanoparticle pH imaging agents. In various examples, all of the aluminosilicate nanoparticle pH imaging agents comprising a composition are the same. In various other examples, at least a portion of the aluminosilicate nanoparticle pH imaging agents are different e.g., having different average size and/or one or more different compositional feature). Any of the aluminosilicate nanoparticles may be surface functionalized with one or more type of polyethylene glycol groups (e.g., polyethylene glycol groups, functionalized (e.g., functionalized with one or more ligand and/or a reactive group) polyethylene glycol groups, or any combination thereof). Any of the aluminosilicate nanoparticles can have a dye or combination of dyes (e.g., a NIR dye) encapsulated therein. The dye molecules are covalently bound to the aluminosilicate nanoparticles. The aluminosilicate nanoparticles can be made by a method of the present disclosure.

The aluminosilicate nanoparticles may have a narrow size distribution. In various examples, the aluminosilicate nanoparticle size distribution, not including extraneous materials, such as, for example, aggregates, unreacted reagents, dust particles/aggregates, is +/−5, 10, 15, or 20% of the average particle size (e.g., a longest linear dimension). The particle size can be determined by methods known in the art. For example, the particle size is determined by TEM, GPS, or DLS. DLS contains systematic deviation and, therefore, the DLS size distribution may not correlate with the size distribution determined by TEM or GPS.

The composition can comprise additional component(s). As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Some non-limiting examples of materials which can be used as additional material(s) in a composition include sugars, such as, for example, lactose, glucose, sucrose, and the like; starches, such as, for example, corn starch, potato starch, and the like; cellulose, and its derivatives, such as, for example, sodium carboxymethyl cellulose, ethyl cellulose, cellulose acetate, and the like; powdered tragacanth; malt; gelatin; talc; excipients, such as, for example, cocoa butter, suppository waxes, and the like; oils, such as, for example, peanut oil, cottonseed oil, safflower oil, sesame oil, olive oil, corn oil, soybean oil, and the like; glycols, such as, for example, propylene glycol and the like; polyols, such as, for example, glycerin, sorbitol, mannitol, polyethylene glycol, and the like; esters, such as, for example, ethyl oleate, ethyl laurate, and the like; agar; buffering agents, such as, for example, magnesium hydroxide, aluminum hydroxide, and the like; alginic acid; pyrogen-free water; isotonic saline; Ringer's solution; ethyl alcohol; phosphate buffer solutions; and other non-toxic compatible substances employed in pharmaceutical formulations. (See, e.g., REMINGTON'S PHARM. SCI., 15^{th} Ed. (Mack Publ. Co., Easton (1975)).

Aluminosilicate nanoparticle(s) and/or composition(s) comprising the present nanoparticles can be administered to an individual by any suitable route-either alone or as in combination with other agents. Administration can be accomplished by any means, such as, for example, by parenteral, mucosal, pulmonary, topical, catheter-based, or oral means of delivery. Parenteral delivery can include, for example, subcutaneous, intravenous, intramuscular, intra-arterial, and injection into the tissue of an organ. Mucosal delivery can include, for example, intranasal delivery. Pulmonary delivery can include inhalation of the agent. Catheter-based delivery can include delivery by iontophoretic catheter-based delivery. Oral delivery can include delivery of an enteric coated pill, or administration of a liquid by mouth. Transdermal delivery can include delivery via the use of dermal patches.

Following administration of aluminosilicate nanoparticles or a composition comprising the aluminosilicate nanoparticles, the path, location, and clearance of the aluminosilicate nanoparticles may be monitored using one or more imaging techniques of the present disclosure. In various examples, the spatial and/or temporal distribution of the aluminosilicate nanoparticles in a sample or one or more portion(s) thereof.

Methods of the present disclosure can be used on various samples. In various examples, a sample is a cell or cells (e.g., living cells, fixed cells, and the like), extracellular components, or tissues comprising contacting the biological material, or the like, or a portion thereof.

Methods of the present disclosure can be used on various individuals or a portion thereof. In various examples, an individual is a human or non-human mammal. Examples of non-human mammals include, but are not limited to, farm animals, such as, for example, cows, hogs, sheep, and the like, as well as pet or sport animals such as horses, dogs, cats, and the like. Additional non-limiting examples of individuals include rabbits, rats, mice, and the like. The aluminosilicate nanoparticles or compositions comprising nanoparticles can be administered to individuals for example, in pharmaceutically acceptable carriers, which facilitate transporting the aluminosilicate nanoparticles from one organ or portion of the body to another organ or portion of the body. In various examples, a portion of an individual is a biopsy sample or a resected tissue sample.

In an aspect, the present disclosure provides method of treatment. The methods of treatment comprise a method of determining a presence or an absence of an analyte or a concentration (which may be a local concentration) of an analyte with one or more aluminosilicate nanoparticle(s) in a sample or a portion thereof or an individual or a portion thereof of the present disclosure. Non-limiting examples of methods of treatment are disclosed herein.

In various examples, additionally or alternatively, radioisotopes are further attached to the ligand groups (e.g., tyrosine residue or chelator) of the ligand-functionalized aluminosilicate nanoparticles or to the aluminosilicate network (or matrix) of the PEGylated aluminosilicate particles without specific ligand functionalization for positron emission tomography (PET) imaging. If the radioisotopes are chosen to be therapeutic, such as, for example, ²²⁵Ac, ¹⁷⁷Lu, or the like, this in turn would result in particles with additional radiotherapeutic properties.

A method may further comprise one or more additional (or other) therapeutic modalit(ies). Non-liming examples of therapeutic modalities include conventional/traditional drug therapies, surgical intervention (e.g., one or more surgical procedure(s) and the like), chemotherapy, radiation, and the like. A method may further comprise one or more additional (or other) diagnostic modalit(ies). Non-liming examples of diagnostic modalities include conventional/traditional diagnostic tests, methods, or the like. In various examples, the diagnostic modality is an imaging method (e.g., CT imaging, MRI, PET, x-ray imaging, or the like), or the like. In various examples, the additional modalit(ies) is/are carried out before, after, or in concert with a method of the present disclosure. As fluorescent aluminosilicate nanoparticles are typically brighter than free dye, these fluorescent nanoparticles can be used, for example, for tissue imaging and tumor (e.g., metastatic tumor) imaging.

“Treating” or “treatment” of any disease or disorder refers, in various examples, to ameliorating the disease, disease state, condition, disorder, side effect, or the like, or any combination thereof, (e.g., arresting, reversing, alleviating, or the like) the disease, disease state, condition, disorder, side effect, or the like, or any combination thereof, or reducing the manifestation, extent or severity of one or more clinical symptom(s) thereof, or the like). In various other examples, “treating” or “treatment” refers to ameliorating one or more physical parameter(s), which, independently, may or may not be discernible by the individual. In yet other examples, “treating” or “treatment” refers to modulating disease, disease state, condition, disorder, side effect, or the like, or any combination thereof, either physically, (e.g., stabilization of one or more discernible symptom(s), or the like), physiologically, (e.g., stabilization of one or more physical parameter(s), or the like), or both. In yet other examples, treating” or “treatment” relates to slowing the progression of the disease, disease state, condition, disorder, side effect, or the like, or any combination thereof.

In various examples, a method of the present disclosure targets, diagnoses, treats, prevents, or the like, or any combination thereof, any current or potential condition, disease, disease state, or the like, or any combination thereof, that may be conventionally or traditionally targeted, diagnosed, treated, or prevented, or the like, or any combination thereof, with a targeting agent, therapeutic agent, diagnosing agent, or the like, or any combination thereof, that can be delivered using one or more composition(s) of the present disclosure. Non-limiting examples of diseases, disease states, conditions, disorders, side effects, and the like, and potential diseases, disease states, conditions, disorders, side effects, and the like, include infections (e.g., bacterial infections, viral infections, and the like), cancers, neurological conditions/diseases, neurodegenerative diseases, psychological conditions/diseases, inflammatory conditions/diseases, cardio-vascular diseases, and the like, and any combination thereof.

The following Statements describe various examples of methods of the present disclosure and are not intended to be in any way limiting:

Statement 1. A method of obtaining an optical super-resolution image (e.g., an optical image or the like with resolution below Abbe's diffraction limit) of a sample or a portion thereof or an individual or a portion thereof comprising: contacting the sample (which may be a biological material) or individual with a one or more (e.g., a plurality of) aluminosilicate nanoparticle(s) of the present disclosure (e.g., aluminosilicate nanoparticle(s) comprising one or more dye group(s) capable of pH sensing, which may be disposed on (e.g., covalently bound to) at least a portion of or all of a surface or a portion of or all of the surfaces of the aluminosilicate nanoparticle and one or more reference dye group(s), which may be covalently bound to the aluminosilicate network (or matrix) of the individual aluminosilicate nanoparticles) or a composition comprising the one or more (e.g., plurality) of aluminosilicate nanoparticles; optionally, an incubation period (e.g., 1 minute to 2 hours, including all 0.1 minute values and ranges therebetween); and irradiating the sample or a portion thereof or the individual or a portion thereof, thereby exciting at least one of the dye molecules of a nanoparticle of the composition; and obtaining a fluorescence image (which may be a super resolution image) or a sequence of fluorescence images which can be processed to obtain a super resolution image (which may be a super resolution optical image) (e.g., a pH map, such as, for example, a ratiometric pH sensor map, a STORM imaging-based pH map, or the like, which may be a super resolution optical image) of the sample or portion thereof or the individual or a portion thereof.
Statement 2. A method according to claim 1, wherein the obtaining the fluorescence image comprises: detecting excited electromagnetic radiation, the detected electromagnetic radiation having been emitted by the dye molecules in the individual particle as a result of excitation by the excitation electromagnetic radiation; and processing signals corresponding to the detected electromagnetic radiation to provide one or more fluorescence images (which may be optical super-resolution images) of the sample or portion thereof or the individual or a portion thereof.
Statement 3. A method according to claim 1 or claim 2, wherein at least a portion of the optical super-resolution image exhibits sub-diffraction limit resolution.
Statement 4. A method according to any one of the preceding claims, wherein the method is an optical super-resolution microcopy method including, but not limited to, ground state depletion (GSD) microscopy, stochastic optical reconstruction microscopy (STORM), direct stochastic optical reconstruction microscopy (dSTORM), stimulated emission and depletion (STED), or photoactivated localization microscopy (PALM).
Statement 5. A method according to claim 1, wherein the contacting is administering the composition to the individual.
Statement 6. A method according to claim 5, wherein the electromagnetic radiation is directed into the individual.
Statement 7. A method according to claim 6, wherein the electromagnetic radiation is directed into a region, wherein the region is within the individual.
Statement 8. A method according to any one of the preceding claims, wherein the electromagnetic radiation comprises one or more wavelengths from 400 to 1200 nm, including all nm values and ranges therebetween.
Statement 9. A method according to any one of the preceding claims, wherein the irradiation is carried out using a laser (e.g., a single laser).
10. A method according to any one of the preceding claims, wherein the electromagnetic radiation is a single wavelength.
Statement 11. A method according to any one of the preceding claims, wherein the one or more dye group(s) capable of pH sensing and/or reference dye groups comprise (or are derived from) a fluorescent dye or fluorescent protein.
Statement 12. A method according to any one of the preceding claims, wherein the one or more dye group(s) capable of pH sensing and/or reference dye groups comprise (or are derived from) cyanine dyes, rhodamine dyes (e.g., carborhodamine dyes and the like), coumarin dyes, boron-dipyrromethene (BODIPY) dyes, xanthene dyes, eosin dyes, carbopyronine dyes, methylene blue, fluorescein, Acridine Orange, and a group/groups derived therefrom, and any combination thereof.
Statement 13. A method according to any one of the preceding claims, wherein the aluminosilicate nanoparticles individually have at least one dimension (which may be a longest linear dimension, such as, for example, a diameter) of 1 to 30 nm, including all 0.1 nm values and ranges therebetween.
Statement 14. A method of treating an individual for cancer comprising: obtaining an image of a sample (e.g., a biological material) or a portion thereof or an individual or a portion thereof according to any one of claims 1-13. A method may further comprise any treatments (e.g., one or more additional step) typically used in treatment of cancer. The individual treatment(s) (e.g., the one or more additional step(s)) may be carried out before and/or after and/or during obtaining the image. In various examples, a method further comprises one or more chemotherapy treatment(s), one or more radiation treatment(s), one or more photodynamic therapy treatment(s), one or more surgical intervention(s) (e.g., surgical procedure(s), or the like), or the like, or a combination thereof. In various examples, the sample is a biopsy obtained from an individual.
Statement 15. A method according to claim 14, wherein the method further comprises visualization of the abnormal cells after administration of the nanoparticle or the composition.
Statement 16. A method according to claim 15, wherein the visualization is carried out using fluorescence imaging.
Statement 17. A method according to any one of the preceding claims, wherein the method further comprises administration of a chemotherapy agent.
Statement 18. A method according to any one of the preceding claims, wherein the method further comprises surgical removal of at least a portion of a cancerous tissue from the individual.
Statement 19. A method according to any one of the preceding claims, wherein the method further comprises subjecting the individual to a radiation treatment.
Statement 20. A kit comprising one or more (e.g., a plurality of) aluminosilicate nanoparticles and/or a composition comprising the aluminosilicate nanoparticle(s), and instructions for use of composition(s) for obtaining an image of a sample or a portion thereof or an individual or a portion thereof and/or treatment of an individual for cancer.

The steps of the methods described in the various embodiments and examples disclosed herein are sufficient carry out the methods of the present disclosure. Thus, in an embodiment, a method consists essentially of any combination of the steps of the methods disclosed herein. In another embodiment, a method consists of such steps.

The following example is presented to illustrate the present disclosure. It is not intended to be limiting in any manner.

### Example 1

This Example provides a description of aluminosilicate nanoparticle pH imaging agents and compositions comprising the agents of the present disclosure. The Example also provides uses of the aluminosilicate nanoparticle pH imaging agents and the compositions comprising the agents.

The interrogation of metabolic parameters like pH in live-cell experiments using optical super-resolution microscopy (OSRM) remains challenging. This is due to a paucity of appropriate metabolic probes enabling live-cell OSRM-based sensing. Herein is introduced ultrasmall fluorescent core-shell aluminosilicate nanoparticle sensors (FAM-ATTO647N aC′ dots) that covalently encapsulate a reference dye (ATTO647N) in the core and a pH-sensing moiety (FAM) in the shell. Only the reference dye exhibits optical blinking enabling live-cell stochastic optical reconstruction microscopy (STORM). Using data from cells incubated for 60 minutes with FAM-ATTO647N aC′ dots, pixelated information from total internal reflection fluorescence (TIRF) microscopy-based ratiometric sensing can be combined with that from STORM-based localizations via the blinking reference dye in order to enhance the resolution of ratiometric pH sensor maps beyond the optical diffraction limit. A nearest-neighbor interpolation methodology is developed to quantitatively address particle compositional heterogeneity as determined by separate single-particle fluorescence imaging methods. When combined with STORM-based estimates of the number of particles per vesicle, vesicle size, and vesicular motion as a whole, this analysis provides detailed live-cell spatial and functional information, paving the way to a comprehensive mapping and understanding of the spatiotemporal evolution of nanoparticle processing by cells important, e.g., for applications in nanomedicine.

Ultrasmall (sub-10 nm), bright, and stable live-cell-STORM-enabling aluminosilicate nanoparticles, or aC′ dots, are an aluminosilicate derivative of regular aqueous sol-gel silica-derived Cornell prime dots, or C′ dots, ultrasmall fluorescent dye-encapsulating silica core—poly(ethylene glycol) (PEG) shell (core-shell) nanoparticles. The core formation, PEGylation, and surface functionalization mechanisms as well as physico-chemical and photophysical properties of C′ dots have been extensively characterized. As a result of their favorable in vivo biodistribution and pharmacokinetic profiles, ultrasmall C′ dots have been the first optical inorganic nanoparticle probes to obtain investigational new drug (IND) Food and Drug Administration (FDA) approval for human clinical trials, a significant milestone of this work. In addition to diagnostics and drug delivery applications in oncology, in recent studies C′ dots have also shown self-therapeutic properties without the attachment of cytotoxic agents, a new frontier in nanoparticle-based therapeutics. Understanding C′ dots' cellular trafficking and processing is therefore crucial to elucidating their fundamental mechanisms of action in order to further improve their performance.

Adding aluminum alkoxides into the synthesis of standard C′ dots to form aC′ dots leads to incorporation of 4-fold coordinated aluminum into the silica network of the core. In turn, as demonstrated in a detailed recent study, this drives photo-induced redox blinking, with aluminum in the silica network likely acting as a reducing agent, and dissolved oxygen in the imaging solution as an oxidizing agent. Unlike in standard photoswitching-inducing setups, this optical probe behavior enables live-cell STORM by only requiring a single (excitation) laser and a simple imaging buffer, without the need for a second UV laser and cytotoxic imaging cocktails. Furthermore, quantitative analysis of live-cell STORM-imaging data allows estimations of both the sizes of intracellular vesicles containing aC′ dots as well as the number of aC′ dots in each vesicle. Finally, a broad range of organic dyes across different dye families encapsulated in aC′ dots exhibit substantial fluorescence intensity as well as photostability enhancements relative to their parent free dyes in aqueous solutions. All these characteristics make the aC′ dot a desirable candidate for the development of a live-cell ratiometric sensing platform, as it simultaneously overcomes the aforementioned issues of low spatial resolution, signal-to-noise ratio, photostability, as well as biocompatibility.

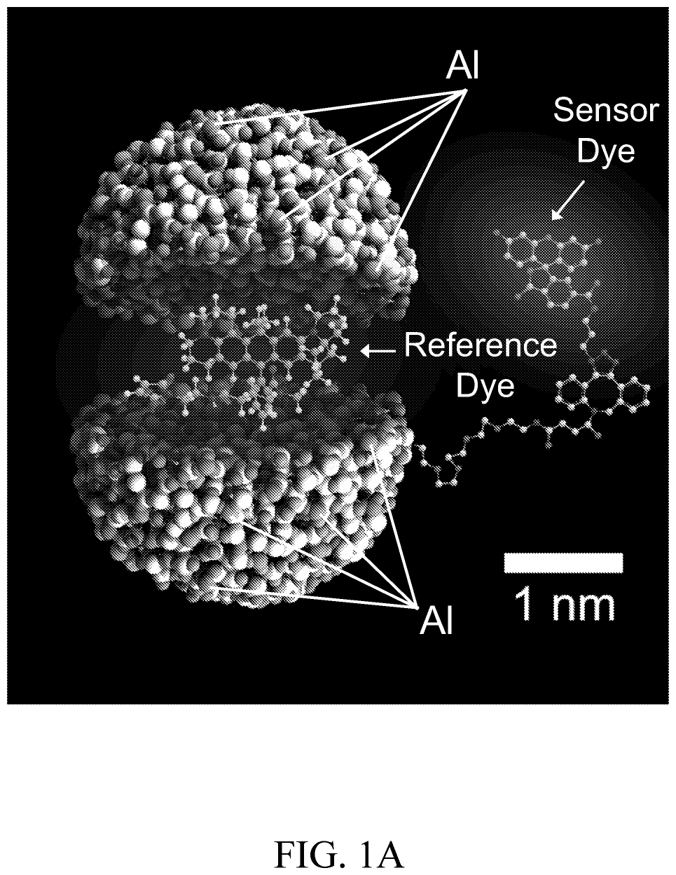

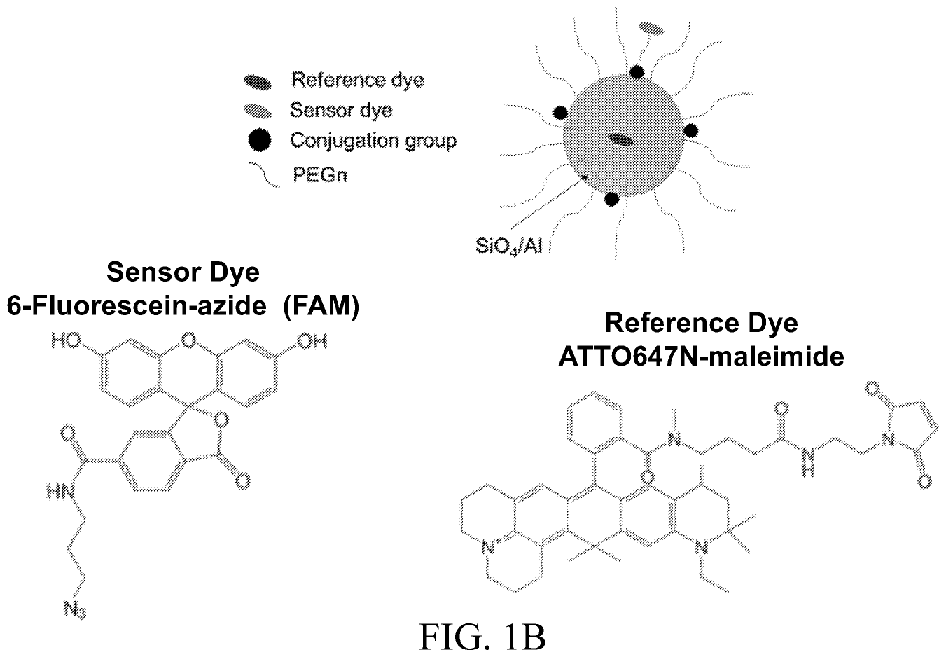

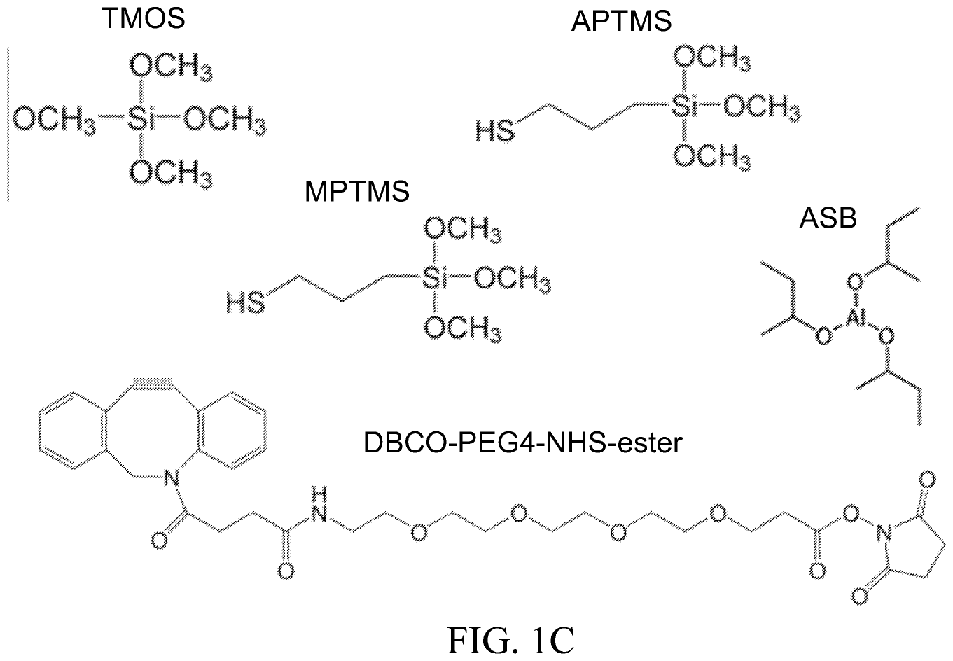

Building on the discovery of the STORM-enabling blinking properties of aC′ dots, herein is described the concept of a super-resolution-enhanced nanosensor (FAM-ATTO647N aC′ dot) that contains additional functionalities in the PEG shell enabling live-cell fluorescent ratiometric sensing aided by STORM. The first realization of this nanosensor concept in the form of a super-resolution-enhanced ratiometric pH sensor adds a pH-sensitive dye to the PEG periphery of the blinking aC′ dots (FIG. 1). This enables live-cell investigations of local nanoparticle pH environments at near-super-resolution using a standard total internal reflection fluorescence (TTRF) microscope setup without the need for any additional imaging cocktails containing cytotoxic ingredients. To that end, after their synthesis aC′ dots are first surface functionalized with a well-known pH-sensing moiety, FAM, a fluorescein-amidite dye derivative. As these nanosensors are synthesized via regular solution synthesis methods, compositional heterogeneities are typically expected in the number of reference and sensor dyes present across individual particles of a specific particle synthesis batch. In this Example, such compositional heterogeneities were quantitatively accounted for in localized ratiometric sensing experiments. To that end, these heterogeneities were first quantitatively characterized using single-particle fluorescence imaging to determine the minimum number of particles required in a local area to be averaged over in order to achieve pH detection accuracy as compared to ensemble averaged measurements. Furthermore, combining this information with STORM-based spatial probe localization information, an image processing methodology was then developed that enhances the resolution of diffraction-limited ratiometric pH sensing data and accounts for single particle sensing heterogeneity to accurately approximate the pH at each localization. For proof-of-principle experiments, high-resolution pH sensing results were produced from MDA-MB-231 triple negative breast cancer (TNBC) cells incubated with nanosensors for 60 minutes. These were then combined with aforementioned quantitative analyses of STORM data toward identification of the sizes of intracellular vesicles containing aC′ dots, the number of aC′ dots in each vesicle, as well as vesicular motion as a whole. With the ability to approximate the number of aC′ dots within a single vesicle, the heterogeneity information was used to provide localized pH measurements with high accuracy. As shown herein, this combination based on high-resolution imaging data analysis provides a wealth of detailed information about the local environment of the probes that goes well beyond e.g., what was previously accessible with the first diffraction-limited fluorescent silica core-shell nanoparticle-based ratiometric C dot sensors (synthesized in ethanol) developed by Applicant more than a decade ago. In order to optimize the synthetic conditions and establish similar cellular trafficking behavior between aC′ dot sensors developed here and the clinically-translated C′ dot platform (i.e. without aluminum in the core), a diffraction-limited nanosensor (FAM-ATTO647N C′ dot) was also synthesized and the time evolution of its in situ cellular pH conditions studied using diffraction-limited laser scanning confocal microscopy. Finally, in the conclusion section the significance of the results is discussed in the context of nanosensor probe developments in general, as well as for the detailed elucidation of the spatiotemporal evolution of cellular processing of nanoparticles in particular.

pH Nanosensor Synthesis and Characterization. A super-resolution-enhanced pH nanosensor was formed, aC′Dot(ATTO647N)-PEG6-9_amine-NHS-PEG4-DCBO-azide-FAM, hereon referred to as FAM-ATTO647N aC′ dot. To that end, using published protocols an azide-functionalized pH-sensing fluorescein dye (FAM-azide or FAM) was attached via click chemistry to dibenzocyclooctyne (DBCO)-surface-functionalized, ATTO647N-encapsulating, and PEGylated aC′ dots (aC′Dot(ATTO647N)-PEG6-9_amine-NHS-PEG4-DBCO). A rendering of the ATTO647N-encapsulating aluminosilicate particle core with the FAM sensing moiety attached to the core surface as well as the chemical structures of all synthesis components are shown in FIG. 1. ATTO647N aC′ dots were first synthesized using a water-based aluminosilicate sol-gel process as described herein. In short, after sol-gel aluminosilicate cores were formed in water at acidic pH from tetramethylorthosilicate (TMOS), aluminum-tri-see-butoxide (ASB), and ATTO647N dye-silane conjugate (fluorescent reference dye) precursors, PEG-silane (6-9 EO units) was added in the PEGylation step providing steric nanoparticle stabilization. Sensor FAM dyes were then attached to the aluminosilicate core surface of the PEGylated aC′ dots using a method referred to as post-PEGylation surface modification by insertion (PPSMI). This method ensures that surface modifications with functional moieties are performed after full steric particle stabilization via PEGylation in aqueous solution, thereby decoupling particle stabilization from further particle surface functionalization. To that end, small functional silanes are first inserted in-between the PEG chains and covalently attached to the inorganic core surface, followed by reactions with complementary functionalized moieties. In this work, PPSMI was accomplished via DBCO-azide click chemistry. An amino-silane, aminopropyl-trimethoxysilane (APTMS), was first conjugated to the aluminosilicate core surface of PEGylated aC′ dots and then reacted with a N-hydroxysuccinimidyl-ester (NHS-ester) and DBCO-bearing heterobifunctional NHS-PEG4-DBCO linker. The strained alkyne group of DBCO subsequently enabled efficient click chemistry with the complementary azide group on the FAM sensor dye (FIG. 1). In addition to the aC′ dot pH sensor, an identical C′ dot pH sensor, but with the regular sol-gel-derived silica core instead of the aluminosilicate core (hereon referred to as FAM-ATTO647N C′ dot), was synthesized to be used as a non-super-resolution-capable control probe as described herein.

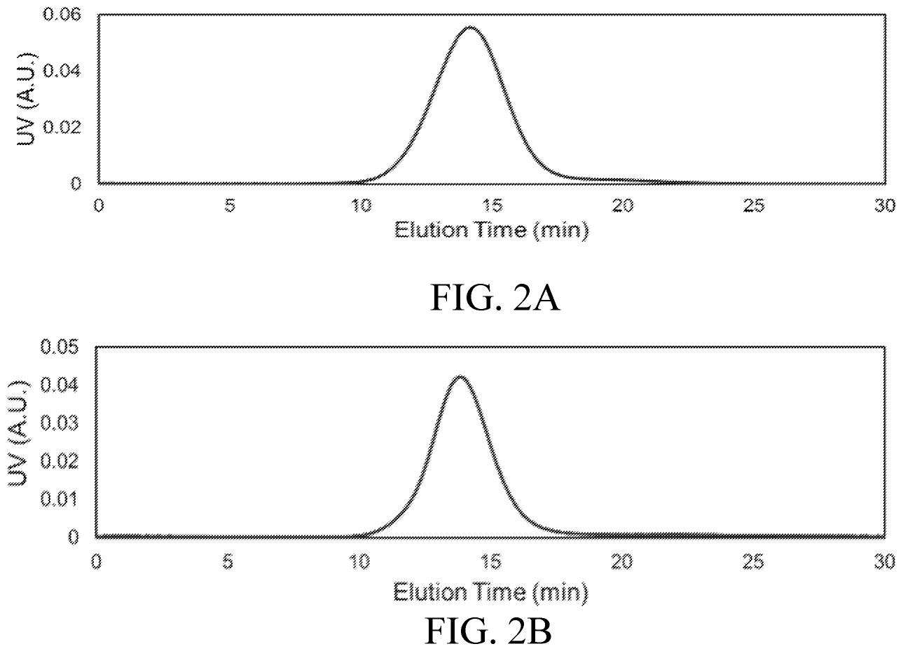

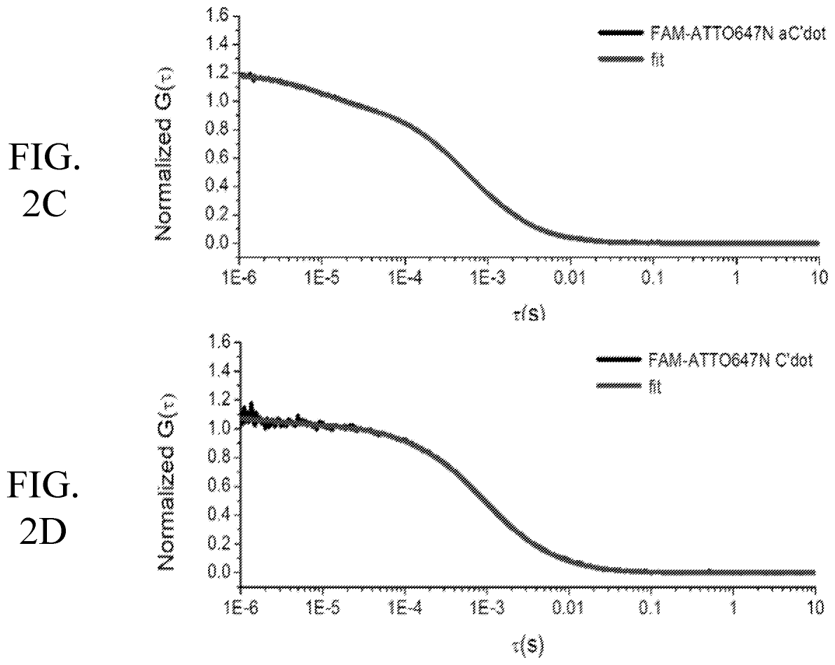

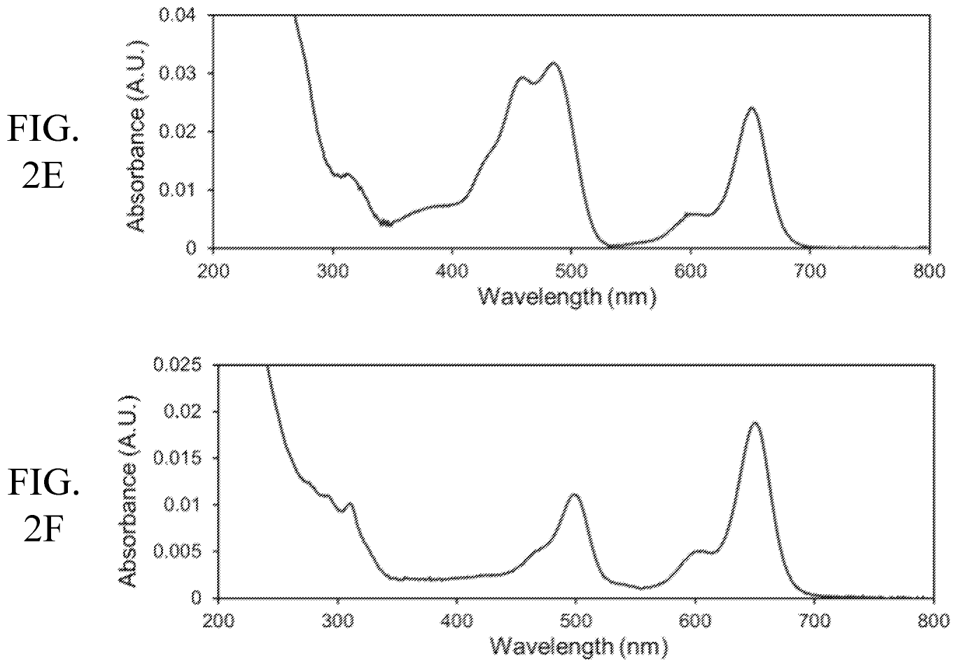

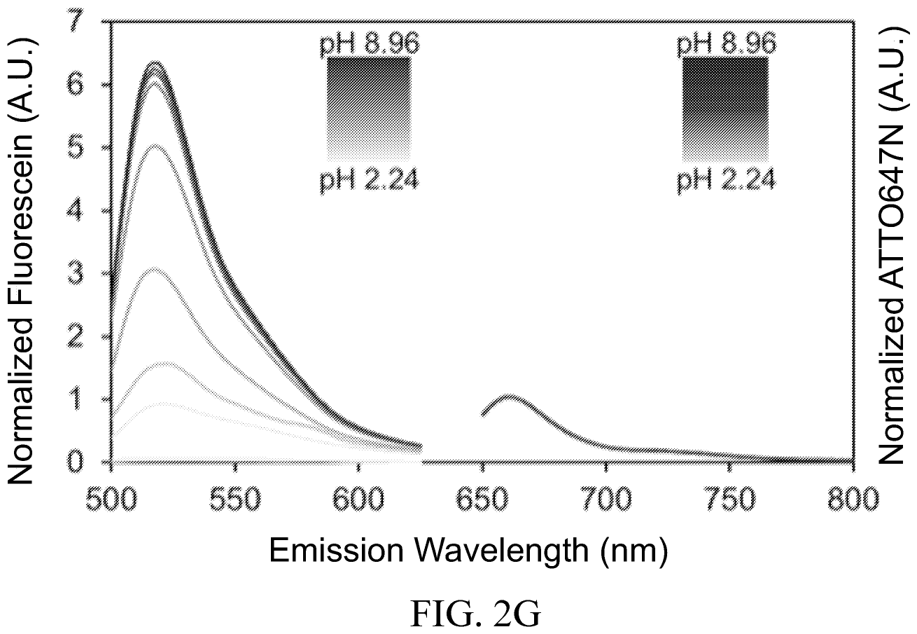

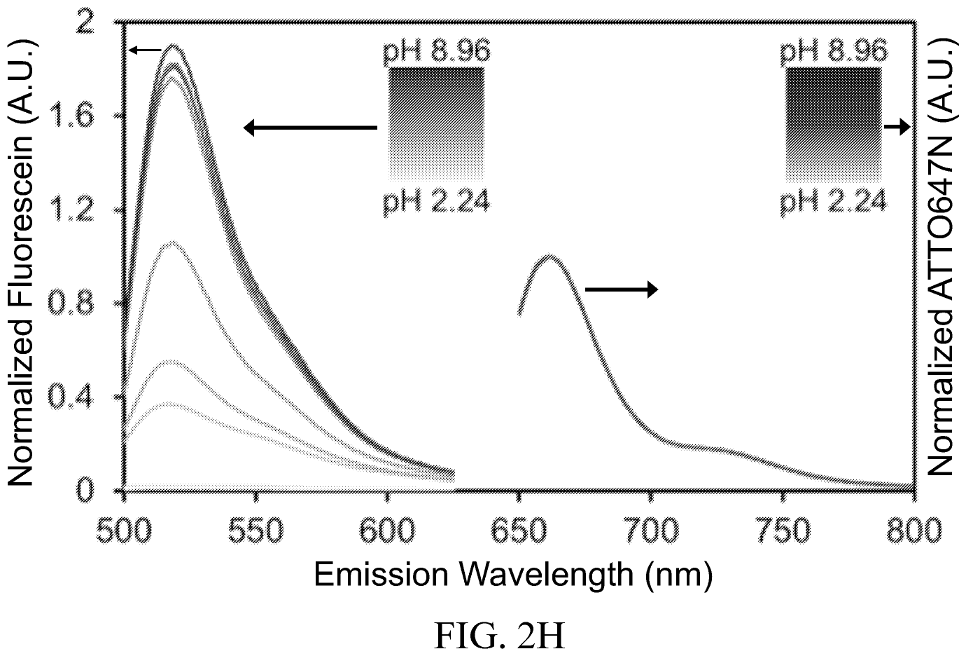

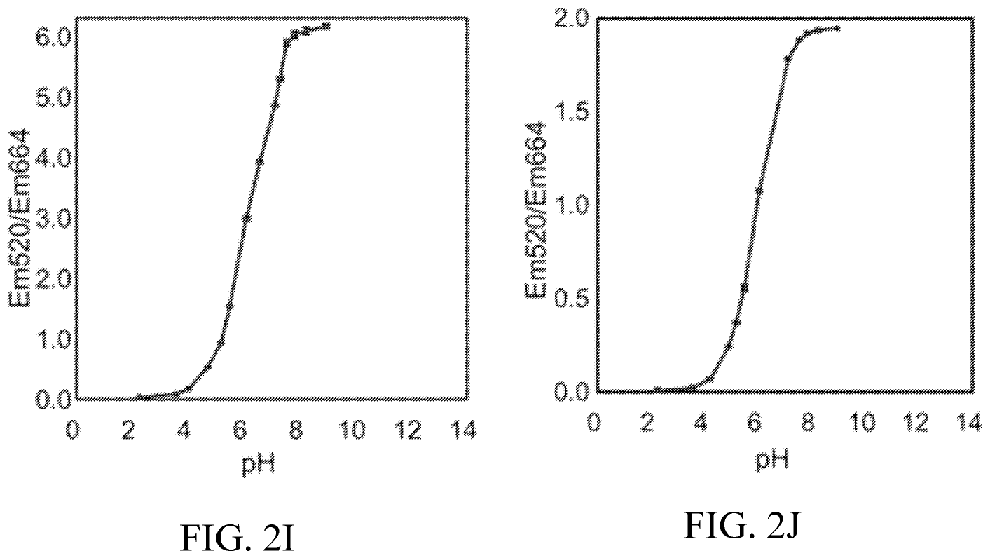

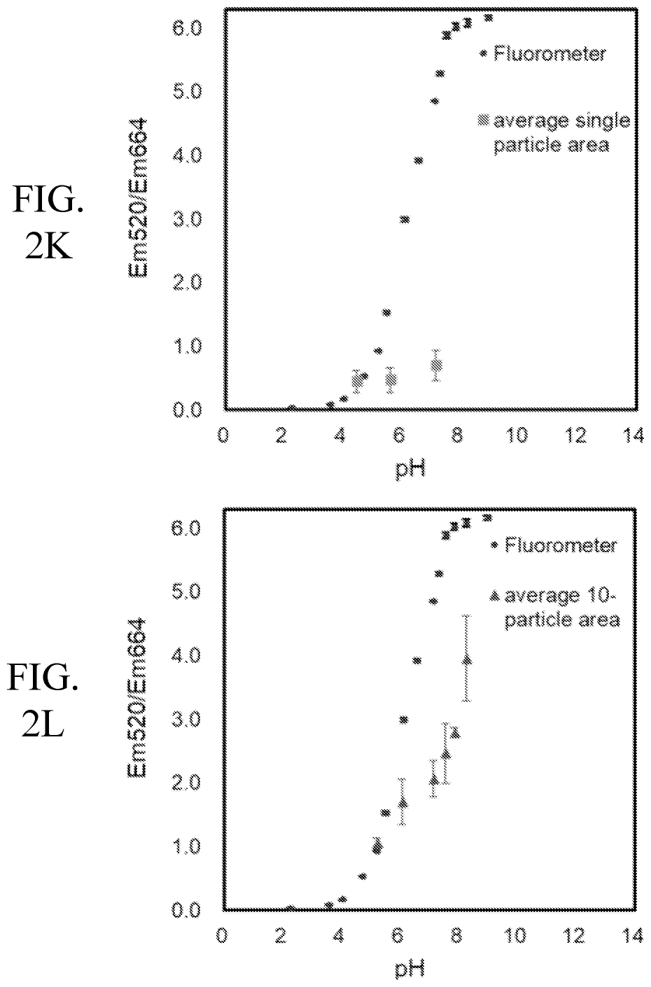

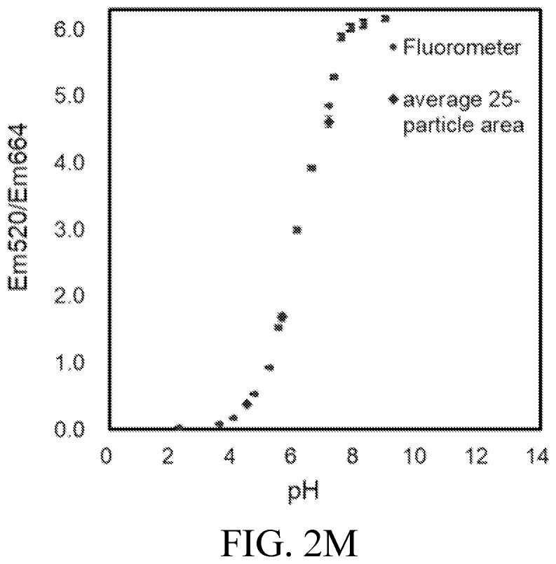

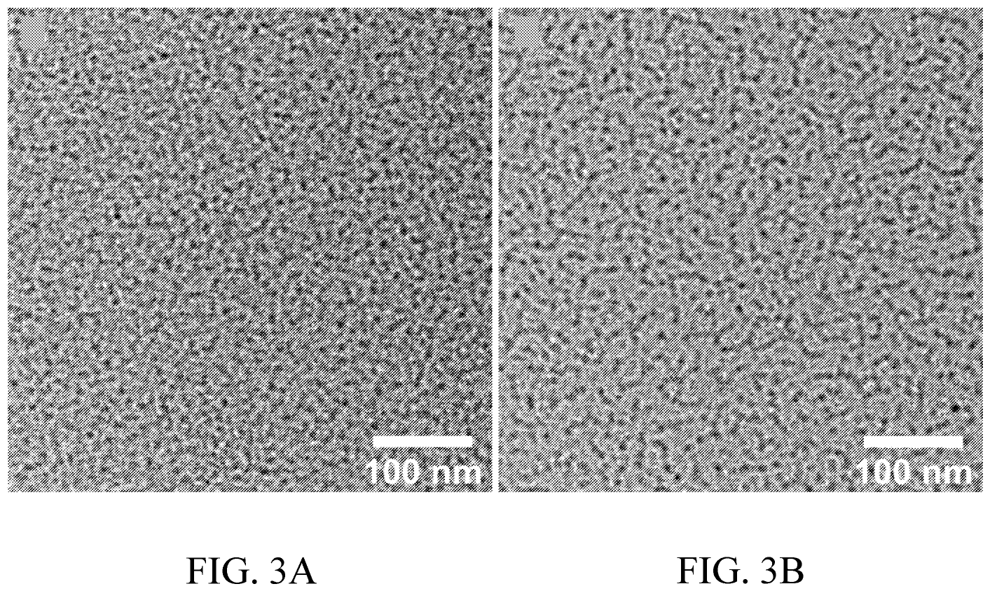

Following established protocols, GPC was used to purify the resulting nanosensors from residual precursors as well as particle aggregates, ensuring narrow size distributions. For particle characterization, FCS in combination with UV-Vis spectroscopy were subsequently used following established protocols to determine hydrodynamic size, number of dyes per particle, and concentration of the nanosensors. After purification, for both aC′ dot and C′ dot sensors, second GPC runs showed a single peak with a Gaussian-like elution profile (FIGS. 2A-2B) suggesting high quality particle batches, while FCS analysis revealed hydrodynamic diameters of 4.8 nm and 6.7 nm, respectively (FIGS. 2C-2D). TEM microscopy confirmed size uniformity and dispersion in aqueous solutions for both particle batches (FIGS. 3A-3B). Combining UV-Vis spectroscopy results with the concentration data obtained from FCS (FIGS. 2E-2F), the average numbers of both reference and sensor dyes per nanosensor were determined. For aC′ dot sensors an average of 1 ATTO647N dye and 2.5 FAM dyes per particle were deduced, while for C′ dot control sensors those average numbers were 1.4 ATTO647N dyes and 1.6 FAM dyes per particle. DBCO has a marked absorbance signature at 291 nm that decreases when the molecule is reacted with an azide. This behavior indirectly confirmed FAM dye attachment to each nanosensor (data not shown).

The number of 2.5 FAM sensor dyes per aC′ dot was higher than the 1.6 FAM dyes per C′ dot. Together with the smaller size of 4.8 nm versus 6.7 nm for aC′ dots versus C′ dots, this likely is responsible for the enhanced blue shifted FAM absorption shoulder observed for aC′ dots when comparing absorption spectra in FIGS. 2E-2F. This shoulder commonly corresponds to self-quenching of fluorescein dyes, consistent with more FAM dyes on a smaller particle having a higher probability of close proximity. For proof-of-principle experiments reported herein, sensor dye numbers per aC′ dot particle were not further optimized, although this is clearly possible.

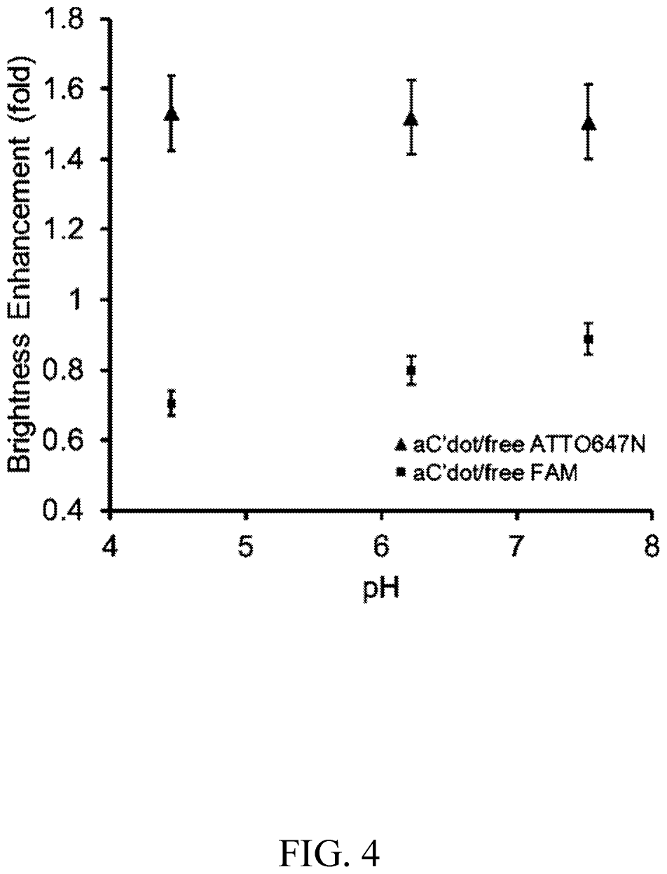

Relative brightness per dye in aC′ dots as compared to free dyes (ATTO647N and FAM) in solution was evaluated by measuring their respective absorbance and emission maxima in PBS solutions at various pH values as described herein. The brightness per ATTO647N in aC′ dots was enhanced on average about 1.5 fold, essentially independent of pH (FIG. 4). This enhancement is a result of the rigidification by the aluminosilicate matrix, as investigated in detail earlier, but was slightly reduced compared to values obtained in previous studies in ultrapure 18.2 MO water, likely due to the PBS buffer. When the same measurements were performed for ATTO647N in ultrapure water, the brightness enhancement indeed increased to 1.73+0.11, which is within error from what was previously reported. Relative brightness at pH values of 4.5, 6.2, and 7.5 for FAM in aC′ dots increased from 0.71 to 0.80 and to 0.89, respectively (FIG. 4). This pH dependent increase in brightness is the basis for the ratiometric pH sensing discussed below. In contrast to ATTO647N, the brightness of FAM in aC′ dots was reduced compared to free dye. This observation is due to two effects: First, the sensor dye is not encapsulated in the aluminosilicate core, but associated with the C dot surface (see FIG. 1) and therefore does not benefit from the same degree of rigidification as the reference dye ATTO647N. Second, as discussed before, there is self-quenching of FAM dyes as a result of their proximity, as evidenced by the corresponding shoulder in FIG. 2E.

Due to instrument limitations, zeta potential measurements were not performed on the nanosensors. The red absorption of the nanosensors strongly interfered with the red excitation laser of the instrument rendering quantitative measurements impossible. Previous studies have repeatedly demonstrated, however, that as a result of the PEG shell the zeta-potential of these particles, for both C′ dots as well as aC′ dots, is at or close to neutral.

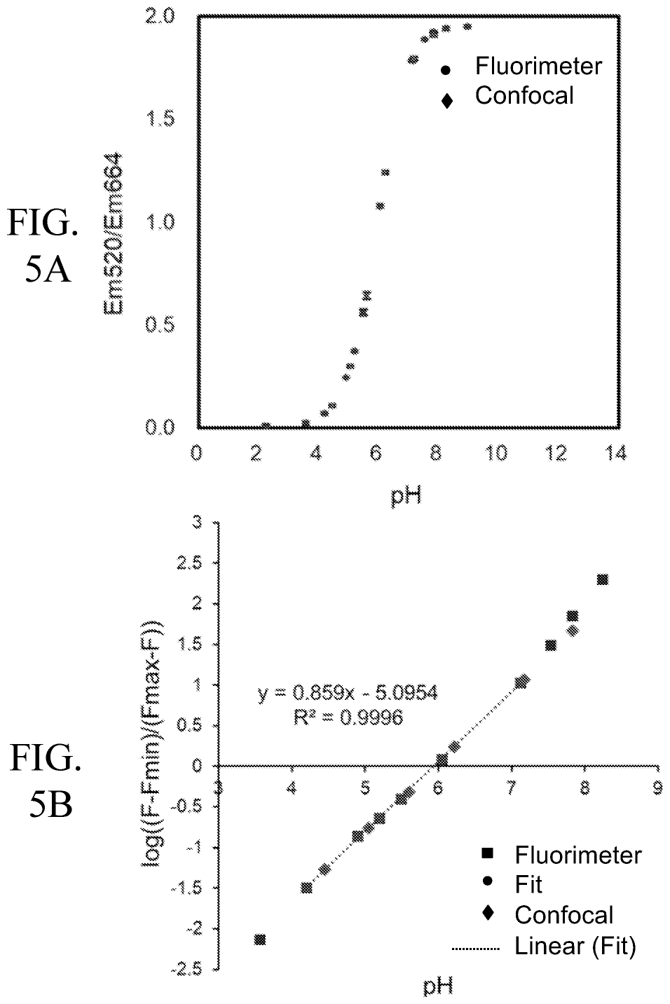

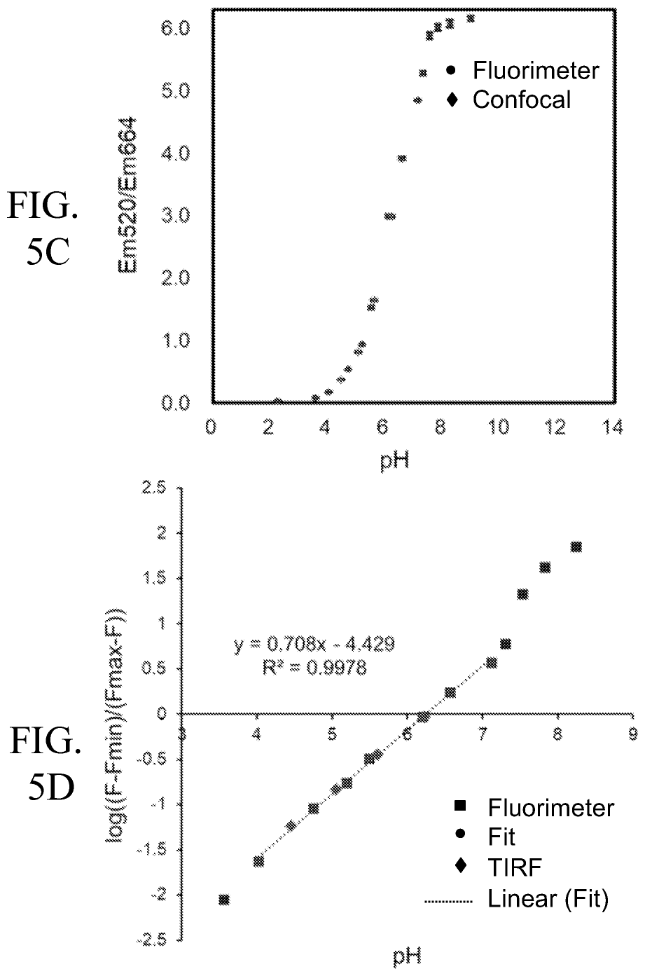

pH Nanosensor Calibration accounting for Heterogeneities in Sensor Particle Composition. Nanosensor calibration was first performed by measuring the fluorescence intensities of the nanosensors in standard solutions of known pH as described herein. To that end, FAM-ATTO647N aC′ dots and C′ dots were dispersed in PBS solutions of varying pH and the fluorescence spectra measured using 495 nm and 647 nm excitation wavelengths for FAM and ATTO647N, respectively (FIGS. 2G-2H). The order of reference and sensor dye excitation did not affect the readout, demonstrating that their spectral separation was sufficient to prevent substantial bleed-over effects. The performance of the pH nanosensors was quantified by taking the peak intensity ratios of sensor and reference dye emissions at 520 nm for FAM and 664 nm for ATTO647N as a function of pH (FIGS. 2I-2J). These calibration data were further analyzed using the Henderson-Hasselbalch equation (Equation 1). This analysis is based on determining the proportion of protonated species in a given sample. Both FAM-ATTO647N C′ dots and aC′ dots showed linear responses to changes in pH in the relevant pH ranges (FIGS. 5B, 5D, 5F), when their ratiometric data were plotted against pH using a modified Henderson-Hasselbalch equation (Equation 2). To that end, responses of the nanosensors were also measured well above (Fₘₐₓ) and well below (Fₘᵢₙ) their expected dynamic range around the pKₐ of the FAM sensor dye (6.4). From this analysis, the calculated pKₐ values for FAM-ATTO647N aC′ dots and FAM-ATTO647N C′ dots were 6.3 and 5.9 (standard error of regression was 0.06 in both cases), respectively. It could only be speculated about the reasons for the small observed difference in the measured nanosensor pKₐ values. For example, it may be associated with different degrees of backfolding of the sensor molecules toward the particle core surface as a function of their self-association, the latter being stronger in aC′ dots as compared to C′ dots.

$$pH = - log [ {H}^{+} ] = {pK}_{a} + log ( \frac{[ Base ]}{[ Acid ]} ) \qquad ( 1 )$$

$$pH = {pK}_{a} + log ( \frac{{F}_{i} - {F}_{m i n}}{{F}_{ma x} - {F}_{i}} ) \qquad ( 2 )$$

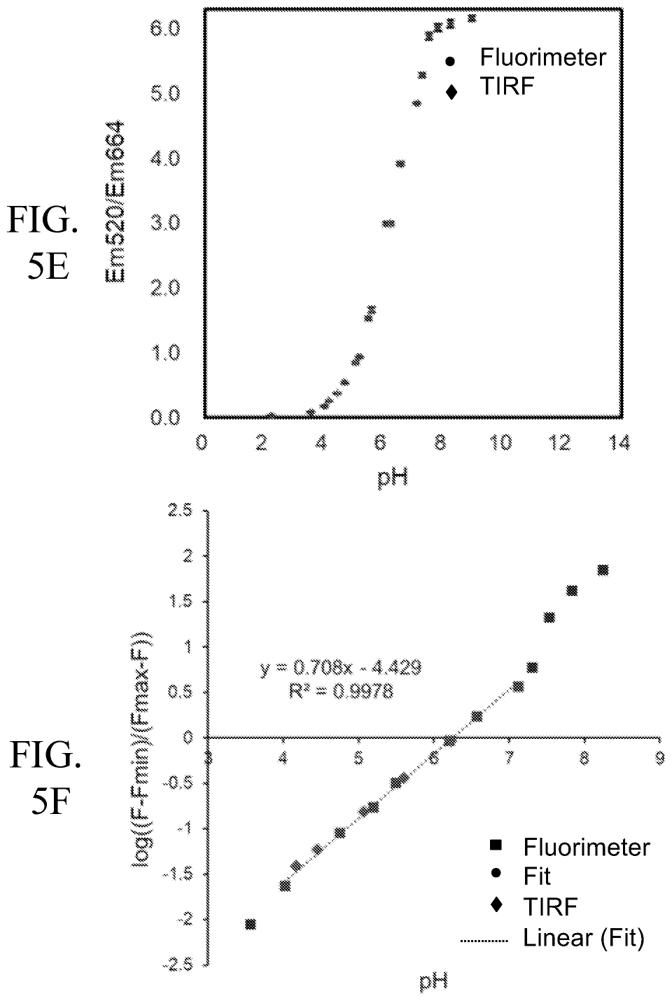

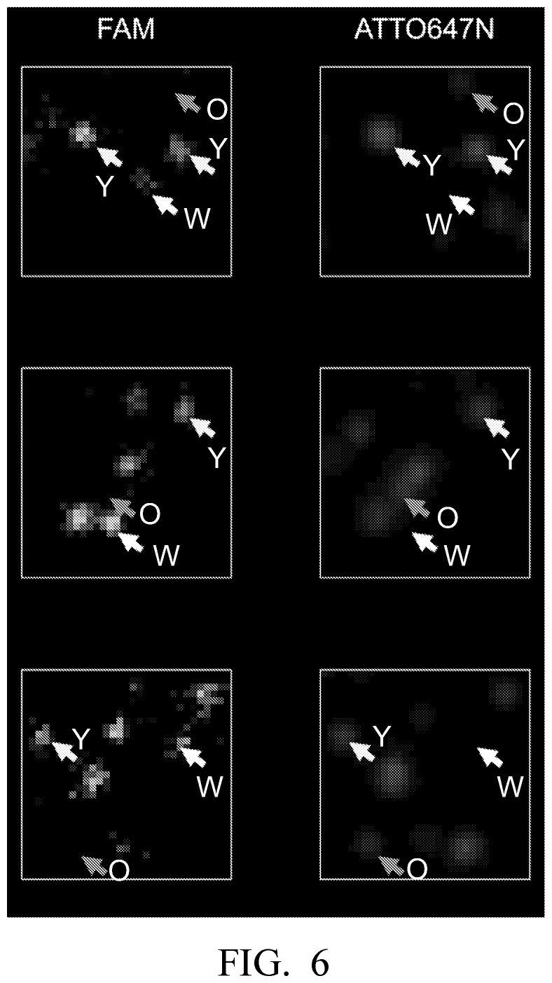

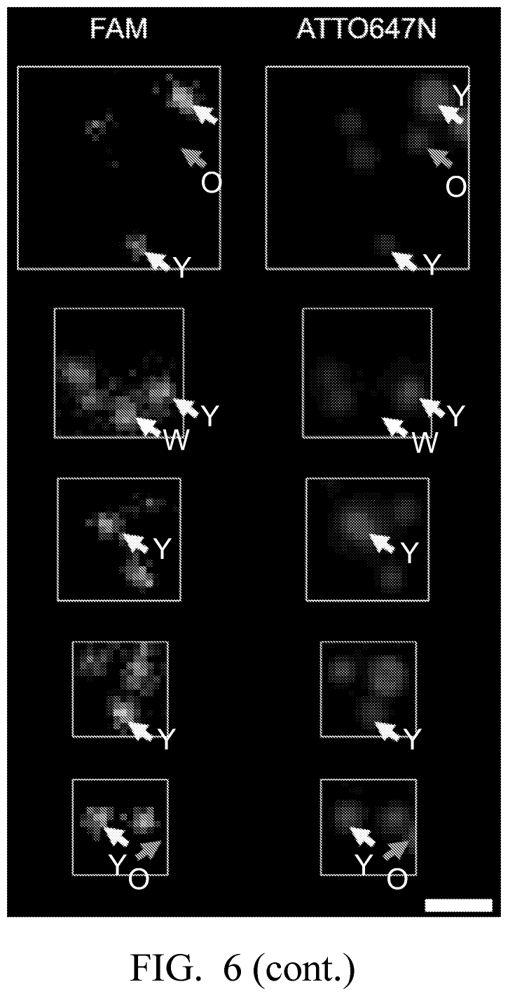

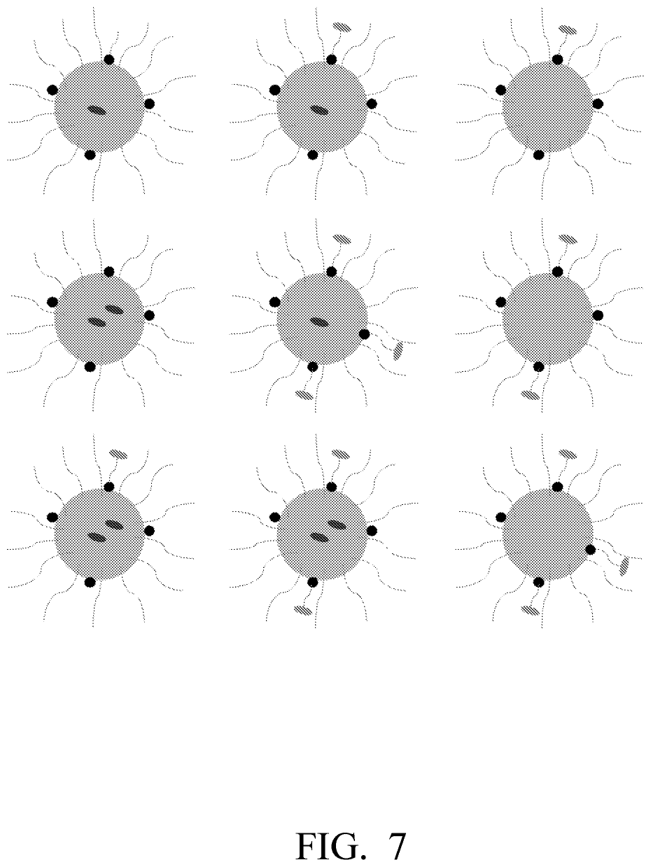

Based on the batch synthesis approach used here and as illustrated in FIG. 7, nanosensors of this study were expected to exhibit heterogeneities in their composition, including differences in the number of reference and sensor dyes per individual particle. In order to understand how this heterogeneity in FAM-ATTO647N aC′ dot dye composition influences results when moving toward STORM-enhanced ratiometric sensing, we performed fluorescence intensity measurements at a single particle level. To that end, diffraction-limited TTRF images were taken of FAM-ATTO647N aC′ dots immobilized on a glass dish (FIG. 6) at a dilute enough concentration to expect separation of single particles. In these presumed single particle images, a considerable number of regions were found which only showed green (FAM) signal spots. Since the dye ratio of ATTO647N to FAM for aC′ dots as derived from a combination of FCS and UV/VIS spectroscopy was 1:2.5, this was not unexpected. For further analysis, only regions were taken into account which showed both green (FAM) and red (ATTO647N) spots. Since the ratiometric ratio was defined as FAMIATTO647N intensities, presence of ATTO647N signal spots was required when selecting for regions with spots to generate ratiometric pH values from. A representative selection of such regions are shown in FIG. 6. They showed colocalization of FAM (green) and ATTO647N (red) signal spots, indicating that most of the nanosensors in these preselected regions have both dyes associated with the same particle. However, even in this preselected set or regions there are still individual spots (white arrows (W), FIG. 6) where FAM signals exist without corresponding ATTO647N signals, or vice versa (orange arrows (0), FIG. 6), suggesting that there are aC′ dots that only contain either the surface conjugated FAM dye or the core encapsulated ATTO647N dye. Furthermore, for spots where both signals are present (yellow arrows (Y), FIG. 6), the FAMIATTO647N intensity ratios considerably vary, despite the fact that all images were taken in the same buffer under the same pH condition. These single particle imaging results are therefore consistent with considerable compositional heterogeneities in the number of sensor and reference dyes per aC′ dot derived from a single aC′ dot synthesis batch, as illustrated in FIG. 7.

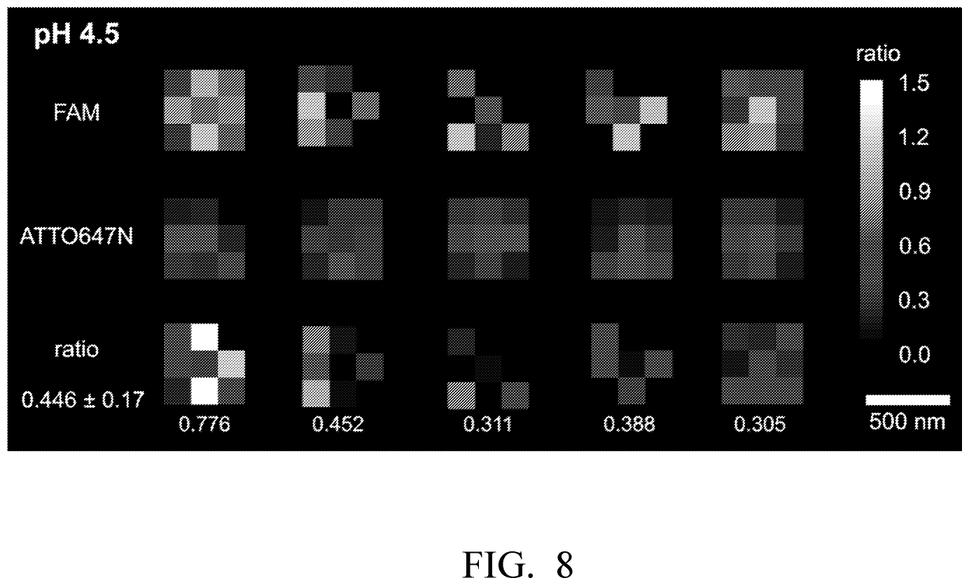

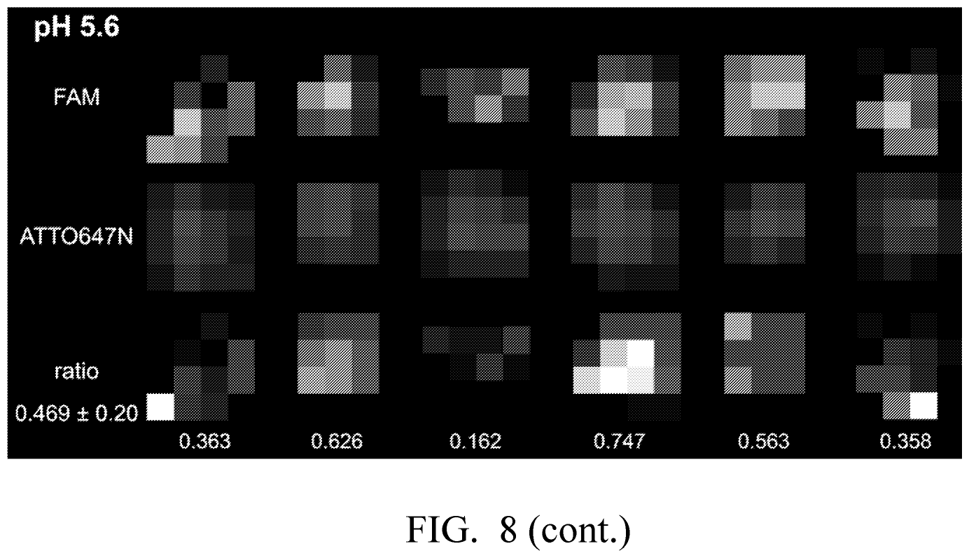

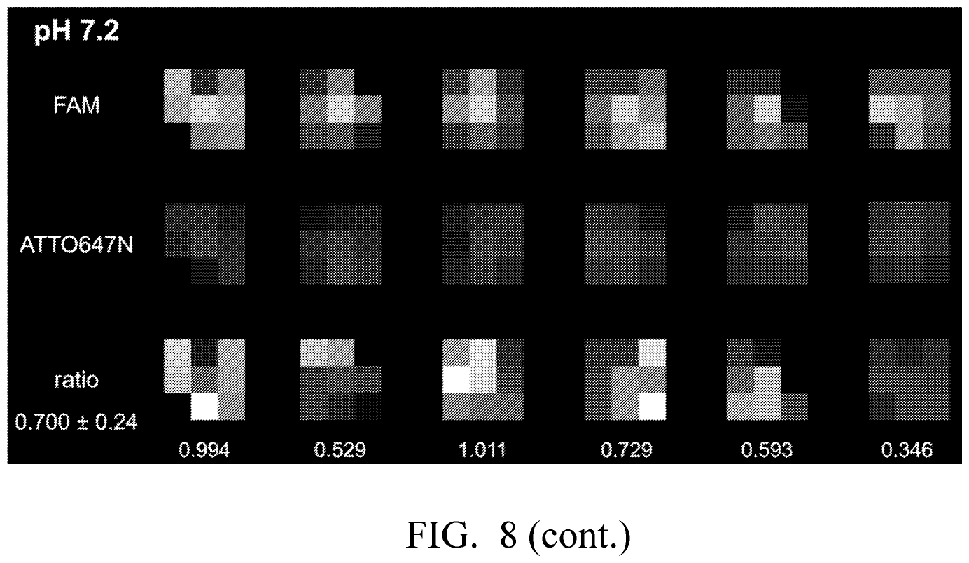

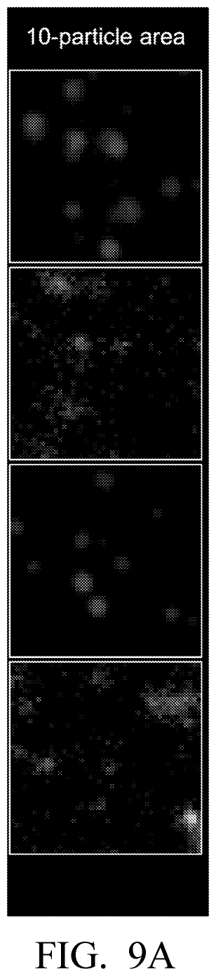

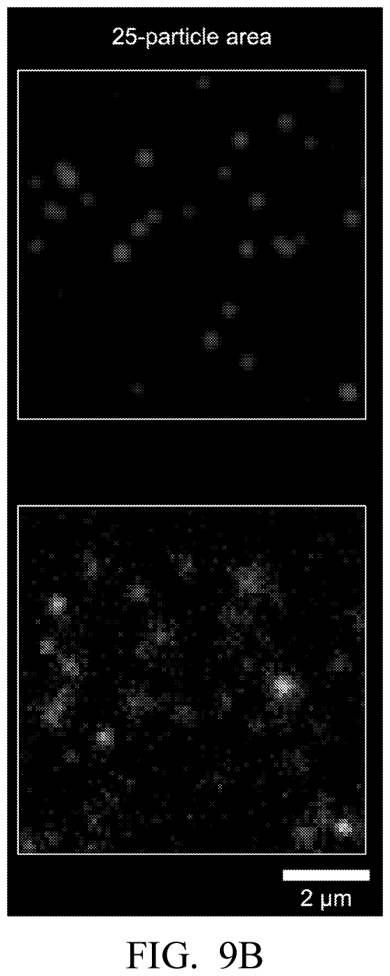

Quantitatively analyzing such single particle regions where both FAM and ATTO647N signal spots were present, and altering the pH of the imaging buffer the immobilized aC′ dots were immersed in (FIG. 8), it was found that the FAMIATTO647N intensity ratio still increased with pH, albeit with large deviations across different spots. Furthermore, the single particle derived ratios were generally smaller than what was expected from ensemble averaged calibration curves generated from solutions in spectrofluorometer measurements (FIG. 2K, FIG. 8). Due to the clustered nature of intracellular uptake of the ultrasmall aC′ dots, it was assumed that the probability of single particles being imaged within live cells for pH measurements is low. Instead, diffraction-limited fluorescence signals likely come from averages of multiple aC′ dots clustered within proximity of each other. Therefore, to quantitatively account for the observed heterogeneities in particle composition in super-resolution-enhanced live-cell ratiometric pH sensing, the number of particles was determined over which results needed to be averaged (FIGS. 9A-9B) before the FAMIATTO647N signal ratios approached their ensemble averages as measured in solution using the fluorometer. The “number of particles” in a select area of aC′ dots immobilized on a glass dish was determined by the number of isolated spots observed in the ATTO647N channel, not only because of the ratiometric signal definition, but because the number of particles within a confined space was quantified using STORM localizations generated from ATTO647N signals, as will be described below. Using this approach, from data displayed in FIGS. 2K-2M, it was found that the FAM to ATTO647N signal ratios approached the results of ensemble measurements as we increased the number of spots/particles averaged over. Signal ratios were substantially smaller than the corresponding ensemble averages when looking at individual particles (FIG. 2K). This deviation from the ensemble averages decreased moving to averages over 10 particles (FIG. 2L). By averaging over regions containing 25 particles (FIG. 2M), it was found that the ratios and their standard deviations were comparable to measurements of ensemble averages in solution. On a single particle level, the inherent compositional heterogeneities in the FAM-ATTO647N aC′ dot dye incorporation prevents accurate ratiometric pH sensing; however, if the imaged region contains 25+ particles clustered together, accurate determination of their pH environment is possible.

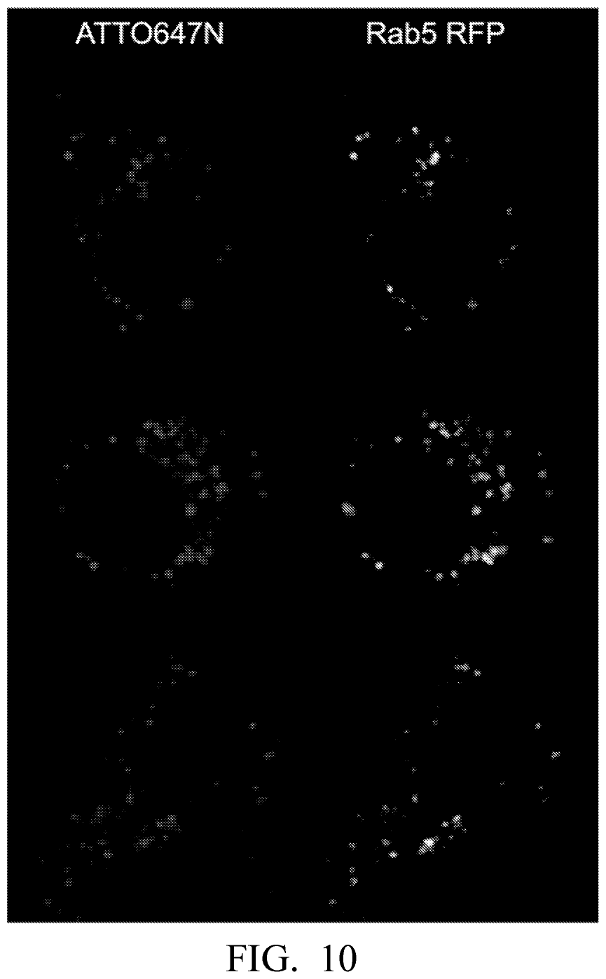

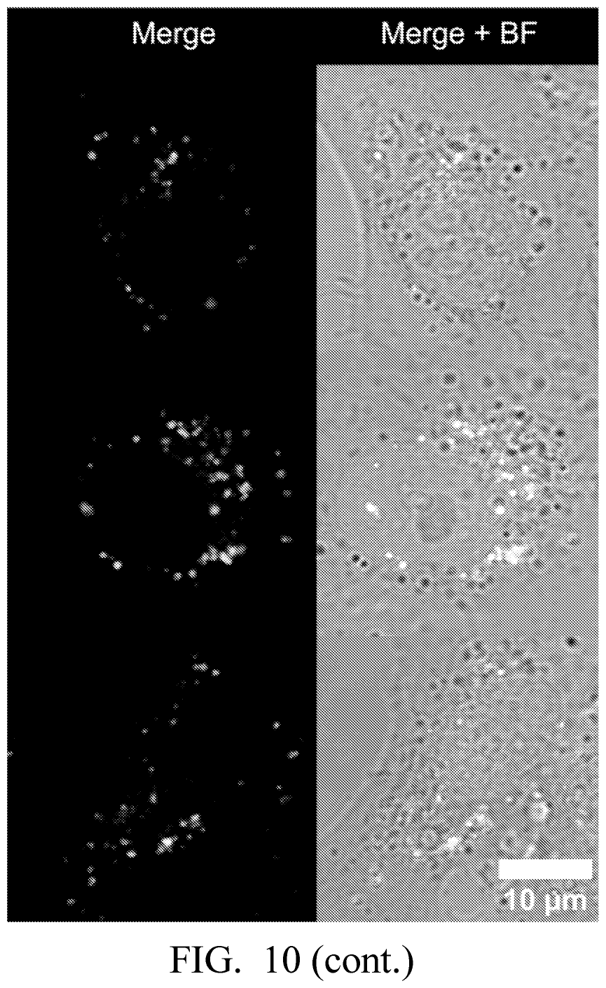

Diffraction-Limited Live-Cell Ratiometric pH Sensing. The live-cell imaging capabilities of these nanosensors were first tested in MDA-MB-231 TNBC cells. This cell line was used in previous studies describing the discovery of the ability of aC′ dots to blink under regular illumination enabling live-cell STORM imaging. Using the same cell line as a test bed for the first super-resolution-enhanced live-cell ratiometric sensing experiments therefore allowed direct comparison of results to these earlier studies. Imaging results of aC′ dots in MDA-MB-231 cells at the 60-minute time point had suggested that they are taken up by intracellular vesicles. To confirm this vesicular uptake mechanism with sensor-functionalized aC′ dots, MDA-MB-231 cells transfected with plasmids for RFP-fused-Rab5a, an early endosome marker, were serum starved for 24 h before incubation with FAM-ATTO647N aC′ dot nanosensors for 60 minutes. This approach allowed colocalization studies of Rab5a with fluorescence from the nanosensors. In live-cell confocal microscopy (FIG. 10), it was found that the ATTO647N signal from the nanosensors exhibited a high degree of, but not 100%, colocalization with the RFP signal from the Rab5a labeling. This indicated that FAM-ATTO647N nanosensors were indeed internalized into endosomes; however, the lack of total colocalization indicated that at the 60-minute incubation time point, while the majority of nanosensors is in early endosomes, some nanosensors may be associated with other stages of endocytosis. Next, TNBC cells were incubated with FAM-ATTO647N C′ dots/aC′ dots for various amounts of time: 2, 15, 30, 60, 120, 180, and 240 minutes (FIGS. 11A-11C), followed by washing away excess particles and fluorescence imaging using a confocal microscope as described herein. To that end, the TNBC cells were first starved for 24 hours in serum-free culture media, followed by incubation with C′ dot/aC′ dot sensors in full media in order to induce endocytic uptake of the nanosensors. The C′ dot/aC′ dot-labeled cells were then stained with Hoechst 33342 and imaged under a confocal microscope using three color channels (FIG. 11A): blue (Hoechst 33342 nuclear stain), green (FAM sensor dye), and red (ATTO647N reference dye). Taking the average fluorescence intensity ratios of the FAM emission (520 nm) to the ATTO647N emission (664 nm) across the entire cell, and using a calibration curve confirmed on this confocal microscope (FIGS. 2I-2J and FIGS. 5A-5D), the average cellular pH was determined at different particle incubation time points (FIGS. 11B-11C; as described herein). The first time point was taken at t=2 min of particle-cell incubation, which recorded a pH similar to that of the Hank's balanced salt solution (HBSS, pH ˜8) used as the imaging buffer. A monotonic decrease in pH as observed with incubation time, down to values below pH=5 at time points beyond 30 minutes, for both C′ dot and aC′ dot sensors.

The observed depression of pH as a function of incubation time is consistent with reported pH values of intracellular vesicles throughout different stages of the endocytic pathway. Combined with the observation that the C′ dot/aC′ dot sensors were visibly clustered within the cells (FIG. 11A), this suggested that C′ dot and aC′ dot ratiometric sensors both undergo endocytosis when incubated with serum-starved cells and end up in endosomal/lysosomal compartments, the latter known to exhibit pH values below 5. The similarity in the results further suggested that, as probed by cellular pH, transitioning from regular silica known from clinically-translated C′ dot core compositions to aluminosilicate cores did not lead to substantial differences in cellular processing.

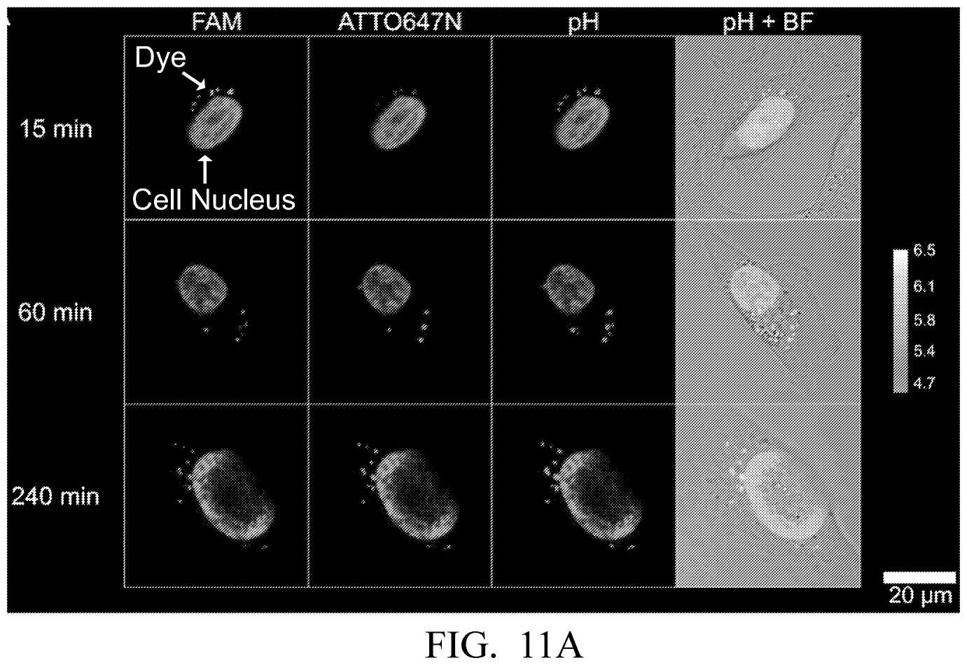

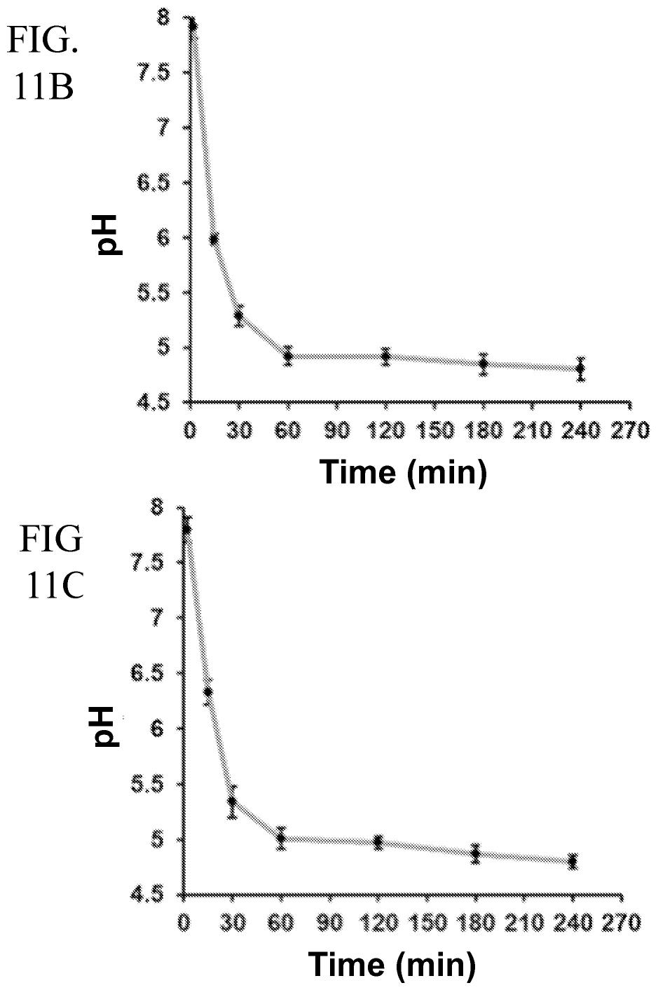

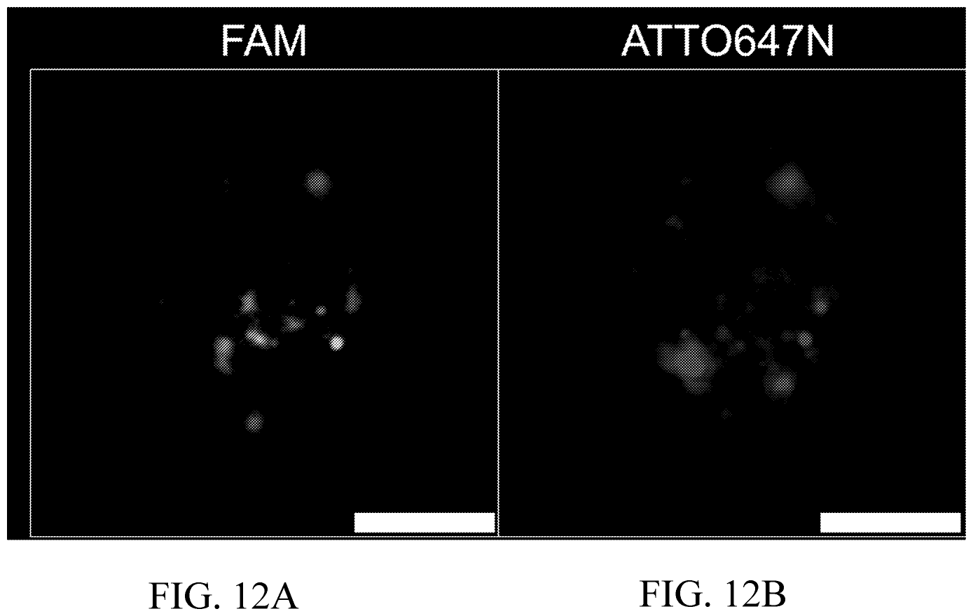

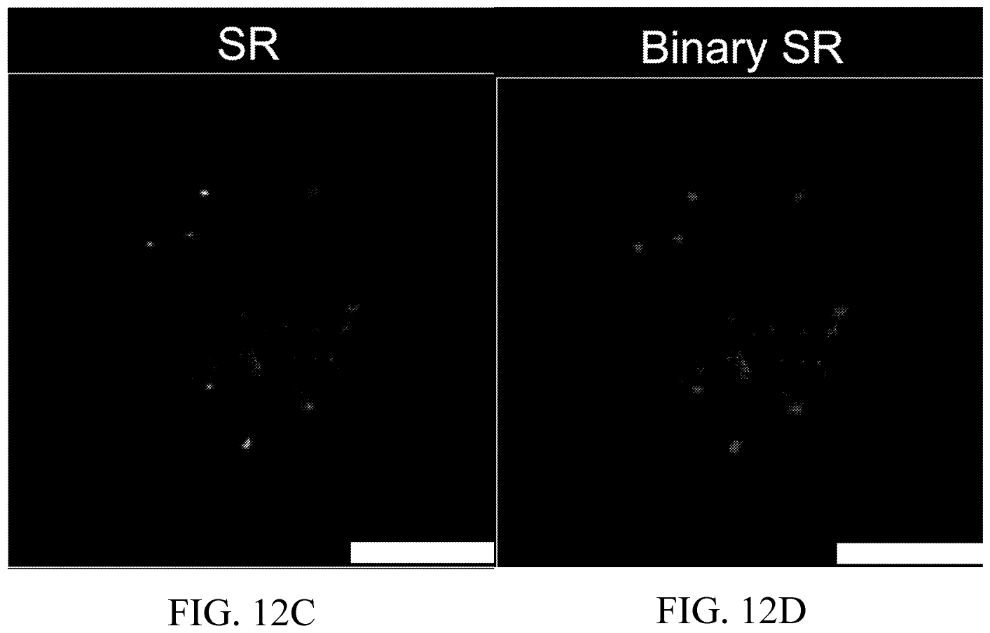

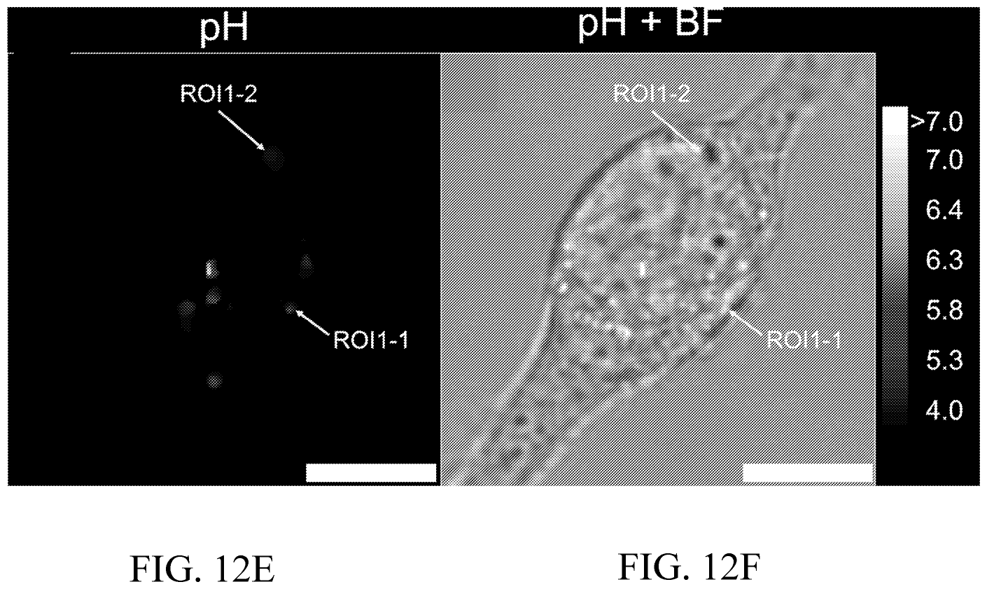

For STORM, fluorescence frames are typically acquired in the TTRF microscopy mode as a result of its superior signal-to-noise ratio compared to other fluorescence microscopy modes. In preparation of super-resolution-enhanced ratiometric pH sensing experiments and their analysis, we confirmed the calibration curve in FIG. 2I under regular diffraction-limited TIRF microscopy (i.e. not employing STORM), using FAM-ATTO647N aC′ dot solutions with known pH values (FIGS. 5E-5F). Next, diffraction-limited TTRF microscopy images were taken of MDA-MB-231 cells incubated with FAM-ATTO647N aC′ dots for 60 min (min=minutes(s)) (FIGS. 12A-12F). Evaluation of pH sensing was performed on averaged stacks of 50 frames collected in 50 ms intervals as opposed to a single frame collection. In order to be able to perform subsequent STORM experiments on the same samples in the red (ATTO647N reference dye) channel, low laser powers (0.5 mW) comparable to the foregoing work with confocal microscopy were used for 20 these TTRF-based experiments in red (ATTO647N reference dye) and green (FAM sensor dye) channels. This prevented rapid photobleaching, in particular of the more photosensitive FAM sensor dye (green channel). Isolated clusters of fluorescence intensities were again observed across the cells (compare FIGS. 12A-12B with FIG. 11A). Interestingly, likely due to wavelength-dependent penetration depth differences in TIRF microscopy, there were inconsistencies between the locations of clusters in green (FAM) and red (ATTO647N) channel-derived images in FIGS. 12A-12B. The 640 nm (red) excitation channel-based image revealed more objects when compared to the image derived from the 488 nm (green) excitation channel (in particular, see low intensity signals in red image in FIG. 12B). This is not surprising, however, given its ˜100 nm increased penetration depth (365 nm versus 278 nm). For further analyses, including construction of STORM imaging-based pH maps, regions that provided signal spots in both channels and covered similar areas were therefore focused on. Specific pH values for such regions could be determined (FIGS. 12A-12B, 12E) by mapping the pixel-based FAM and ATTO647N emission intensity ratios onto pH using the aC′ dot sensor calibration in TIRF mode (FIGS. 5E-5F). Despite the TIRF-based optical limitations for ratiometric sensing, the resulting pH values observed for the 60-minute incubation time point were consistent with the average cellular pH values as a function of incubation time shown in FIG. 11C.

Super-Resolution-Enhanced Live-Cell Ratiometric pH Sensing. For first proof-of-principle experiments on super-resolution-enhanced ratiometric pH sensing employing aC′ dot nanosensors, the focus of the optical STORM work of this study remained on the 60-minute incubation time point. A 60-minute incubation of MDA-MB-231 cells was also the time point investigated in a first aC′ dot-based live-cell STORM imaging efforts (excluding sensing actions) published earlier, thereby providing a useful testbed for quantitative comparisons of results. For STORM imaging, the laser power was increased in the red (640 nm) ATTO647N channel from 0.5 mW to 10 mW. Under these conditions reference dye ATTO647N covalently encapsulated in particles immobilized on glass showed low duty cycle optical blinking, as evidenced by the spikes in photon output significantly above the camera baseline level, appropriate for STORM and consistent with earlier results (FIG. 13A). In contrast, in the same immobilized particle experiments, but using high (10 mW) laser power in the green (488 nm) FAM channel, pH sensor dye FAM covalently attached to the particle ligand shell exhibited fluorescence in the first few seconds of collection, but did not exhibit the long-term blinking required for STORM (FIG. 13B). The photon outputs for single particles were calculated by integrating a 7×7 pixel area centered around the location of a single particle for each of 10,000 frames collected over 500 seconds. Per STORM experiment, 3000 image frames were taken over a period of 150 seconds in the red channel, which were then reconstructed using the ThunderSTORM plugin in ImageJ (as described herein). As expected, a visible increase in spatial resolution after the STORM reconstruction in the red channel (FIG. 12C) compared to diffraction-limited TTRF images (FIG. 12B) confirmed that FAM-ATTO647N aC′ dots were in fact conducive to live-cell STORM imaging.

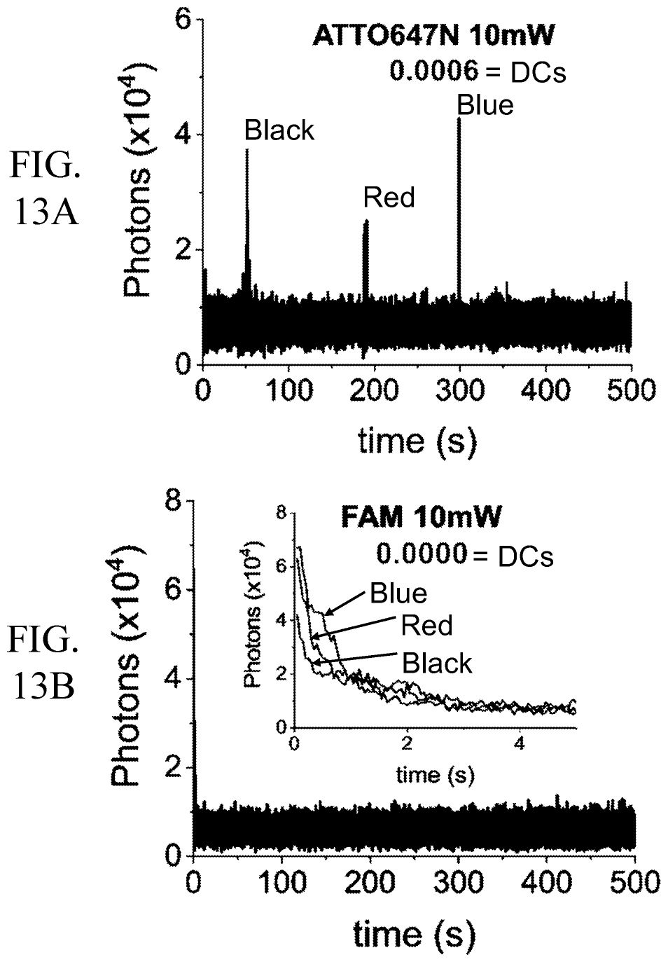

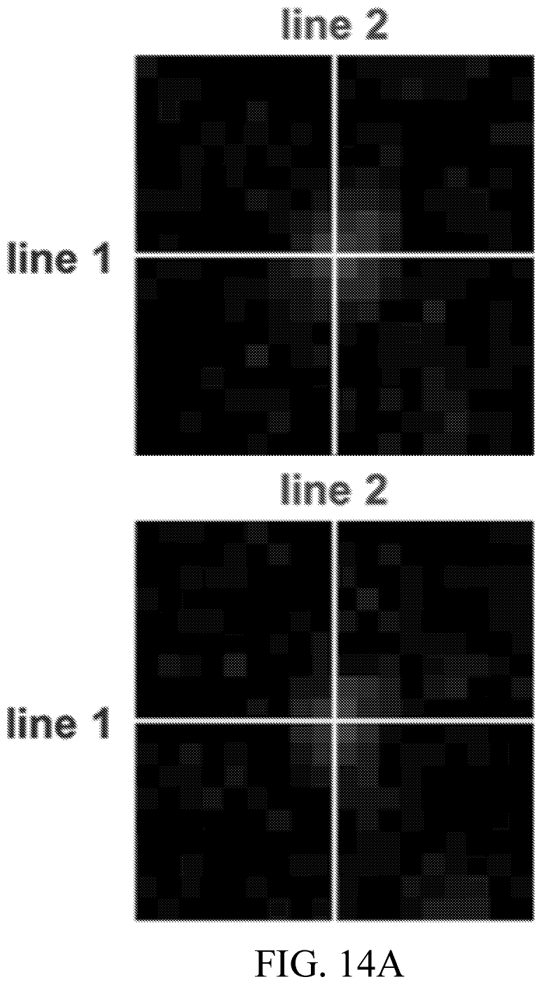

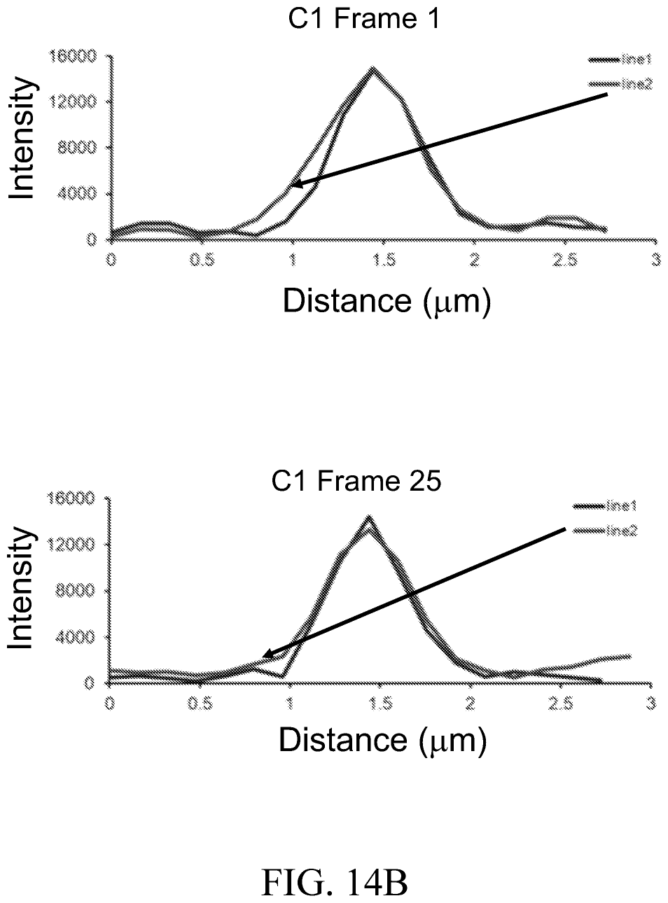

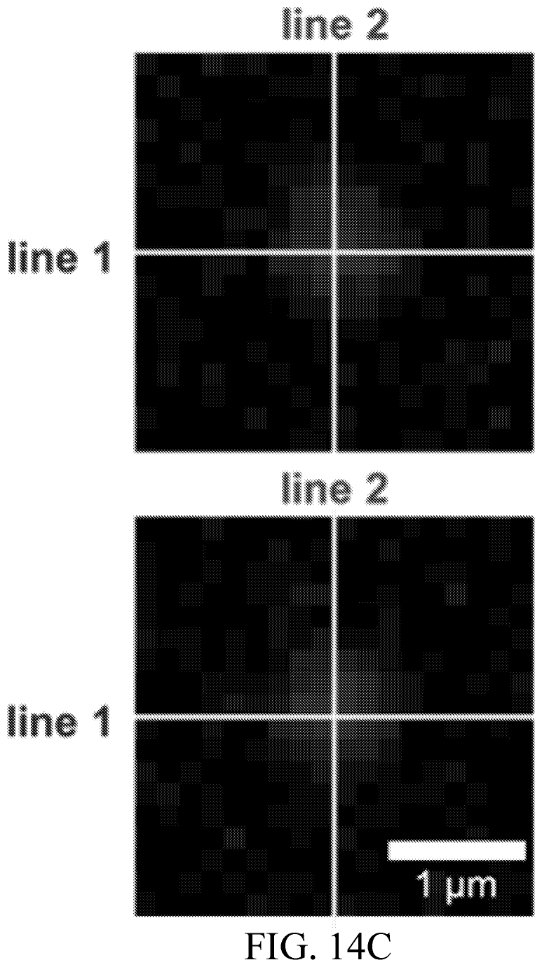

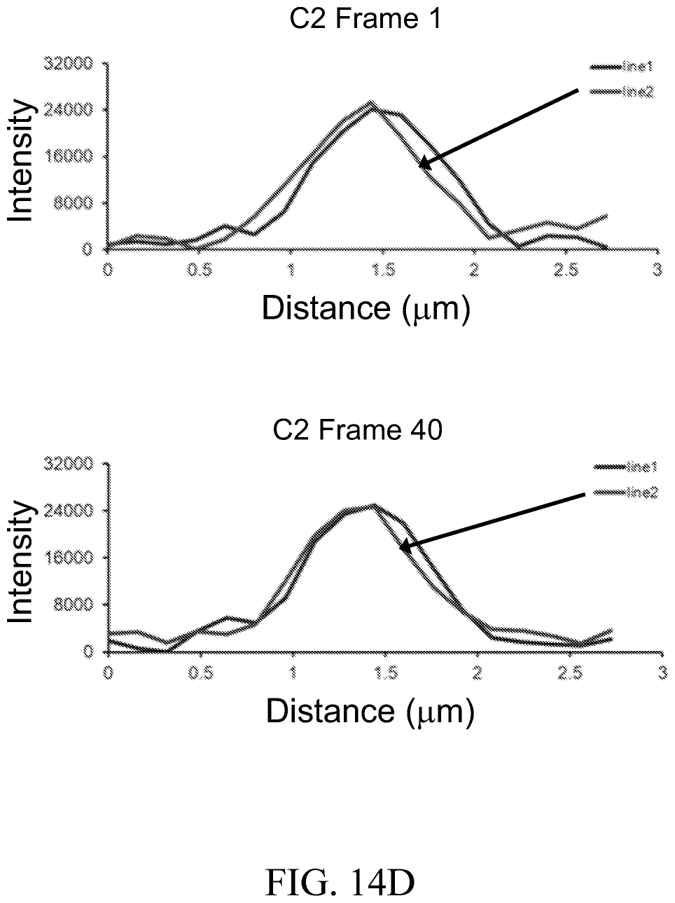

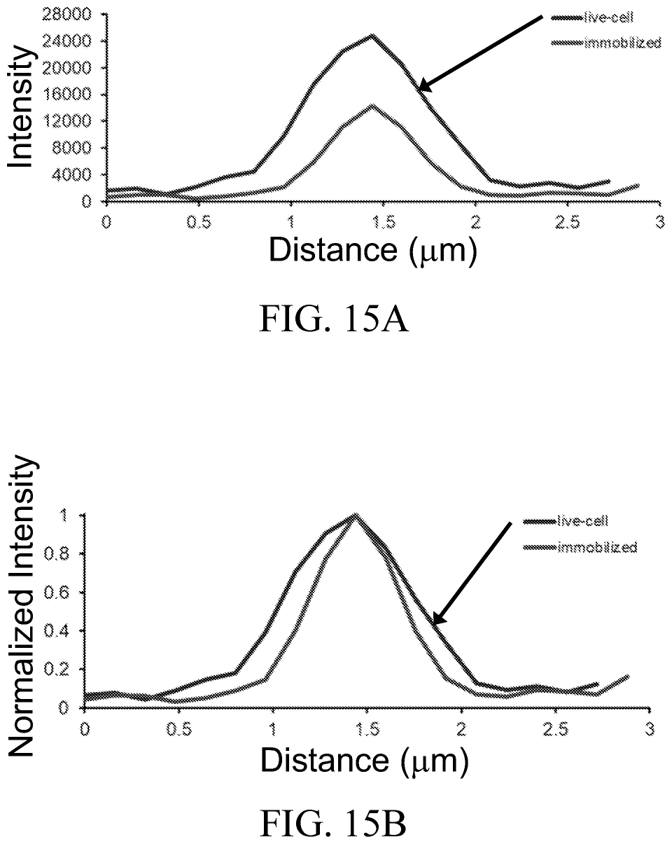

Despite the fact that aC′ dots in live cell imaging can freely diffuse while the 3000 frames are taken (collection time of 150 s), live-cell STORM reconstructions could still be performed, owing to the confinement of aC′ dot probes within intracellular vesicles. In fact, due to the freely diffusing sensors, STORM localizations can be used to map out the boundaries of the vesicle volume, as the aC′ dot probes can only diffuse within the confined spaces of the vesicles, spatially limiting blinks within these volumes. To assess the possibility of capturing specific diffusion paths during the integration time of a single frame (50 ms, during which ultrasmall aC′ dots can theoretically diffuse over the scale of microns when free in solution with properties similar to the endosomal environment, model calculation described herein) diffraction-limited PSFs obtained from immobilized single particle images of aC′ dots on a glass surface were compared to those obtained from live-cell images where aC′ dot diffusion is confined within vesicles. Intensity line profiles across isolated PSFs showed that the full width at half maximum (FWHM) is only slightly larger for live cell images as compared to images from immobilized single particles (FIGS. 14A-14D and FIGS. 15A-15B). Furthermore, intensity distributions of isolated PSFs remained fairly isotropic, and for STORM reconstructions could be well fit with Gaussian functions. These observations can be rationalized by the fact that the endosomal vesicles that contain aC′ dot sensors are below 500 nm in size, i.e. are substantially smaller than the spread of the PSFs, and are expected to have more or less spherical shape. Thus, despite probes exploring their local environment via diffusional processes during the 50 ms integration time of single frames, the STORM algorithm to extract sub-diffraction localizations of probes could still be applied.

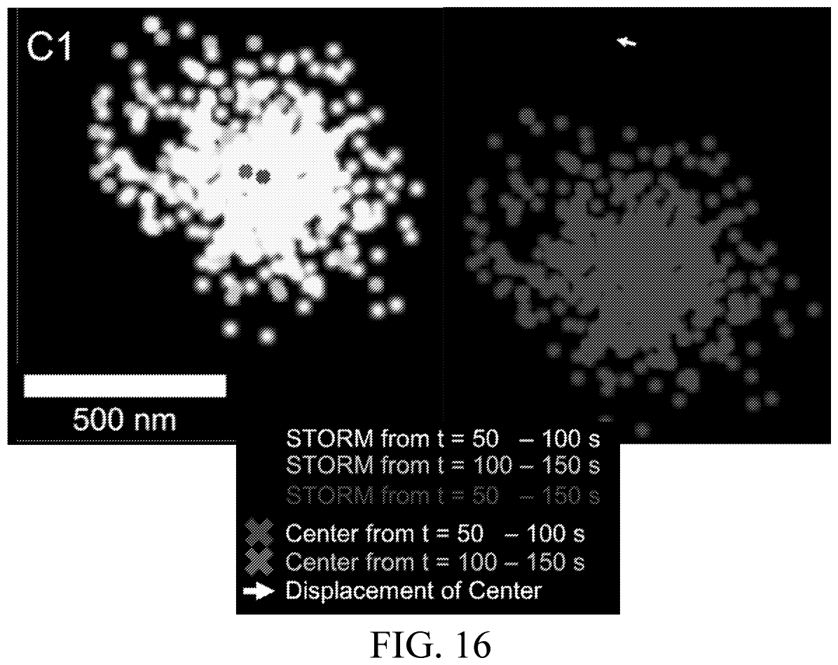

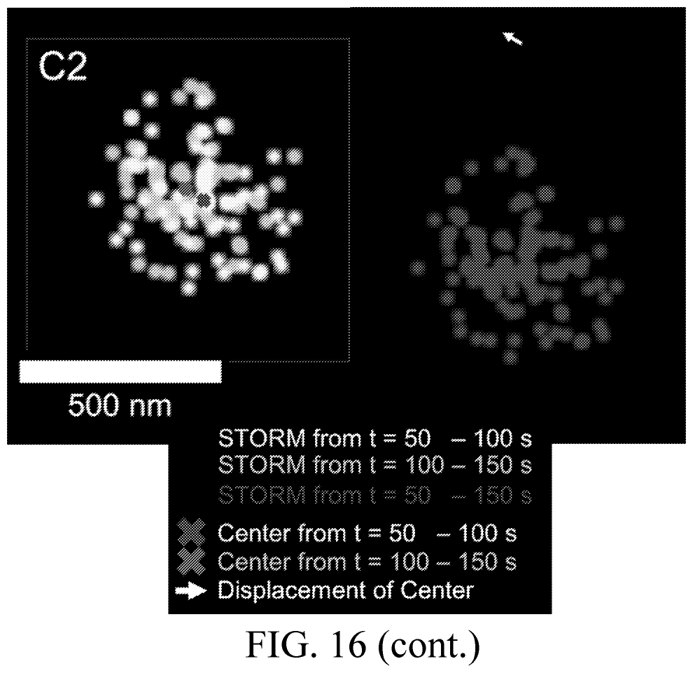

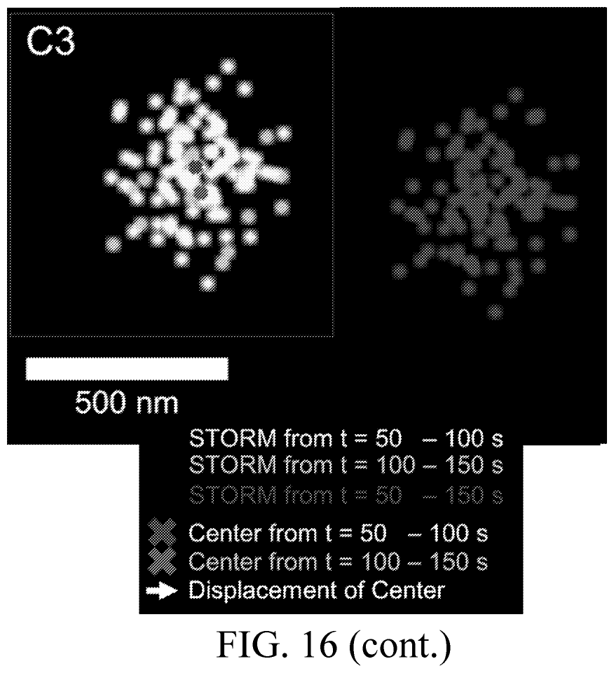

How STORM reconstructions from different frames evolve throughout the 150 s collection time was further analyzed (FIG. 16). To that end STORM reconstructions were performed using frames from 50 s to 100 s, from 100 s to 150 s, and from 50 s to 150 s. The centers of mass were then identified for the first two of these time windows (marked with crosses) to obtain a measure for the diffusion of the entire vesicles throughout a 100 s collection window. Three sample areas were chosen for this analysis as depicted in FIG. 16, from which it can be deduced that the earlier and later localizations occupy approximately the same areas, which can be interpreted as the volumes of the vesicles that the blinking aC′ dot probes are exploring via diffusion. By identifying the centers of mass of these localizations reconstructed from the two different time frames (i.e. 50-100 s, and 100-150 s) across the three sample areas, corresponding displacements (shown with arrows) of the clusters were calculated as well as their effective 2D diffusion coefficients, which were of order 10-20 nm²/s (shown in Table 1).

TABLE 1

| | C1 | C2 | C3 |
|---|---|---|---|
| Displacement in 1000 frames (nm) | 53.94 | 44.05 | 64.03 |
| x = √4Dt | | | |
| T = 50 s | | | |
| Effective 2D diffusion coefficient (nm²/s) | 14.55 | 9.7 | 20.5 |

Endosomal motility has been previously reported to be hindered by the cytoskeleton and consequently much lower than motility in the case of free diffusion in the cytosol, which is consistent with our observations and calculations. Furthermore, despite the 3 sample regions being chosen from the same cell image, the displacements throughout time had different directions and magnitudes, which implies that these effects were not caused by drift or cellular motion throughout the collection time frame. This slow vesicle diffusion makes it negligible relative to probe diffusion within vesicles, and therefore does not have any appreciable effects on the STORM analysis. However, as this analysis demonstrates, by taking advantage of the fact that aC′ dots are freely diffusing, one can not only map out the boundaries of the vesicular confinement, but can also track vesicular motion as a whole.

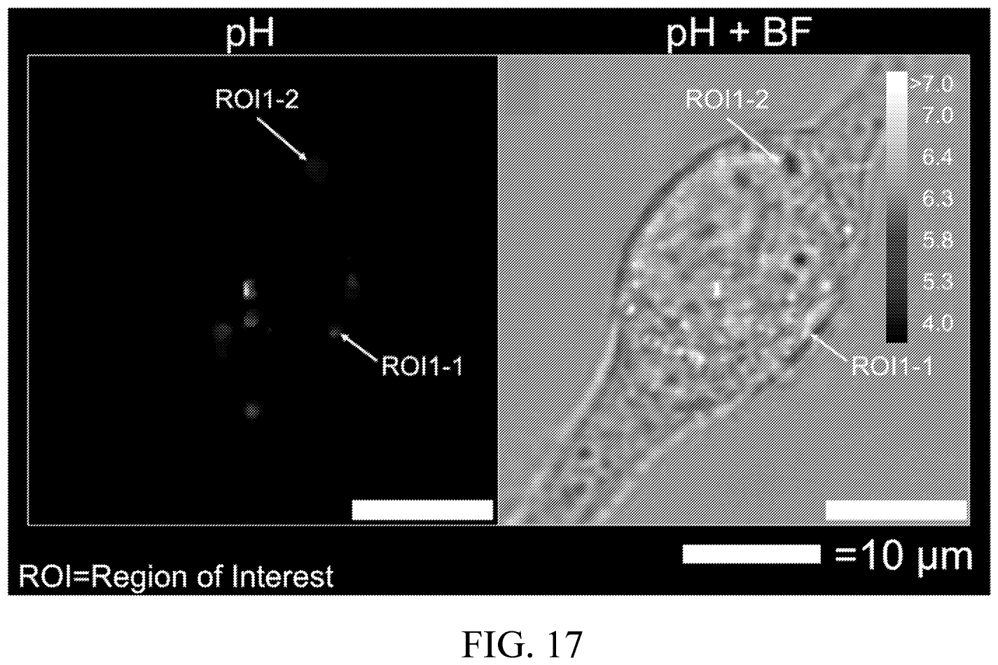

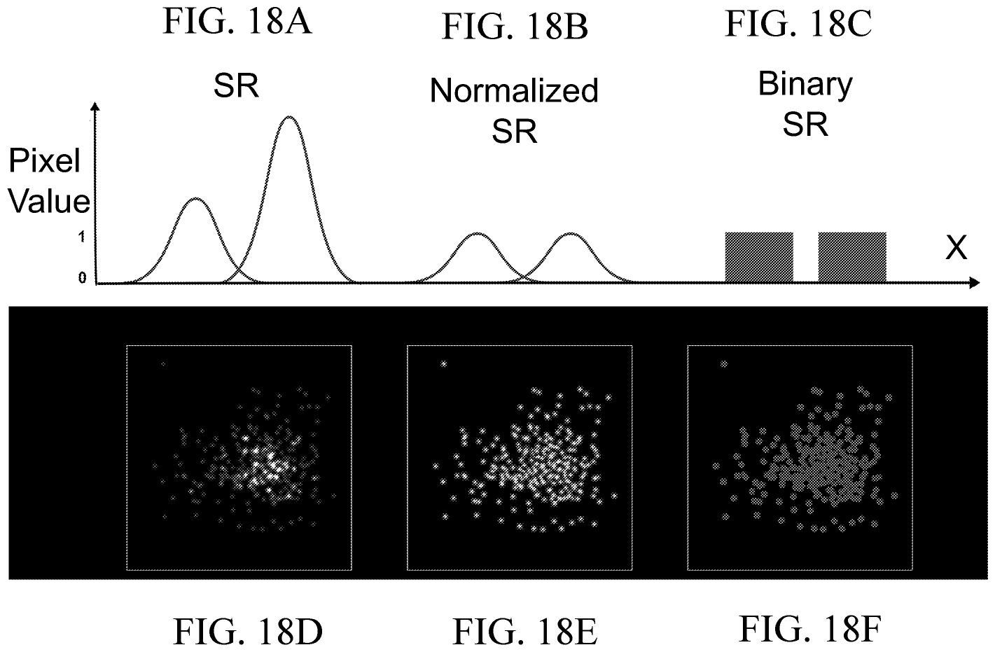

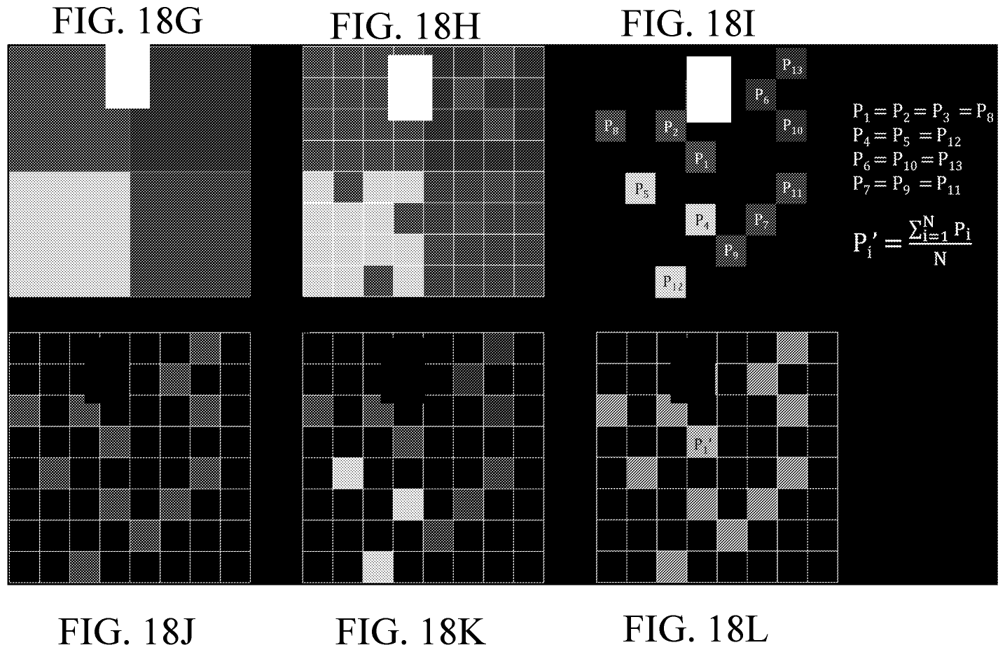

Construction of STORM-Enhanced pH Maps. Limited by commercially available fluorophores, a commonly used fluorescein-amidite derivative, FAM, was chosen as our pH sensor dye. Under the live-cell imaging conditions employed, this dye covalently linked to the aC′ dot ligand shell did not exhibit photoswitching behavior and therefore could not be used to generate STORM images in the sensing channel (FIG. 13B). However, enabled by the fact that ratiometric aC′ dot nanosensors can generate both diffraction-limited FAM sensor and ATTO647N reference signals in the green and red channels, respectively, as well as STORM-based ATTO647N reference signals derived from STORM analysis in the red channel, pixelated information from lower resolution TIRF microscopy sensing could be combined with that from STORM-based localizations in order to enhance the resolution of the imaging-based ratiometric pH sensor maps. In order to illustrate this approach, two regions of interest (ROIs), ROI1-1 and ROI1-2, were chosen from the diffraction-limited data set displayed in FIGS. 12E-12F (see FIG. 17 for higher-magnification images) to elaborate on the formation of STORM-enhanced pH maps. The final desired images were constructed using spatial coordinate information from STORM, essentially a binary localization mask, and pixelated pH values derived from pixelated intensity ratios obtained via regular TIRF microscopy and applying a pH calibration. To achieve this, the STORM localization information was simplified to only retain the spatial information, i.e. values of either 0 or 1 were assigned, where Is correspond to nanosensor presence and Os to nanosensor absence (FIGS. 18A-18F). This was achieved by first normalizing all point spread functions (PSFs) of the STORM analysis (FIGS. 18A, 18D) to a maximum peak height of 1 (FIGS. 18B, 18E), and then obtaining a near-circular shape by converting all values above a threshold value of 0.2 to 1, and those below to 0 (FIGS. 18C, 18F).

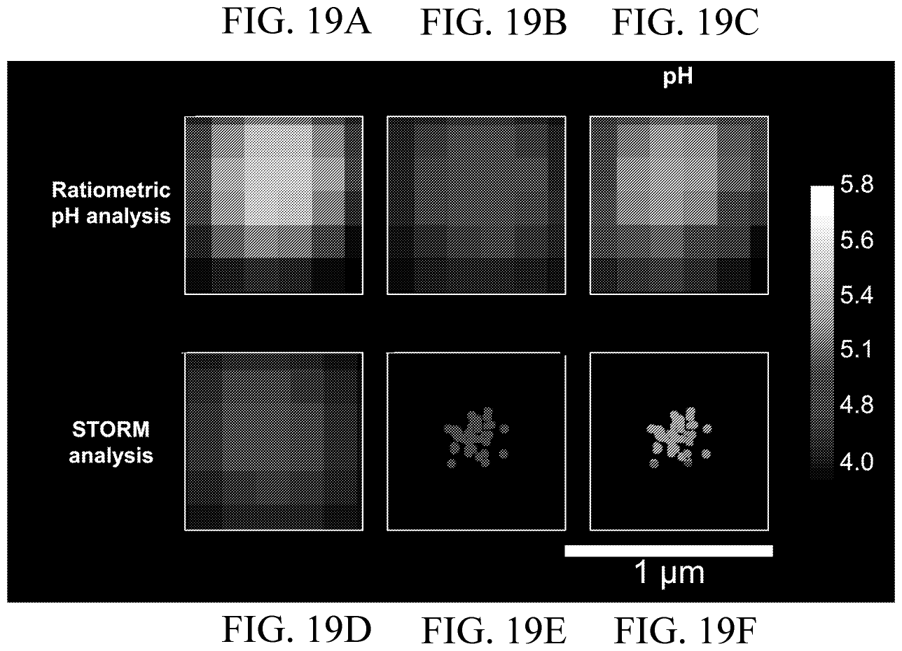

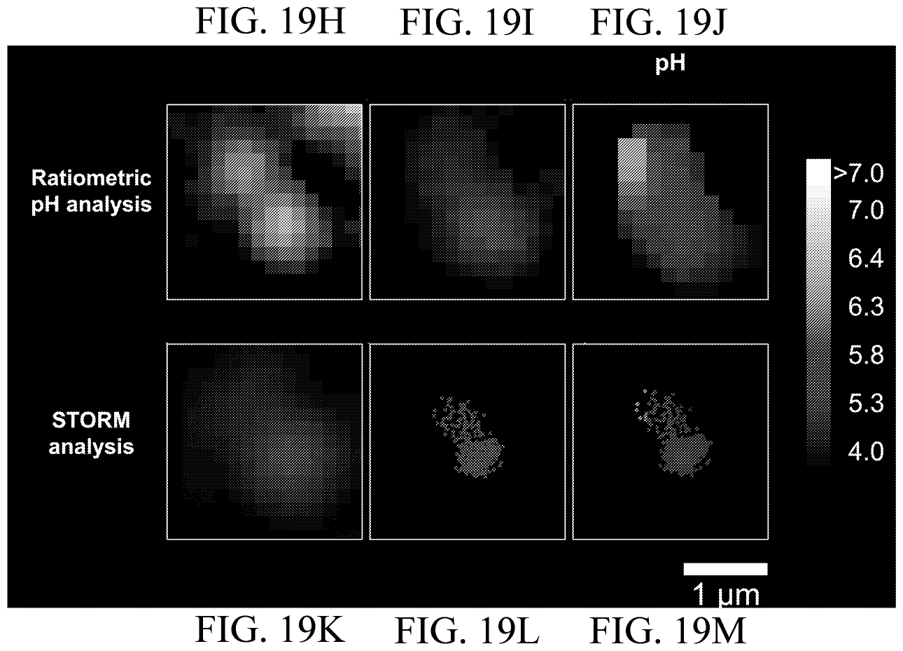

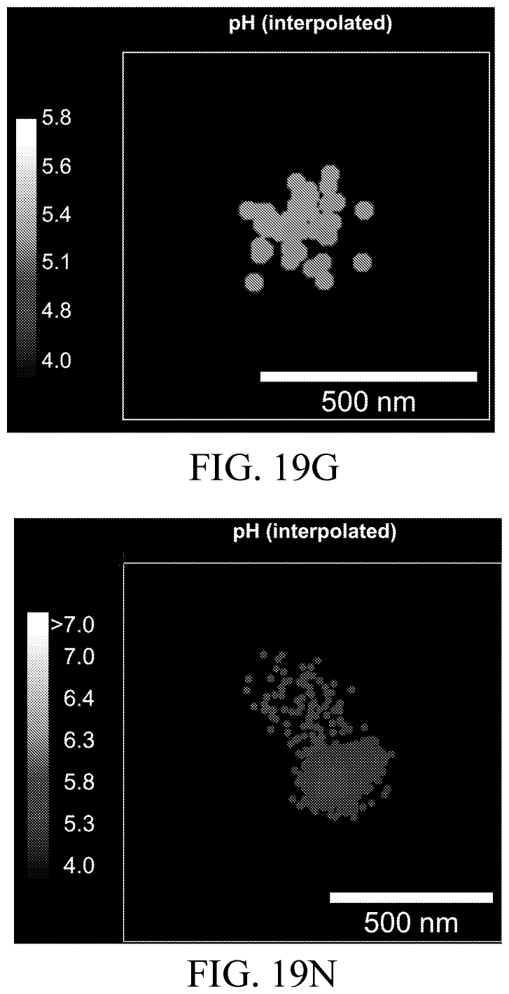

This binary localization mask was applied to the pixelated ratiometric pH-sensing values (FIGS. 18G-18L) via multiplication, selecting the intersection of the two images (FIGS. 18G-18H, 18J-18K). In doing so, localizations were assigned different pH values depending on which TIRF pH map pixel they were “resolved” from. As the number of pixels in the diffraction-limited TIRF image (˜6 pixels/μm) was increased to match that in the super-resolution STORM image (˜219 pixels/μm; compare grid sizes in FIGS. 18G, 18J; not drawn to scale), instead of directly dissecting one pixel into more pixels and assigning them the same value as the original pixel (FIGS. 18H, 18K), an interpolation method was developed that accounts for potential pixel intensity variations caused by the measured single particle heterogeneities. First, the central pixel (see FIG. 21 for illustration) associated with each emitter PSF area (FIG. 18J) is multiplied with the TIRF-based pH map in FIG. 18H, resulting in pixels containing information about super-resolution derived emitter location and diffraction-limited pH (FIG. 18K). Then, each emitter pixel-based pH value is replaced with a numerical average of its own value and that of its 24 nearest neighbors (NN), taking into account the earlier finding that for the specific aC′ dot sensor particles used here, averaging over 25 particles will approach accurate pH values consistent with solution measurements of ensemble averages. It should be noted that this NN-interpolation can be adjusted to whatever the degree of heterogeneity is as evidenced by single-particle analysis, as shown earlier. Larger degrees of heterogeneity will increase the number of NNs that need to be included in average value determination, while smaller degrees of heterogeneity will lower this number. In FIGS. 18G-18L, N in the summation formula represents the number of localized emitters averaged over to approach an accurate ensemble average, and i ranks distance from nearest to furthest (i=1 indicates the interpolated pixel, whereas 2<i<N indicates nearest localized emitter neighbor of the Nt^{h} ranking in distance). In the case where the number of localized emitters within an isolated cluster is smaller than N (25 in the case of FAM-ATTO647N aC′ dots described here), there likely will be effects from sensor compositional heterogeneities on the averaged pH values, and such clusters should not be considered. Panels in FIGS. 18I, 18L illustrate the case where the number of localized emitters that need to be averaged over is equal to N=13 (arbitrarily chosen). Step-by-step results of these procedures applied to ROI1-1 in FIG. 12E are shown in FIGS. 19A-19G. While the upper row in FIGS. 19A-19C displays the pixelated diffraction-limited TIRF microscopy images in green and red channels as well as the resulting pH map without interpolation, FIGS. 19D-19G display the corresponding STORM-enhanced results, with the NN-interpolated pH map shown in FIG. 19G. The substantial resolution enhancement in the pH maps moving from FIG. 19B to FIG. 19E and FIG. 19C to FIG. 19F and FIG. 19G is apparent from these comparisons.

Visually, this STORM-enhanced sensing scheme with NN-interpolation increased the homogeneity of pH values shown within the same isolated cluster area interpreted as a single vesicle (compare individual pixel values in FIGS. 19F-19G). Given the rate of proton diffusion relative to the timescales involved in the optical experiments (e.g. 50 ms integration time in STORM data acquisition), it is unlikely that the inside of a single vesicle exhibits substantial pH variations. Therefore, by taking the specific inherent single-particle heterogeneity of these sensor particles into account using the NN-interpolation, pH maps (FIG. 19G) were able to be generated that are more consistent with expectations of the pH distribution within a vesicle as compared to the results suggested by non-interpolated pH maps (FIG. 19F).

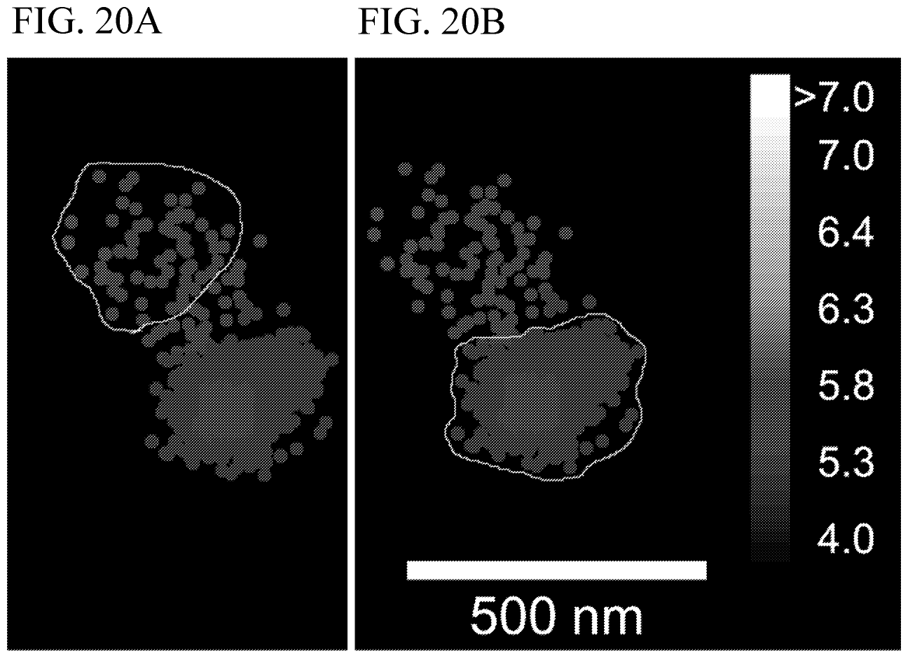

ROI2-1 shown in FIGS. 19H-19N (whole-cell data not shown) from a similar cell image as described in FIG. 12 contains two conjoined regions that even after NN-interpolation display clearly different pH values, i.e. around 5.8 in the upper left region (FIG. 20A) and around 5.5 in the lower right region (FIG. 20B). The pH map purely based on regular resolution TIRF microscopy (FIGS. 19H-19J) could be interpreted as a single domain. Closer inspection of the data set in FIGS. 19A-19N, in particular of the STORM-based localizations and associated pH maps (FIGS. 19K-19N) suggests, however, that these are two distinct regions, likely from two distinct vesicles that are in proximity to each other when projected onto the same imaging plane. In addition to differences in pH values, these two regions have visually different sensor probe packing density, with the upper region more loosely packed and the bottom more densely packed, corroborating the hypothesis of two separate vesicles. Having a super-resolution-enhanced ratiometric pH sensor instead of a diffraction-limited one therefore substantially increases the ability to spatially differentiate such structures. These observations motivate a closer look at how size and packing density of vesicles that have taken up aC′ dot particles correlate to pH.

Insights from Combined Quantitative Analyses of STORM-Based Live-Cell Imaging and Ratiometric pH Sensing. To establish such correlations, quantitative cluster analysis methods developed in a previous study were applied to the current STORM-based data sets obtained with ratiometric aC′ dot sensors. These methods allow assessment of vesicle diameter and estimation of the number of aC′ dots per vesicle. By attaching a pH sensor dye onto STORM-enabling aC′ dot probes, the ability to combine this type of information with information about the local pH environment around the nanoparticle probes has now effectively been gained.

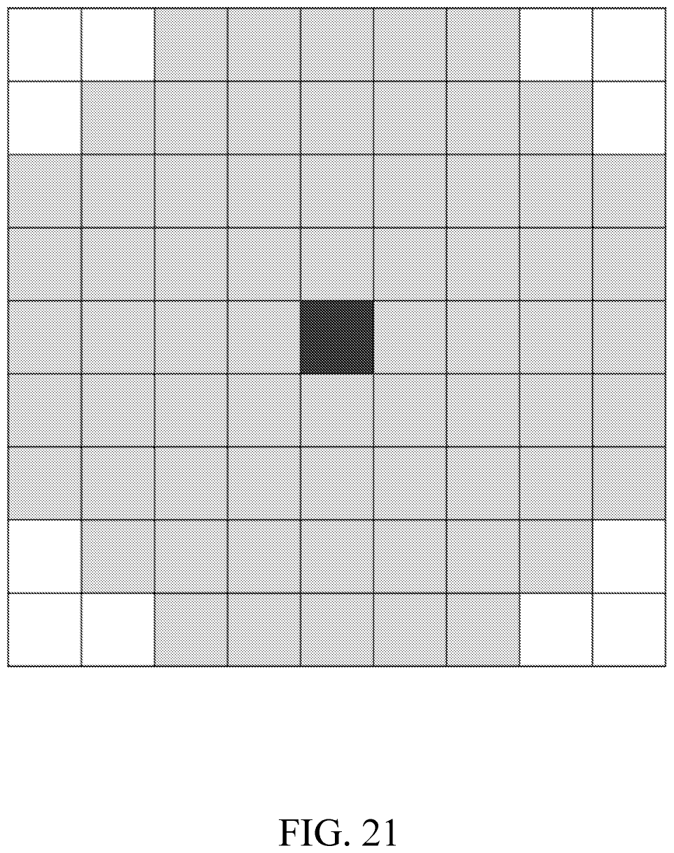

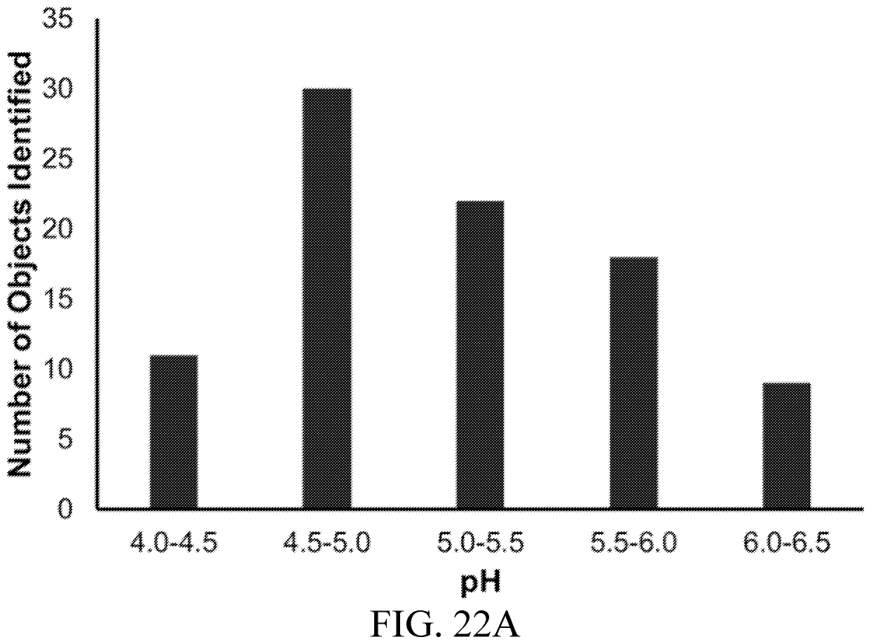

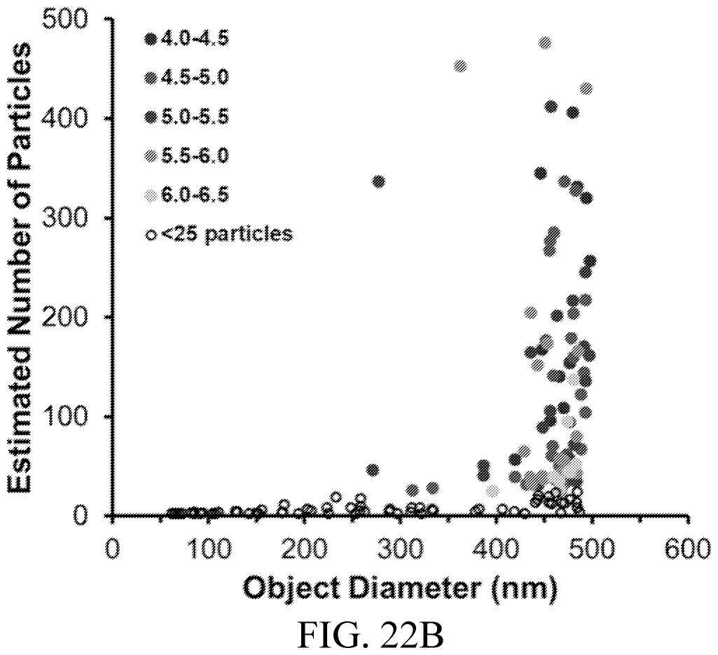

In this quantitative cluster analysis, the center pixel coordinates of each PSF (see FIG. 21) were used to localize the position of the nanosensors. As is apparent from binary STORM localization masks in FIGS. 19E, 19L and pointed out earlier, these localizations are typically clustered. In a previous study, this clustering was interpreted as the result of particles confined in cellular vesicles and a simple algorithm was developeded to estimate the size of these clusters and, therefore, the size of the vesicles from an average over the largest distances between any two localizations within such a cluster. Furthermore, from the number of STORM-based localizations per cluster/vesicle obtained from collecting image frames over a given period of time, and knowing the number of aC′ dot blinks per time from separate measurements, the number of particles in a specific vesicle can be estimated. In the current study, this information was combined with information about local pH. To that end, imaging results were analyzed from 11 different MDA-MB-231 cells incubated with FAM-ATTO647N aC′ dots for 60 minutes as described before. Applying a minimum threshold of 25 particle/vesicle filter, 90 local signal clusters interpreted as 90 cellular vesicles across these cells (165 vesicles before applying minimum threshold filter) were analyzed. Results of the combined quantitative imaging data analysis are displayed in FIGS. 22A-22B, including the number of vesicles identified in different pH ranges (FIG. 22A) and further associating the pH of each vesicle to a plot of the estimated number of particles per vesicle as a function of vesicle sizes (FIG. 22B). Please note that in these plots average vesicle pH values were obtained by averaging over all measured/interpolated pH values within a cluster/vesicle. Vesicles with fewer than 25 particles, i.e. below the threshold of N=25 localizations for which accurate pH values could be determined, are displayed as open circles.

12% (11 out of 90) of the analyzed vesicles fell in the 4.0-4.5 pH increment, 33% in the 4.5-5.0 (30 out of 90), 24% in the 5.0-5.5 (22 out of 90), 20% in the 5.5-6.0 (18 out of 90), and 10% in the 6.0-6.5 (9 out of 90) increment. Estimated vesicle diameters varied between 50 nm and 500 nm, with the estimated number of sensor particles per vesicle increasing with vesicle size. The maximum estimated number of particles per vesicle was near 500, which was a 2-fold increase from previous observations, while the size range from 50-500 nm was similar. This is consistent with the fact that the incubated particle concentration was doubled in the present work, while the endosomal pathway likely stayed similar. By comparing un-interpolated pH data to NN-interpolated data, it was found that the difference in average vesicle pH was minimal. As expected, this comparison confirms that single particle heterogeneity does not substantially affect average vesicle pH measurements, as long as there are enough particles within each vesicle in order to reflect ensemble averages. Having a super-resolution-enhanced nanosensor probe that simultaneously provides information on vesicle physical size, cargo quantity, mobility, and pH conditions is a substantial step toward better understanding the cellular processing of such nanoparticles. Analyses of a larger number of cells and vesicles over a longer incubation time period rather than the single 60-minute time point of the proof-of-principle experiments shown here are necessary, however, to fully accomplish this goal.

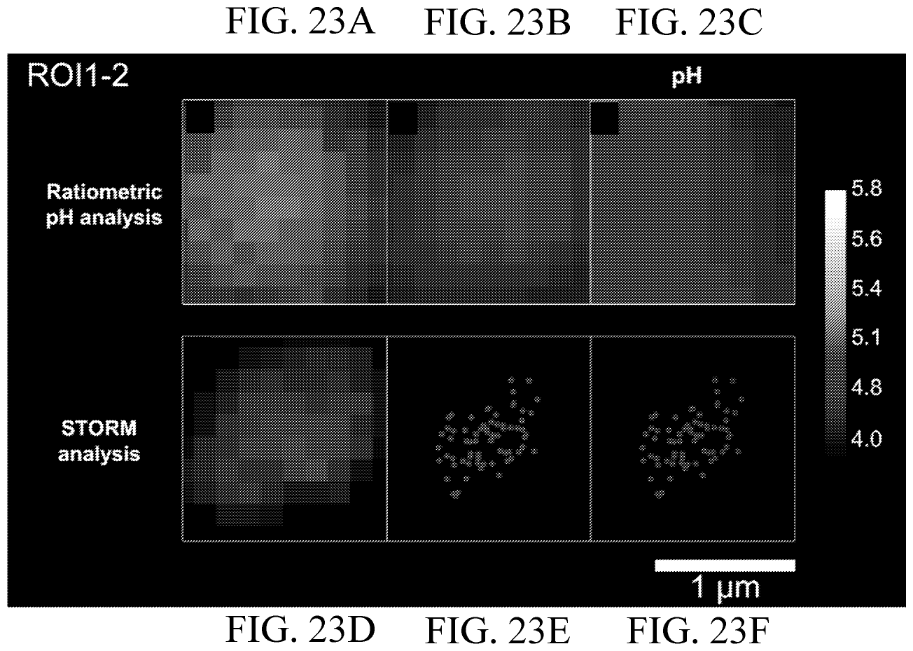

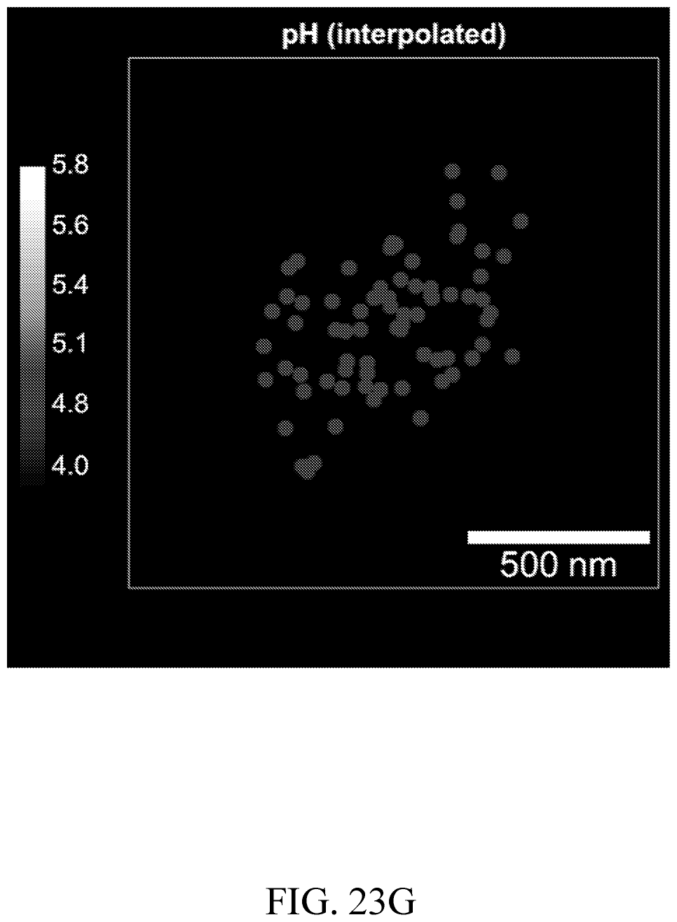

In addition to the 90 vesicles with sizes of 500 nm or smaller analyzed in FIGS. 22A-22B, 2 clusters of localizations were also observed with relatively homogenous pH<5 that had average diameters substantially larger than 500 nm. Results for one such cluster are shown in FIGS. 23A-23G. Their relatively homogeneous localization density and pH suggested that these are likely distinct structures, as opposed to 2 or more neighboring structures such as those shown in FIGS. 19H-19N. Compared to the smaller sized structures identified and analyzed in FIGS. 22A-22B, these clusters constituted a very minor population that might be associated with vesicles maturing into later stage endosomes.

In summary, an ultrasmall fluorescent core-shell aluminosilicate nanosensor (FAM-ATTO647N aC′ dot) was developed with commercially available reference (ATTO647N) and sensor (FAM) dyes covalently attached to particle core and shell, respectively, enabling live-cell super-resolution-enhanced ratiometric pH sensing via STORM. FAM-ATTO647N aC′ dot sensors as well as regular, non-blinking FAM-ATTO647N C′ dot sensors as controls were characterized by a combination of techniques including FCS, UV-Vis spectroscopy, TEM, and GPC demonstrating uniform ultrasmall particle sizes below 10 nm and low numbers of reference and sensor dyes per particle. Sensor calibration was accomplished in ensemble fluorometer measurements as well as in confocal fluorescence and TIRF microscopy setups, consistently demonstrating linear sensor responses within physiologically relevant pH ranges. In order to address expected heterogeneities in nanosensor composition across different particles from the same synthesis batch, single-particle imaging was used to determine the minimum threshold number of particles required in a local area to be averaged over in order to achieve pH measurement accuracy equivalent to ensemble measurements in solution. For the aC′ dot sensors synthesized in this study this number was 25. Comparison of aC′ dot and C′ dot ratiometric sensors in incubation-time-dependent confocal microscopy measurements of averaged cell pH revealed no substantial differences between the behavior of these two sensors in MDA-MB-231 TNBC cells. This suggested that the transition from silica to aluminosilicate in the sensor core does not substantially alter particle processing by these cells as revealed by pH. For proof-of-principle of super-resolution-enhanced ratiometric pH sensing, live-cell STORM and regular TTRF microscopy images were recorded of MDA-MB-231 TNBC cells incubated for 60 minutes with FAM-ATTO647N aC′ dots in the same field of view. Image processing methodology was developed that construct STORM-based super-resolution-enhanced pH maps of particles taken up by intracellular vesicles as suggested by particle clustering and pH. This was achieved by combining STORM-based spatial information with regular pixel-based ratiometric pH sensing values obtained from regular two-color channel TIRF microscopy in conjunction with a nearest-neighbor interpolation scheme in order to quantitatively account for particle heterogeneities in reference and sensor dye composition. It was demonstrated that having a super-resolution-enhanced ratiometric pH sensor, instead of only a diffraction-limited one, substantially increased our ability to spatially differentiate intracellular structures. Finally, this super-resolution-enhanced live-cell pH sensing analysis was combined with live-cell STORM-based quantification of vesicle size and number of aC′ dot probes per vesicle for a range of 11 different MDA-MB-231 cells and 90 individual particle clusters/vesicles (165 vesicles before applying minimum threshold filter). Results demonstrated that the overwhelming number of particle clusters could be assigned to vesicles with sizes below 500 nm, while only a small number of substantially larger vesicles was observed. Furthermore, the ability to estimate the number of particles confined to a single vesicle adds an important layer of self-validation in terms of the accuracy of pH measurements using particles with intrinsic compositional heterogeneities. The work presented here paves the way to the detailed mapping of the spatiotemporal evolution of nanoparticle processing by cells, of particular interest for cancer cells, via advanced optical techniques down to the nanometer scale. Experiments can simultaneously provide local information about the number of sensor probes localized to a particular structural element (here vesicles), the associated vesicle size, vesicular motion as a whole, as well as, via functional imaging, about metabolic parameters like pH or metal ion concentration (e.g. calcium) by developing the corresponding super-resolution-enhanced ratiometric sensors from commercially available sensor dyes. All particle syntheses, fluorescence microscopy and STORM experiments, as well as all data analyses described in this study were performed with relatively standard commercially available materials, fluorescence microscopy equipment, and analysis software. This work may therefore be useful to a large user community working in this area. Furthermore, since regular C′ dots are in advanced clinical trials and show promising potential for applications in cancer diagnostics and therapeutics, it is expected that this type of work will provide the foundation for developing an improved understanding of the intracellular mechanisms of such particle-based nanomedicines.

Experimental Section. Materials: Tetramethylorthosilicate (TMOS), (3-aminopropyl)trimethoxysilane (APTMS), aluminum-tri-see-butoxide (ASB), isopropanol, hydrochloric acid (HCl), ammonium hydroxide, 2M ammonia solution in ethanol and Atto647N maleimide (ATTO647N) were purchased from Millipore Sigma, 3-mercaptopropyltrimethoxysilane (MPTMS) and 2-[methoxy(polyethyleneoxy)6-9propyl]trimethoxysilane (PEG6-9-silane) were purchased from Gelest, DBCO-PEG4-NHS ester was purchased from Click Chemistry Tools, FAM azide 6-isomer (FAM) was purchased from Lumiprobe, and 5M sodium chloride (NaCl) solution was purchased from Santa Cruz Biotechnology.

Core dye conjugation: A day before particle synthesis, Atto647N maleimide was conjugated to 3-mercaptopropyltrimethoxysilane (MPTMS) in DMSO at a 1:23 molar ratio in a glove box under inert conditions. The conjugate mixture was left to react overnight.

C′ dot synthesis: A 10 mL water solution was tuned to basic pH by adding 20 L of 2M ammonia solution in ethanol. 68 μL of tetramethylorthosilicate (TMOS) and 0.25 mol of the ATTO647N-MPTMS conjugate from the day before were added dropwise to the reaction mixture while stirring (>600RPM) at room temperature. This ratio can be adjusted based on the desired number of dyes per C′ dot. After stirring overnight, 180 μmol of 2-[methoxy(polyethyleneoxy)6-9propyl]trimethoxysilane (PEG6-9-silane) was added dropwise. After stirring overnight, the reaction mixture was heated to 80° C. for 24 hours in a silicone oil bath with no stirring.

aC′ dot synthesis: 100 μL of ASB was diluted to 1 mL with isopropanol. Then, 78 μL of TMOS, 0.25 μmol of the conjugated ATTO647N-MPTMS, and 200 μL of the diluted ASB were rapidly added to 10 mL of 20 mM HCl while stirring (>600RPM) at room temperature. After 15 minutes of stirring, 150 μL of PEG6-9-silane was added dropwise. After 5 minutes of stirring, 280 μL of 14% ammonium hydroxide was added and the mixture further stirred for 12 hours. The reaction mixture was then heated at 80° C. for 12 hours in a silicone oil bath with no stirring.

FAM-DBCO-NH2_PEG-ATTO647N C′ dot/aC′ dot sensor syntheses: After the 80° C. heating steps in the C′ dot/aC′ dot syntheses, the C′ dot/aC′ dot solution was purified via GPC. 12 μmol of (3-aminopropyl)trimethoxysilane (APTMS) was added to the 10 mL reaction mixture and stirred overnight. 5 μmol of DBCO-PEG4-NHS ester was then added and stirred overnight. The C′ dot/aC′ dot solution was then purified via GPC (DBCO-NH2_PEG-ATTO647N C′ dot/aC′ dot), see below. Subsequently, the number of DBCOs per C′ dot/aC′ dot was determined from a combination of FCS and UV-Vis spectroscopy as described elsewhere. Based on this analysis, FAM dye was then added to the DBCO-NH2_PEG-ATTO647N C′ dot/aC′ dot solution at a 10:1 FAM to C′ dot/aC′ dot molar ratio. This amount may be adjusted based on the desired number of FAM molecules per C′ dot/aC′ dot.

Synthesis batch work-up and gel permeation chromatography (GPC): After sensor syntheses, the reaction mixture was syringe filtered through 0.22 m PVDF filters (Foxx Life Sciences) to remove large aggregates, and spun down twice in 30 kDa molecular weight cutoff spin filters (Corning) at 24,000×g, once for solvent exchange to water, and once for up-concentration. The GPC instrument used for product separation and analysis was equipped with a 275 nm UV absorbance detector (Bio-Rad BioLogic LP), and GPC was conducted with Sephacryl S-200 HR from GE Healthcare (now Cytiva) at a 2 mL/min flow rate in 0.155M NaCl solution. Desired particle products were separated from larger particle aggregates at shorter elution times and reaction educts at longer elution times relative to the product peak as discussed in previous publications. Eluted fractions (FWHM of the main particle elution peak) were collected by a BioFrac fraction collector (Bio-Rad). The resulting particle product was run through the GPC again for the final chromatograms shown in FIGS. 2A-2B. Fractions collected from the final run were subsequently solvent-exchanged to water from the GPC run solution via centrifugation in 30 kDa molecular weight cutoff spin filters (Corning) at 24,000×g for 3 exchanges.

Fluorescence correlation spectroscopy (FCS): Fluorescence correlation spectroscopy was conducted using a homebuilt laser setup, with a 633 nm solid state laser for excitation of core dyes of the FAM-ATTO647N C′ dots/aC′ dots. C′ dots/aC′ dots were suspended in DI water on a 1.5 coverslip glass imaging dish (MatTek) and placed on a water-immersion objective (Zeiss Plan-Neofluar 63x NA 1.2). Analysis of autocorrelation curves was performed as discussed in previous publications (see also FIGS. 5A-5F).

UV-Vis spectroscopy: UV-Vis spectroscopy was conducted on a Varian Cary 5000 UV-vis-NIR spectrophotometer. The samples were suspended in DI water or PBS buffer (pH=7.8).

Relative brightness assessments: The relative brightness of dyes in nanosensor aC′ dots compared to free dyes in solution were determined by first measuring the absorbance and emission maxima of nanosensor dyes and free dyes at similar concentrations in PBS buffer at various pH values. These solutions were absorbance-matched and the ratio of particle peak fluorescence and that of the unfunctionalized dye was obtained to calculate the relative brightness (see FIGS. 12A-12F). Each measurement was repeated 3 times and standard deviations were derived from the relative brightness determined from each set of measurements.

Nanosensor calibration: Calibration of the nanosensor performance was conducted using a Photon Technologies International Quantamaster spectrofluorometer. pH nanosensors were calibrated using PBS buffer (Gibco) titrated to known pH levels by hydrochloric acid and ammonium hydroxide. All fluorescence intensity results were averages and standard deviations of 3 measurements. Similar calibrations were performed in confocal and TIRF microscopies. MDA-MB-231 cells were incubated with nanosensors for 2 minutes and subsequently washed and imaged in buffers of known pH. Visibly isolated ROIs were chosen across 3 cells for each pH condition, each cell with 3 ROIs, and the resulting calibrations were generated using averages and standard deviations of the mean pixel values of these ROIs as described later in the Image Processing section

Particle immobilization for imaging: Nanosensor particles can be immobilized on glass slides either by inserting biotin into the PEG layer of the nanosensors and binding with streptavidin-coated glass slides as has been used in previous studies, or by directly casting nanosensor solution onto glass slides, allowing them to simply adhere to the glass substrate. No appreciable difference in blinking behavior was observed between these methods (data not shown). However, using biotin-functionalization for immobilization introduces a further layer of heterogeneity, as (similar to the addition of FAM to the particle surface) there is expected to be an uneven distribution of biotin across the nanosensors. Furthermore, we observed that particles only containing core dye, but no surface sensor dye, were more preferentially immobilized via the biotin route (data not shown). Therefore, to avoid further convolution of effects beyond the already complex heterogeneities in the nanosensor composition, we chose to directly cast nanosensors onto glass slides for any of the immobilized single-particle imaging experiments discussed in this paper.

Blinking statistics acquisition: Blinking statistics were acquired by exposing nanoparticle-immobilized glass slides to 10 mW laser power. Data collection was performed for a total of 500 s with 50 ms integration time under the TIRF microscope set up. Duty cycles were calculated using previously published methods. Single-particle fluorescence time traces for ˜100 individual particles were generated using a custom Matlab code described previously Duty cycle was calculated using a 100 second sliding window within the 200-400 second time range as described previously.

Single particle image acquisition for particle pH heterogeneity analysis: Particle immobilization and image acquisition was identical to that described in the previous section. However, the laser power for both the 488 nm and 640 nm laser lines was 0.5 mW and the image acquisition was only 50 total frames (2.5 total seconds). The pH was changed by imaging immobilized particles in the PBS solutions described in the nanosensor calibration section.

Cell culturing and fluorescence microscopy: MDA-MB-231 cells (ATCC) were cultured in complete media (phenol red free RPMI 1640 (Gibco) supplemented with 10% FBS (Coming) and 10,000/10,000 penicillin/streptomycin (Lonza)) at 37° C., 5% CO₂, and 90% humidity. 1×10⁵ cells were plated in a 1.5 tissue culture treated coverslip polymer dish (Ibidi) overnight followed by 24 hour starvation by replacing the complete media with incomplete media (phenol red-free RPMI 1640 with 0% FBS and 10,000/10,000 penicillin/streptomycin). Incomplete media was replaced with complete media containing 0.5 M nanosensors for 2, 15, 30, 60, 120, 180, and 240 minutes before triplicate washing in Hank's Buffered Salt Solution (HBSS; Gibco). The nucleus was labelled with Hoechst 33342 (Invitrogen) prior to imaging. Confocal microscopy was performed on a Zeiss 710 LSM Confocal Microscope with a 63x/1.4 NA Plan-Apo Objective. TIRF microscopy was performed on an Olympus IX81 microscope with a 100×/1.49 NA TIRF objective. Hoechst 33342, FAM, and ATTO647N were excited by 405, 488, and 640 nm diode lasers (Opto Engine LLC), respectively, at 0.5 mW for all laser lines. An Andor iXon 897 Life EMCCD was used at 50 ms integration times for all TIRF and STORM images with 50 total frames (2.5 total seconds) being collected for the former and 3,000 total frames (150 total seconds) being collected for the latter. 640 nm laser was used at 10 mW for STORM image collection.

Rab5a transfection and colocalization imaging: MDA-MB-231 cells were transfected with CellLight Early Endosomes-RFP (Invitrogen) overnight followed by were serum starvation for 24 hours before incubation with 0.5 μM FAM-ATTO647N aC′ dot nanosensors for 60 minutes. Confocal images were collected as described above with the addition of a 561/570 nm ex/em filter set for RFP collection.

Image processing and analyses. All image processing was performed in ImageJ, using built-in functions and publicly available plugins. STORM-assisted NN interpolation and quantitative cluster analysis were performed using custom Matlab codes.

Single particle pH calibration: Image areas where particles visibly colocalize in the FAM and ATTO647N channels were chosen, and each pixel was divided to obtain a FAMIATTO647N ratio before ratiometric pixels were averaged over the area of a single particle. Each pH point was represented by the numerical average and standard deviation of the ratiometric-pixel-averages of 5 or more particles.

Increasing number of particles averaged over to find ensemble pH calibration from single particle images: Square image areas were selected to include either 10 or 25 visible particles in the ATTO647N channel, as well as corresponding areas in the FAM channel. All pixels, in either the ATTO647N or FAM channel, below background level were removed via thresholding and set to NaN (Not a Number), and pixels with valid values were averaged across the selected areas (NaN pixels sum up to zero and are not accounted for in image area). The mean pixel-values from FAM and ATTO647N channels were then divided to obtain a ratio. Each pH point was represented by the numerical average and standard deviation of the ratios obtained from 10 or more sets of image areas in both channels.

Calculation of average whole-cell pH values as a function of incubation time: The pH at each pixel was determined by taking the ratio of the sensor to reference channels and applying the microscope-derived calibration curve (see FIGS. 5A-5F). Background values were removed via subtraction from pixel values in both sensor and reference channels, therefore, all pH pixels resulting from reference values lower than the background threshold were set to NaN (Not a Number). 472 px×472 px (50 μm×50 μm) single cell confocal images with no fewer than 5 visibly isolated ROIs in each cell were chosen for this analysis. Whole-cell average pH values were determined by the mean value of all pH pixels within the image (NaN pixels sum up to zero and are not accounted for in image area). 3 or more cell images were analyzed for each time point, and their mean pixel values were utilized to obtain averages and standard deviations.

STORM: STORM analysis was performed using the ThunderSTORM plugin developed by Ovesny et al. As described earlier, for optimal localization, maximum likelihood-fitting with a two-dimensional Gaussian point spread function (PSF) was employed. Localizations with lateral uncertainty >5 nm were removed before further analysis.

STORM-assisted NN interpolation: Images with binary pixels (scatter plot mode in ThunderSTORM visualization) mapping the central location of each PSF were multiplied by their corresponding TTRF-based pH images, resulting in “pH scatter plots”. Each pixel, now representing the location and pH value of each PSF, was replaced by a numerical average of the pixel values of its 24 nearest PSF neighbors (all pixels without PSFs were set to 0 and not considered). The distances between PSF locations were calculated by a simple 2D distance equation: D=\sqrt{(Xᵢ−Xⱼ)²+(Yᵢ−Yⱼ)²}. The interpolated pH scatter plot can then be used for quantitative cluster analysis, or a 9×9 pixel binarized-PSF can be applied to each scatter point for visual representation.

Quantitative cluster analysis: Clusters with more than 25 emitters within 500 nm distance of each other were identified and displayed. Number of emitters and average cluster diameter were obtained following previously published methods. Average cluster pH values were obtained from numeric averages of the pH pixel values within the same clusters.

Methods. Fluorescence Correlation Spectroscopy (FCS). Fluorescence correlation spectroscopy (FCS) was used to characterize nanosensor hydrodynamic diameter, concentration, and dyes per nanosensor. A dichroic mirror reflected and focused red solid state laser beam onto the object plane of a water immersion microscope objective (Zeiss Plan-Neofluar 63x NA 1.2). An avalanche photodiode detector (SPCM-AQR-14, PerkinElmer) collected the fluorescence through the same objective, after the fluorescence was spatially filtered by a 50-μm pinhole located at the image plane. A hardware correlator card (Flex03LQ, Correlator.com) correlated the time traces. For system alignment and focal volume size determination, we used Alexa Fluor 647 as a dye standard for the red laser line due to its known diffusion coefficient and photostability.

Three sets of five 30-second runs were recorded for each sample. Using a correlation function accounting for singlet-triplet transitions (equation (1)), the correlation data were then fitted.

$$G ( τ ) = 1 + \frac{1}{N} \cdot {( 1 + \frac{τ}{{τ}_{D}} )}^{- 1} \cdot {( 1 + \frac{τ}{{κ}^{2} {τ}_{D}} )}^{- \frac{1}{2}} \cdot \frac{1}{1 - F} \cdot ( 1 - F + F \cdot {e}^{- \frac{t}{{t}_{F}}} ) \qquad ( 1 )$$

Here, N is the mean number of particles within the detection volume, k is the structure factor determined by the ratio of the axial and radial radii (ω_{z} and ω_{xy}, respectively) of the observation volume, and τ_{D} is the characteristic diffusion time of an object through the observation volume. τ_{D} is defined as τ_{D}=ω_{xy}/4D, where D is the respective diffusion coefficient. F is the time- and space-averaged fraction of fluorophores that have entered the triplet state, and τ_{F} is the characteristic triplet state relaxation time. To determine nanosensor diameters, the Stokes-Einstein relation, equation (2), was then applied.

$$d = 2 \frac{{k}_{b} T}{6 π η D} \qquad ( 2 )$$

Here, k_{b} is the Boltzmann constant, T is the temperature, and η is the dynamic viscosity. The average number of dyes per particle, n, was calculated according to equation (3):

$$n = \frac{{c}_{d y e}}{{c}_{p a r t \dot{ι} c l e}} \qquad ( 3 )$$

Here, C_{dye} is the measured dye concentration derived from the dye extinction coefficient using the relative absorbance, and C_{particle} is the particle concentration determined by FCS.

Intra-endosomal Diffusion Length Calculation. Particle displacement by 2D diffusion within an endosome is estimated by the Stokes Einstein relation (4), and the 2D diffusion length equation (5).

$$D = \frac{{k}_{b} T}{6 π η r} \qquad ( 4 )$$

$$x = \sqrt{4 Dt} \qquad ( 5 )$$

Here, D is the diffusion coefficient, k_{b} is the Boltzmann constant, T is the temperature, η is the dynamic viscosity, r is the particle radius, x is the 2D diffusion length (displacement), and t is the diffusion time.

Based on the FCS experiments described above, D for a 4.8 nm aC′ dot diffusing in 20° C. water is calculated to be 89.3 μm²/s. Using an endosomal viscosity previously reported (2.2-fold of water), the intra-endosomal D for a 4.8 nm aC′ dot is 40.6 μm²/s. Using the 50 ms integration time of a single frame as the maximum diffusion time, we obtain a 2D diffusion length of 2.85 μm.

Although the present disclosure has been described with respect to one or more particular embodiments and/or examples, it will be understood that other embodiments and/or examples of the present disclosure may be made without departing from the scope of the present disclosure.

1. A method of determining a presence or an absence of an analyte or a concentration of an analyte in a sample or a portion thereof or an individual or a portion thereof using one or more aluminosilicate nanoparticle(s) comprising:
contacting the sample or individual with a plurality of aluminosilicate nanoparticles, each aluminosilicate nanoparticle comprising:
one or more reference dye groups(s), where each reference dye group is covalently bound to and encapsulated in the network of the aluminosilicate nanoparticle,
one or more sensing dye groups(s), wherein the sensing groups are capable of interacting with the sample or the portion thereof or the individual or the portion thereof, and
a plurality of polyethylene glycol (PEG) groups disposed on at least a portion of an surface or all of the surfaces of the aluminosilicate nanoparticle;
optionally, incubating the aluminosilicate nanoparticles with the sample or in the individual;
determining a presence or an absence or a concentration of the analyte in an individual region of a first object plane using ratiometric sensing;
localizing with resolution below Abbe's diffraction limit at least a portion of or all of the individual aluminosilicate nanoparticles in a second object plane, wherein the second object plane corresponds to at least a portion or all of the first object plane, using optical super-resolution microscopy (OSRM) imaging;
determining a presence or an absence of the analyte or the concentration of the analyte substantially at or at the position of one or more of the aluminosilicate nanoparticles using the presence or the absence or the concentration of the analyte obtained using the ratiometric sensing and the localization of the aluminosilicate nanoparticles obtained using OSRM; and
averaging the fluorescence intensity ratio of a desired number of individual aluminosilicate nanoparticles in proximity to an individual aluminosilicate nanoparticle to assign an average fluorescence intensity ratio to the individual aluminosilicate nanoparticle, wherein the average fluorescence intensity ratio assigned to the individual aluminosilicate nanoparticle corresponds to the presence or the absence of the analyte or the concentration of the analyte in the sample or the portion thereof or the individual or the portion thereof.

2. The method of claim 1, wherein the determining the presence or the absence or the local concentration of the analyte in the individual region of a detecting plane using ratiometric sensing and the localizing with resolution below Abbe's diffraction limit at least a portion of or all of the individual aluminosilicate nanoparticles in the second object plane are each carried out using OSRM imaging.

3. The method of claim 1, wherein the presence or the absence of the analyte or the concentration of the analyte in the sample or the portion thereof or the individual or the portion thereof is determined substantially at one or more of the aluminosilicate nanoparticle(s).

4. The method of claim 1, wherein the method comprises an OSRM method chosen from ground state depletion (GSD) microscopy, stochastic optical reconstruction microscopy (STORM), direct stochastic optical reconstruction microscopy (dSTORM), stimulated emission and depletion (STED), and photoactivated localization microscopy (PALM).

5. The method of claim 1, wherein the aluminosilicate nanoparticles are chosen from:
aluminosilicate core-organic ligand shell nanoparticles, each of the aluminosilicate core-organic ligand shell nanoparticles comprising:
an aluminosilicate core,
one or more reference dye group(s) covalently bound to and encapsulated in the aluminosilicate network of the aluminosilicate core-organic ligand shell nanoparticle,
one or more sensing dye group(s) capable of analyte sensing covalently bound to the aluminosilicate core network, wherein the one or more reference dye group(s) and the one or more sensing dye group(s) do not interfere with each other and/or one or more sensing dye group(s) capable of analyte sensing is/are disposed on at least a portion of or all of a surface or at least a portion of or all of the surfaces of the aluminosilicate core, and
a plurality of PEG groups disposed on at least a portion of a surface or all of the surfaces of the aluminosilicate core;
aluminosilicate core-aluminosilicate shell-organic shell nanoparticles, each of the aluminosilicate core-aluminosilicate shell-organic shell nanoparticles comprising:
an aluminosilicate core,
one or more reference dye group(s) covalently bound to and encapsulated in the aluminosilicate network of the aluminosilicate core,
an aluminosilicate shell disposed on at least a portion of or all of a surface or at least a portion of or all of the surfaces of the aluminosilicate core,
one or more sensing dye group(s) capable of analyte sensing covalently bound to and encapsulated in the aluminosilicate network of the aluminosilicate shell,
optionally, one or more sensing dye group(s) capable of analyte sensing disposed on at least a portion of or all of a surface or a portion of or all of the surfaces of the aluminosilicate shell, and
a plurality of PEG groups disposed on at least a portion of a surface or all of the surfaces of the aluminosilicate shell; and
any combination thereof.

6. The method of claim 1, wherein the aluminosilicate nanoparticles individually have at least one dimension of about 2 nm to about 10 nm.

7. The method of claim 1, wherein the aluminosilicate nanoparticles individually further comprise one or more targeting group(s), one or more therapeutic group(s), one or more diagnostic group(s), or any combination thereof.

8. The method of claim 1, wherein the analyte is chosen from hydrogen ions, oxidants, antioxidants, oxygen, reactive oxygen species (ROS), nitric oxide, chloride ions, metals, and metal ions.

9. The method of claim 1, wherein the analyte is hydrogen ions and the local pH substantially at or at the position of at least a portion or all of the aluminosilicate nanoparticles in the sample or the portion thereof or the individual or the portion thereof is determined.

10. The method of claim 1, wherein the individual sensing dye group(s) is/are capable of sensing pH, sensing redox status, sensing the presence or absence of oxygen, sensing the presence or absence of reactive oxygen species (ROS), sensing the presence or absence of chloride ions, sensing the presence or absence of nitric oxide, or sensing the presence or absence of one or more metal(s) and/or metal ion(s).

11. The method of claim 1, wherein the contacting is administering the composition to the individual.

12. A method of targeting, diagnosing, treating, preventing, or any combination thereof, a current or potential disease, disease state, condition, disorder, side effect, or any combination thereof, in an individual, the method comprising a method of claim 1.

13. The method of claim 12, wherein the sample is a biopsy sample or a resected tissue sample.

14. The method of claim 12, wherein the current or potential disease, disease state, condition, disorder, side effect, or any combination thereof, is chosen from infections, cancers, neurological conditions/diseases, neurodegenerative diseases, psychological conditions/diseases, inflammatory conditions/diseases, cardio-vascular diseases, and any combination thereof.

15. The method of claim 14, wherein the current or potential disease is cancer, and the method further comprises one or more chemotherapy treatment(s), one or more radiation treatment(s), one or more photodynamic therapy treatment(s), one or more surgical intervention(s), or the like, or any combination thereof.

16. The method of claim 14, wherein the method further comprises visualization of abnormal cells after administration of the aluminosilicate nanoparticles.

17. The method of claim 16, wherein the visualization is carried out using fluorescence imaging.

18. A kit comprising one or more aluminosilicate nanoparticle(s) and/or a composition comprising the aluminosilicate nanoparticle(s), and instructions for use of the aluminosilicate nanoparticles and/or the composition(s) for carrying out a method of claim 1.

19. The kit of claim 18, wherein the aluminosilicate nanoparticles are chosen from:
aluminosilicate core-organic ligand shell nanoparticles, each of the aluminosilicate core-organic ligand shell nanoparticles comprising:
an aluminosilicate core,
one or more reference dye group(s) covalently bound to and encapsulated in the aluminosilicate network of the aluminosilicate core-organic ligand shell nanoparticle,
one or more sensing dye group(s) capable of analyte sensing covalently bound to the aluminosilicate core network, wherein the one or more reference dye group(s) and the one or more sensing dye group(s) do not interfere with each other and/or one or more sensing dye group(s) capable of analyte sensing is/are disposed on at least a portion of or all of a surface or at least a portion of or all of the surfaces of the aluminosilicate core, and
a plurality of PEG groups disposed on at least a portion of a surface or all of the surfaces of the aluminosilicate core;
aluminosilicate core-aluminosilicate shell-organic shell nanoparticles, each of the aluminosilicate core-aluminosilicate shell-organic shell nanoparticles comprising:
an aluminosilicate core,
one or more reference dye group(s) covalently bound to and encapsulated in the aluminosilicate network of the aluminosilicate core,
an aluminosilicate shell disposed on at least a portion of or all of a surface or at least a portion of or all of the surfaces of the aluminosilicate core,
one or more sensing dye group(s) capable of analyte sensing covalently bound to and encapsulated in the aluminosilicate network of the aluminosilicate shell,
optionally, one or more sensing dye group(s) capable of analyte sensing disposed on at least a portion of or all of a surface or a portion of or all of the surfaces of the aluminosilicate shell, and
a plurality of PEG groups disposed on at least a portion of a surface or all of the surfaces of the aluminosilicate shell; and
any combination thereof.

20. The kit of claim 18, wherein the aluminosilicate nanoparticles individually have at least one dimension of about 2 to about 10 nm.
